(19) United States
(12) Patent Application Publication
(10) Pub. No.: US 2022/0235353 A1
(43) Pub. Date: Jul. 28, 2022

(54) PROTECTIVE ELEMENTS FOR NUCLEIC ACID SYNTHETIC BIOLOGY

(71) Applicant: California Institute of Technology, Pasadena, CA (US)

(72) Inventors: Lisa Hochrein, Pasadena, CA (US); Heyun Li, Pasadena, CA (US); Evan Mun, Pasadena, CA (US); Paul W. Rothemund, Pasadena, CA (US); Niles A. Pierce, Pasadena, CA (US)

(21) Appl. No.: 17/584,271

(22) Filed: Jan. 25, 2022

Related U.S. Application Data
(60) Provisional application No. 63/141,865, filed on Jan. 26, 2021.
(60) Provisional application No. 63/181,802, filed on Apr. 29, 2021.

(51) Int. Cl. C12N 15/11; C12N 9/22; C12N 15/113; C12N 15/10

(52) U.S. Cl. CPC C12N 15/111; C12N 9/22; C12N 2310/20; C12N 15/102; C12N 15/113

(57) ABSTRACT

Nucleic acids (DNA and RNA) provide a versatile platform for engineering synthetic biology in a variety of technology areas including medicine, science, agriculture, and energy. In many settings, degradation of nucleic acid molecules poses a significant engineering challenge as the molecules do not function if they have been degraded. In some embodiments, nucleic acid protective elements (PELs) are used to protect chemically synthesized or expressed nucleic acid molecules from degradation. PELs may be derived from all or part of a viral xrRNA sequence and/or structural motif, PELs may include rationally designed sequences and/or structural motifs, PELs may be engineered using directed evolution, and in some embodiments, PELs comprise a mixture of biologically derived, rationally designed sequence and/or structural motifs, and/or sequences and/or structural motifs that are engineered by directed evolution. In some embodiments, PELs significantly enhance the performance of nucleic acid synthetic biology, protecting nucleic acid regulatory and/or structural elements from degradation to increase regulatory dynamic range, fractional dynamic range, fold-change, and/or other performance metrics. In some embodiments, PELs that reduce nucleic acid degradation provide a platform technology for enhancing the performance of synthetic biology, with applications including therapeutics, diagnostics, biological research tools, vaccines, crop protection, molecular manufacturing, sustainable energy production, and other areas involving nucleic acids.

### STATEMENT REGARDING FEDERALLY SPONSORED R&D

This invention was made with government support under Grant No. HR0011-17-2-0008 awarded by DARPA, under Grant No. NNX16AO69A and Grant No. 7000000323 awarded by NASA, and with support from a National Science Foundation Graduate Research Fellowship under Grant No. DGE-1745301. The government has certain rights in the invention.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### REFERENCE TO SEQUENCE LISTING, TABLE, OR COMPUTER PROGRAM LISTING

The present application is being filed along with a Sequence Listing in electronic format. The Sequence Listing is provided as a file entitled CALTE156ASEQLIST.txt created on Jan. 21, 2022 and is 64,857 bytes in size. The information in the electronic format of the Sequence Listing is incorporated herein by reference in its entirety.

### BACKGROUND

Nucleic acids (DNA and RNA) provide a versatile platform for engineering synthetic biology in a variety of arenas including medicine, science, agriculture, and energy, with applications including therapeutics, diagnostics, biological research tools, vaccines, crop protection, molecular manufacturing, and sustainable energy production. In some settings, the sequence of a nucleic acid molecule is translated into a protein that implements a function. For example, different messenger RNAs (mRNAs) can be translated into enzymes, membrane proteins, motor proteins, etc. In other settings, the nucleic acid molecule directly implements a function without being translated into a protein. For example, guide RNAs (gRNAs), microRNAs (miRNAs), transfer RNAs (tRNAs), and other non-coding RNAs (ncRNAs) all carry out different functions by directly exploiting the affinity and selectivity of nucleic acid base-pairing. gRNAs mediate induction, silencing, editing, binding, epigenome editing, chromatin interaction mapping and regulation, or imaging of a complementary target gene by the CRISPR/Cas pathway. microRNAs mediate post-transcriptional regulation of partially complementary target genes by the RNA interference (RNAi) pathway. As an mRNA is being translated by the ribosome, tRNAs bind to complementary codons within the mRNA to supply the amino acids that are added to the growing polypeptide chain. DNA and RNA molecules can also be engineered to assemble into diverse functional structures, devices, and systems. Nucleic acid molecules can be designed to interact and change conformation via prescribed self-assembly and disassembly pathways so as to implement or mediate diverse functions including signal transduction, catalysis, logic, and regulation. Functional nucleic acid molecules can be engineered for use in diverse settings from cell-free systems, to cultured cells, environmental samples, developing embryos, humans, pets, livestock, crops, gut microbiomes, wounds, ecosystems, and the biosphere.

### SUMMARY OF THE INVENTION

In many settings, degradation of nucleic acid molecules by nucleases poses a significant engineering challenge as the molecules do not function if they have been degraded. RNA degradation can also occur via non-enzymatic auto-hydrolysis in which the 2′ hydroxyl of the ribose interacts with the adjacent phosphorus to break the phosphodiester bond in the RNA backbone. One traditional approach to combatting nucleic acid degradation is to synthesize chemically modified nucleic acids or nucleic acid analogs (for example, LNA, PNA, XNA, 2′OMe-RNA and phosphorothioate backbone modifications, or combinations thereof) that inhibit nuclease recognition and/or auto-hydrolysis to impede degradation. This approach has been pursued extensively in development of chemotherapies that down-regulate a gene of choice using chemically modified antisense nucleic acids (asRNA or asDNA) or small interfering RNAs (siRNAs) that are delivered into the patient. However, each delivery event introduces a finite supply of the regulatory molecule that must then be replenished by a new delivery event in order to maintain a supply in the cell. In synthetic biology contexts, another approach to counteracting nucleic acid degradation is to increase the expression level of RNAs that are being degraded so as to ensure that sufficient quantities survive to perform the intended function. By relying on unmodified RNA expressed within the cell, the supply of the degraded RNAs can be replenished continuously. However, increasing expression levels of exogenous nucleic acids places a heavy metabolic load on the cell that often leads to toxicity—a major drawback that undermines performance. In nature, viruses use a different approach to protect against degradation by incorporating exoribonuclease-resistant RNA (xrRNA) motifs that form a mechanical block to halt diverse exoribonucleases.¹⁻⁹

In some embodiments, nucleic acid protective elements (PELs) are used to protect chemically synthesized or expressed nucleic acid molecules from degradation. In some embodiments, PELs are derived from all or part of a viral xrRNA structural motif and/or sequence. In some embodiments, a PEL comprises a structured region that reduces non-enzymatic degradation of a protected nucleic acid 5′ and/or 3′ of the PEL. In some embodiments, PEL structural motifs and/or sequences are rationally designed. In some embodiments, PEL structural motifs and/or sequences are engineered by directed evolution. In some embodiments, PELs comprise a mixture of biologically derived, rationally designed, and/or directed-evolution engineered structural motifs and/or sequences. In some embodiments, PELs significantly enhance the performance of nucleic acid synthetic biology, protecting nucleic acid regulatory and/or structural elements from degradation to increase regulatory dynamic range, fractional dynamic range, fold-change, and/or other performance metrics. In some embodiments, PELs that form a mechanical block against nuclease degradation provide a platform technology for enhancing the performance of nucleic acid synthetic biology. In some embodiments, PEL-mediated improvements in the performance of synthetic biology impact applications in medicine, science, agriculture, and/or energy, including therapeutics, diagnostics, biological research tools, vaccines, crop protection, molecular manufacturing, and/or sustainable energy production.

In accordance with some implementations, there is a protective element (PEL) within a synthesized or expressed RNA molecule that reduces degradation of a sequence element 5′ and/or 3′ of the PEL, wherein the sequence element that experiences reduced degradation is known as a protected sequence.

In accordance with some implementations, there is a protective element (PEL) within a nucleic acid, wherein the PEL comprises a structured region comprising one or more duplexes, and wherein the structured region reduces degradation of a protected sequence 5′ and/or 3′ of the PEL.

In accordance with some implementations, there is a method of reducing degradation of a nucleic acid in a sample, comprising: providing a synthesized or expressed RNA molecule that includes a protective element (PEL); and combining the RNA molecule including the PEL with a sample comprising at least one other molecule; wherein the PEL reduces degradation of a sequence element 5′ and/or 3′ of the PEL and the sequence element that experiences reduced degradation is known as a protected sequence.

In accordance with some implementations, there is a method of reducing degradation of a nucleic acid in a sample, comprising: providing a protective element (PEL) within a nucleic acid; and combining the nucleic acid containing the PEL with a sample comprising at least one other molecule; wherein the PEL comprises a structured region that reduces degradation of a protected sequence 5′ and/or 3′ of the PEL.

In some implementations, the PEL comprises a PEL motif comprising a pseudoknot motif: the pseudoknot motif comprising (from 5′ to 3′) a 1^{st} segment, a 2^{nd} segment, a 3^{rd} segment, a 4^{th} segment, a 5^{th} segment, a 6^{th} segment, a 7^{th} segment, and an 8^{th} segment, wherein the 1^{st} segment hybridizes to the 7^{th} segment to form a 1^{st} duplex, the 2^{nd} segment hybridizes to the 3^{rd} segment to form a 2^{nd} duplex, the 4^{th} segment hybridizes to the 6^{th} segment to form a 3^{rd} duplex, and the 5^{th} segment hybridizes to the 8^{th} segment to form a 4^{th} duplex.

In some implementations, the PEL comprises a PEL motif comprising (from 5′ to 3′) a pseudoknot motif and a hairpin motif: the pseudoknot motif comprising (from 5′ to 3′) a 1^{st} segment, a 2^{nd} segment, a 3^{rd} segment, a 4^{th} segment, a 5^{th} segment, a 6^{th} segment, a 7^{th} segment, and an 8^{th} segment, wherein the 1^{st} segment hybridizes to the 7^{th} segment to form a 1^{st} duplex, the 2^{nd} segment hybridizes to the 3^{rd} segment to form a 2^{th} duplex, the 4^{th} segment hybridizes to the 6^{th} segment to form a 3^{rd} duplex, the 5^{th} segment hybridizes to the 8^{th} segment to form a 4^{th} duplex; and the hairpin motif comprising (from 5′ to 3′) a 9^{th} segment and a 10^{th} segment, wherein the 90^{th} segment hybridizes to the 10^{th} segment to form a 5^{th} duplex.

In some implementations, the PEL comprises a PEL motif comprising (from 5′ to 3′) a first pseudoknot motif and a second pseudoknot motif: the first pseudoknot motif comprising (from 5′ to 3′) a 1^{st} segment, a 2^{rd} segment, a 3^{th} segment, a 4^{th} segment, a 5^{th} segment, a 6^{th} segment, a 7^{th} segment, and an 8^{th} segment, wherein the 1^{st} segment hybridizes to the 7^{th} segment to form a 1^{st} duplex, the 2^{nd} segment hybridizes to the 3^{rd} segment to form a 2^{nd} duplex, the 4^{th} segment hybridizes to the 6^{th} segment to form a 3^{rd} duplex, and the 5^{th} segment hybridizes to the 8^{th} segment to form a 4^{th} duplex; and the second pseudoknot motif comprising (from 5′ to 3′) a 9^{th} segment, a 10^{th} segment, an 11^{th} segment, a 12^{th} segment, a 13^{th} segment, a 14^{th} segment, a 15^{th} segment, and a 16^{th} segment, wherein the 9^{th} segment hybridizes to the 15^{th} segment to form a 5^{th} duplex, the 10^{th} segment hybridizes to the 11^{th} segment to form a 6^{th} duplex, the 12^{th} segment hybridizes to the 14^{th} segment to form a 7^{th} duplex, and the 13^{th} segment hybridizes to the 16^{th} segment to form an 8^{th} duplex.

In some implementations, the PEL comprises a PEL motif comprising (from 5′ to 3′) a first pseudoknot motif, a first hairpin motif, a second pseudoknot motif, and a second hairpin motif: the first pseudoknot motif comprising (from 5′ to 3′) a 1^{st} segment, a 2^{nd} segment, a 3^{rd} segment, a 4^{th} segment, a 5^{th} segment, a 6^{th} segment, a 7^{th} segment, and an 8^{th} segment, wherein the 1^{st} segment hybridizes to the 7^{th} segment to form a 1^{st} duplex, the 2^{nd} segment hybridizes to the 3^{rd} segment to form a 2^{nd} duplex, the 4^{th} segment hybridizes to the 6^{th} segment to form a 3^{rd} duplex, and the 5^{th} segment hybridizes to the 8^{th} segment to form a 4^{th} duplex; the first hairpin motif comprising (from 5′ to 3′) a 9^{th} segment and a 10^{th} segment, wherein the 9^{th} segment hybridizes to the 10^{th} segment to form a 5^{th} duplex; the second pseudoknot motif comprising (from 5′ to 3′) an 11^{th} segment, a 12^{th} segment, a 13^{th} segment, a 14^{th} segment, a 15^{th} segment, a 16^{th} segment, a 17^{th} segment, and an 18^{th} segment, wherein the 11^{th} segment hybridizes to the 17^{th} segment to form a 6^{th} duplex, the 12^{th} segment hybridizes to the 13^{th} segment to form a 7^{th} duplex, the 14^{th} segment hybridizes to the 16^{th} segment to form an 8^{th} duplex, and the 15^{th} segment hybridizes to the 18^{th} segment to form a 9^{th} duplex; and the second hairpin motif comprising (from 5′ to 3′) a 19^{th} segment and a 20^{th} segment, wherein the 19^{th} segment hybridizes to the 20^{th} segment to form a 10^{th} duplex.

In some implementations, the PEL comprises a PEL motif comprising a pseudoknot motif: the pseudoknot motif comprising (from 5′ to 3′) a 1^{st} segment, a 2^{nd} segment, a 3^{rd} segment, a 4^{th} segment, a 5^{th} segment, a 6^{th} segment, a 7^{th} segment, an 8^{th} segment, a 9^{th} segment, and a 10^{th} segment, wherein the 1^{st} segment hybridizes to the 9^{th} segment to form a 1^{st} duplex, the 2^{nd} segment hybridizes to the 8^{th} segment to form a 2^{nd} duplex, the 3^{rd} segment hybridizes to the 4^{th} segment to form a 3^{rd} duplex, the 5^{th} segment hybridizes to the 7^{th} segment to form a 4^{th} duplex, and the 6^{th} segment hybridizes to the 10^{th} segment to form a 5^{th} duplex.

In some implementations, the PEL comprises a PEL motif comprising a pseudoknot motif: the pseudoknot motif comprising (from 5′ to 3′) a 1^{st} segment, a 2^{nd} segment, a 3^{rd} segment, a 4^{th} segment, a 5^{th} segment, and a 6^{th} segment, wherein the 1^{st} segment hybridizes to the 5^{th} segment to form a 1^{st} duplex, the 2^{nd} segment hybridizes to the 4^{th} segment to form a 2′ duplex, and the 3^{rd} segment hybridizes to the 6^{th} segment to form a 3^{rd} duplex.

In some implementations, the PEL comprises a PEL motif comprising a pseudoknot motif: the pseudoknot motif comprising (from 5′ to 3′) a 1^{st} segment, a 2^{nd} segment, a 3^{rd} segment, and a 4^{th} segment, wherein the 1^{st} segment hybridizes to the 3^{rd} segment to form a 1^{st} duplex and the 2^{nd} segment hybridizes to the 4^{th} segment to form a 2^{nd} duplex.

In some implementations, the PEL comprises a PEL motif comprising a pseudoknot motif: the pseudoknot motif comprising (from 5′ to 3′) a 1st segment, a 2nd segment, a 3^{rd} segment, and a 4^{th} segment, wherein the 1^{st} segment hybridizes to the 3^{rd} segment to form a structured region comprising a 1^{st} duplex and the 2^{nd} segment hybridizes to the 4^{th} segment to form a 2^{nd} duplex.

In some implementations, the PEL comprises a PEL motif comprising a pseudoknot motif: the pseudoknot motif comprising (from 5′ to 3′) a 1st segment, a 2nd segment, a 3^{rd} segment, a 4^{th} segment, a 5^{th} segment, and a 6^{th} segment, wherein the 1^{st} segment hybridizes to the 3^{rd} segment to form a 1^{st} structured region comprising a 1^{st} duplex, the 2^{nd} segment hybridizes to the 5^{th} segment to form a 2^{nd} duplex, and the 4^{th} segment hybridizes to the 6^{th} segment to form a 2^{nd} structured region comprising a 3^{rd} duplex.

In some implementations, the PEL comprises a PEL motif comprising a pseudoknot motif: the pseudoknot motif comprising (from 5′ to 3′) a 1^{st} segment, a 2^{nd} segment, a 3^{rd} segment, a 4^{th} segment, a 5^{th} segment, and a 6^{th} segment, wherein the 1^{st} segment hybridizes to the 3^{rd} segment to form a 1^{st} structured region comprising a 1^{st} duplex, the 2^{nd} segment hybridizes to the 5^{th} segment to form a 2^{nd} duplex, and a 3^{rd} duplex is formed within a 2^{nd} structured region by hybridization between two sub-segments of the 4^{th} segment or between two sub-segments of the 6^{th} segment.

In some implementations, the PEL comprises a PEL motif comprising a pseudoknot motif: the pseudoknot motif comprising (from 5′ to 3′) a 1^{st} segment, a 2^{nd} segment, a 3^{rd} segment, and a 4^{th} segment, wherein the 1st segment hybridizes to the 3^{rd} segment to form a 1^{st} duplex and the 2^{nd} segment hybridizes to the 4^{th} segment to form a structured region comprising a 2^{nd} duplex.

In some implementations, the PEL comprises a PEL motif comprising a structured region, the structured region comprising a first duplex, wherein the structured region serves as a mechanical block to inhibit nuclease degradation of the protected sequence.

In some implementations, additional base-pairing and/or tertiary contacts form within the PEL motif, including but not limited to base pairs, base triples, base-phosphate interactions, and base-base interactions.

In some implementations, consecutive motifs within a PEL (from 5′ to 3′) are connected by a linker comprising zero, one, or more nucleotides or alternatively comprising a material not capable of base-pairing.

In some implementations, the PEL reduces degradation of an exogenous RNA molecule in a eukaryotic cell.

In some implementations, the protected sequence is an mRNA vaccine or an RNA drug.

In some implementations, the protected sequence mediates the function of an endogenous biological pathway; functions as a regulator; functions as a logic gate that accepts one or more inputs and conditionally produces one or more outputs; serves as a structural element in an assembly of multiple structural elements; is translated by an in vitro translation system, and/or serves as a substrate for mediating the interaction of other molecules.

In some implementations, the protected sequence mediates the function of the CRISPR/Cas pathway.

In some implementations, the protected sequence is a trigger sequence that activates a previously inactive conditional guide RNA (cgRNA), allowing the cgRNA to direct Cas-mediated induction, silencing, editing, binding, epigenome editing, chromatin interaction mapping and regulation, or imaging of a target gene within a eukaryotic cell.

In some implementations, the protected sequence is a trigger sequence that inactivates a previously active conditional guide RNA, stopping the cgRNA from further directing Cas-mediated induction, silencing, or editing, binding, epigenome editing, chromatin interaction mapping and regulation, or imaging of a target gene within a eukaryotic cell.

In some implementations, the PEL comprises RNA, DNA, 2′OMe-RNA, chemically modified nucleic acids, synthetic nucleic acid analogs, PNA, XNA, any other material capable of base-pairing, one or more chemical linkers not capable of base-pairing, or any combination thereof.

In some implementations, the protected sequence comprises RNA, DNA, 2′ OMe-RNA, chemically modified nucleic acids, synthetic nucleic acid analogs, PNA, XNA, any other material capable of base-pairing, one or more chemical linkers not capable of base-pairing, or any combination thereof.

### BRIEF DESCRIPTION OF THE DRAWINGS

FIG. 1 depicts examples of design elements for nucleic acid synthetic biology.

FIG. 2 depicts examples of signal transduction using nucleic acid synthetic biology.

FIG. 3 depicts examples of contexts in which RNA degradation presents challenges to nucleic acid nanotechnology and nucleic acid synthetic biology.

FIGS. 4A-4L depict examples of protective elements (PEL) sequences and structures.

FIGS. 5A-5B depict the logic, function, structure, and interactions of a standard guide RNA (gRNA).

FIG. 6A-6B depicts the logic and function of a conditional guide RNA (cgRNA).

FIGS. 7A-7E demonstrate enhancing nucleic acid synthetic biology performance using PELs in human cells.

FIGS. 8A-8E demonstrate enhancing nucleic acid synthetic biology performance for multiple orthogonal regulators using PELs in human cells.

FIGS. 9A-9D demonstrate enhancing nucleic acid synthetic biology performance using different PEL variants in human cells.

FIGS. 10A-10F demonstrate using PELs to protect exogenous RNAs from degradation in human cells.

FIGS. 11A-11E demonstrate using PELs to protect RNAs from exoribonuclease digestion.

FIGS. 12A-12G demonstrate using a PEL to block exoribonuclease digestion of the portion of an RNA that is 3′ of the PEL.

FIGS. 13A-13F demonstrate using different PEL variants to protect RNA from exoribonuclease digestion.

FIGS. 14A-14B depict PEL motifs (Type 1) comprising a pseudoknot motif.

FIGS. 15A-15B depict PEL motifs (Type 2) comprising a pseudoknot motif and a hairpin motif.

FIGS. 16A-16B depict PEL motifs (Type 3) comprising a first pseudoknot motif and a second pseudoknot motif.

FIGS. 17A-17B depict PEL motifs (Type 4) comprising a first pseudoknot motif, a first hairpin motif, a second pseudoknot motif, and a second hairpin motif.

FIGS. 18A-18B depict PEL motifs (Type 5) comprising a pseudoknot motif.

FIGS. 19A-19B depict PEL motifs (Type 6) comprising a pseudoknot motif.

FIGS. 20A-20B depict example target test tubes for computational sequence design of PELs.

FIGS. 21A-21E depict examples of PEL structures.

FIGS. 22A-22B depict examples of PEL sequences.

FIGS. 23A-23B depict PEL motifs (Type 7) comprising a pseudoknot motif.

FIGS. 24A-24B depict PEL motifs (Type 8) comprising a pseudoknot motif comprising a structured region.

FIGS. 25A-25B depict PEL motifs (Type 9) comprising a pseudoknot motif two structured regions.

FIGS. 26A-26B depict PEL motifs (Type 10) comprising a pseudoknot motif comprising a structured region.

FIG. 27 depicts PEL motifs (Type 11) comprising a motif comprising a structured region.

FIGS. 28A-28F demonstrate enhancing nucleic acid synthetic biology performance using different PEL variants in human cells.

FIGS. 29A-29F demonstrate using different PEL variants to protect RNA from exoribonuclease digestion.

### DETAILED DESCRIPTION OF THE PREFERRED EMBODIMENT

The disclosure is generally related to nucleic acid protective elements that function to protect nucleic acids from degradation.

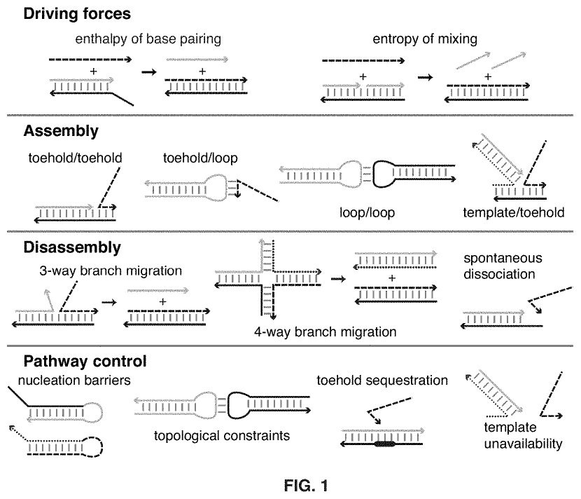

Dynamic nucleic acid nanotechnology enables engineering of complex pathway-controlled hybridization cascades in which nucleic acid strands (for example, small conditional DNAs (scDNAs) or small conditional RNAs (scRNAs)) execute dynamic functions by autonomously performing interactions and conformation changes in a prescribed order.^{10,11} Pathway-controlled self-assembly and disassembly can be powered by the enthalpy of base-pairing¹²⁻²⁰ and/or the entropy of mixing^{16,17,19,21} ([0033] FIG. 1). Modes of nucleating interactions include toehold/toehold,^{12-17,21,22} loop/toehold,^{18,19} loop/loop,^{20,23} and template/toehold¹⁹ hybridization. Modes of strand displacement include 3-way branch migration,^{12-14,21,16,22,17,18} 4-way branch migration,^{15,19,23,24} and spontaneous dissociation.^{17,19,21} To exert control over the order of self-assembly and disassembly events, scDNAs and scRNAs can be designed to co-exist metastably (i.e., the molecules are kinetically trapped) or stably (i.e., the molecules are thermodynamically trapped), with the next step in the reaction pathway triggered either by a cognate molecular input detected from the environment or by a molecular output of a previous step in the reaction pathway. Principles for engineering conditional metastability include nucleation barriers,^{13,16} topological constraints,^{20,23} toehold sequestration,^{14,16,17,19,21} and template unavailability,¹⁹ while principles for engineering conditional stability include cooperativity¹¹ and sequence transduction.¹⁹ These design elements enable the rational design and construction of scDNAs and/or scRNAs executing diverse dynamic functions, including catalysis, signal amplification, sequence transduction, shape transduction, signal transduction, Boolean logic, and locomotion.^{10,11}

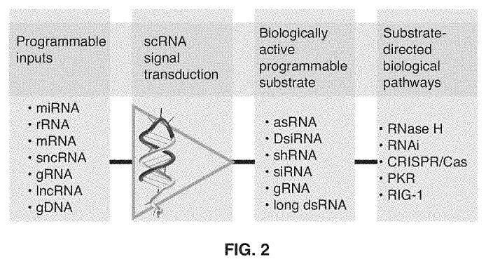

Dynamic nucleic acid nanotechnology makes it possible to introduce synthetic regulatory links within the chemically complex environment of living cells and organisms. For example, consider scRNAs that interact and change conformation to transduce between detection of an endogenous programmable input, and production of a biologically active programmable output recognized by an endogenous or exogenous biological pathway (FIG. 2). In this scenario, the input controls the scope of regulation and the output controls the target of regulation, with the scRNA performing signal transduction to create a logical link between the two.^{19,25-29} Any pathway that recognizes RNA (or DNA) is a potential candidate for conditional regulation by scRNAs (or scDNAs). Example inputs for scRNA signal transduction include miRNA, ribosomal RNA (rRNA), mRNA, small non-coding RNAs (sncRNA), gRNA, long non-coding RNA (lncRNA), and genomic DNA (gDNA). Example outputs of scRNA signal transduction include anti-sense RNA (asRNA), Dicer-substrate short interfering RNA (DsiRNA), short hairpin RNA (shRNA), small interfering RNA (siRNA), gRNA and long double strand RNA (dsRNA). Example biological pathways that can recognize the programmable outputs of scRNA signal transduction and perform scRNA-mediated conditional function include RNase H, RNAi, CRISPR/Cas, protein kinase R (PKR), or retinoic acid-inducible gene 1 (RIG-1). scRNAs enable restriction of synthetic regulation to a desired cell type, tissue, or organ without engineering the organism. For example, as a biological research tool, conditional gene silencing enables studies of genetic necessity and conditional gene activation enables studies of genetic sufficiency. This can be achieved by selecting an endogenous RNA trigger X with the desired spatial and temporal expression profile. To shift conditional regulation to a different tissue or developmental stage, an scRNA motif can be reprogrammed to recognize a different input X with the desired spatial and temporal expression profile. Multi-input logic (for example, “X1 AND X2” or “X1 OR X2”) can be used to further refine the scope of regulation, either by restricting the scope using “AND” or by increasing the scope using “OR”, or by further refining the scope using combinations of “AND” and “OR”. In a therapeutic context (with the input as a programmable disease marker and output as an independent programmable therapeutic pathway), scRNAs provide a basis for selective treatment of diseased cells leaving healthy cells untouched.

DNA can be programmed to self-assemble into diverse structural motifs and materials' as well as execute dynamic reaction pathways.³¹ RNA synthetic biology³²⁻³⁴ makes possible the regulation of gene expression and cellular behavior through diverse RNA-mediated mechanisms including aptamer-mediated riboswitches³², RNA transcriptional activators,^{35,36} toehold switches for conditional transcription,³⁷ small interfering RNAs (siRNAs) for RNA interference (RNAi), small conditional RNAs for cell-selective RNAi,^{19,26} guide RNAs and catalytically active Cas protein or catalytically dead Cas protein (dCas) for gene silencing, induction, editing, binding, epigenome editing, chromatin interaction mapping and regulation, or imaging,³⁸⁻⁴⁴ and conditional guide RNAs for cell-selective control of CRISPR/Cas.^{25,27-29} RNA synthetic biology can also be used to express structures and materials including RNA origami^{45,46}, structures that serve as substrates to template chemical reactions,⁴⁷ and structures that serve as templates for protein folding.⁴⁸ RNA synthetic biology has applications to diagnostics (for example detection of Ebola virus⁴⁹ and Zika virus⁵⁰), mRNA vaccines (including COVID-19 vaccines),⁵¹ mRNA drugs,⁵² CRISPR/Cas drugs,^{53,54} RNAi and antisense drugs.^{55,56}

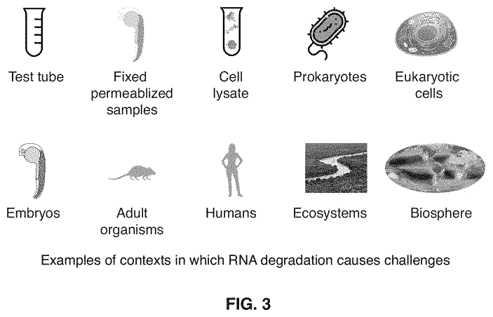

With nucleic acid synthetic biology, degradation of the nucleic acid components by nucleases remains a major challenge across diverse settings including test tubes on the bench top, fixed permeablized samples, cell lysates, prokaryotes, eukaryotic cells, embryos, adult organisms, humans, ecosystems, and the biosphere (FIG. 3). One approach to reducing degradation of nucleic acids in living cells is to use chemical modifications or synthetic nucleic acid analogs that reduce recognition by nucleases, including 2′OMe-RNA nucleotides, phosphorothioate backbone modifications, locked nucleic acid (LNA) nucleotides, peptide nucleic acid (PNA) nucleotides, xeno nucleic acid (XNA) nucleotides, and combinations thereof.⁵⁷⁻⁶³ With this strategy, chemically modified molecules must be delivered to the cell or organism since they cannot be synthesized by the endogenous machinery within the cell. Another approach is to over-express synthetic nucleic acids with the goal of saturating degradation pathways and ensuring that enough synthetic molecules remain to perform the desired function. This approach is metabolically inefficient, placing a heavy metabolic load on the cell that can cause toxicity and undermine utility.⁶⁴⁻⁶⁶

In some embodiments, any of the PELs provided herein can be all or part of an exoribonuclease-resistant RNA (xrRNA), a rationally designed RNA, an RNA engineered by directed evolution, or an RNA obtained from any combination of the above.

### Definitions

“Nucleic acids” as used herein includes oligomers of RNA, DNA, 2′ OMe-RNA, LNA, PNA, XNA, chemically modifications thereof, synthetic analogs of RNA or DNA, any other material capable of base-pairing, one or more chemical linkers not capable of base-pairing, or any combination thereof. Nucleic acids may include analogs of DNA or RNA having modifications to either the bases or the backbone. For example, nucleic acid, as used herein, includes the use of peptide nucleic acids (PNA). The term “nucleic acids” also includes chimeric molecules. The phrase includes artificial constructs as well as derivatives etc. The phrase includes, for example, any one or more of DNA, RNA, 2′OMe-RNA, LNA, XNA, synthetic nucleic acid analogs, and PNA. The phrase also includes oligomers of RNA, DNA, 2′OMe-RNA, LNA, PNA, XNA and/or other nucleic acid analogs with or without chemical linkers between nucleic acid segments.

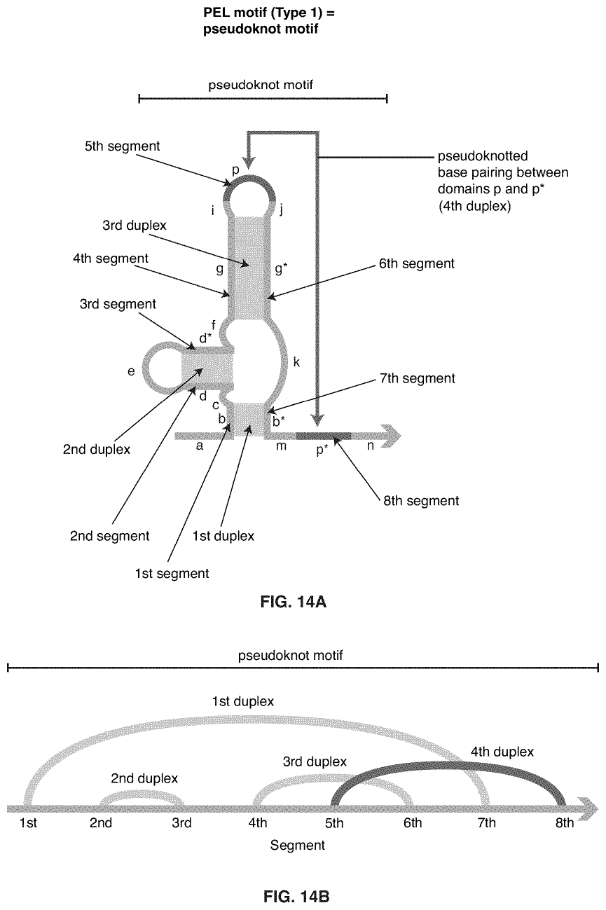

A “nucleic acid strand” refers to an oligomer of nucleotides (typically listed from 5′ to 3′) with or without the any of the variations defined for nucleic acids. In diagrams, a nucleic acid strand is depicted with an arrowhead at the 3′ end. A nucleic acid strand may comprise one or more “segments”, each comprising one or more consecutive nucleotides (or optionally zero nucleotides if a segment is optional). For example, FIG. 14A depicts a nucleic acid strand containing a 1^{st} segment, a 2^{nd} segment, a 3^{rd} segment, a 4^{th} segment, a 5^{th} segment, a 6^{th} segment, a 7^{th} segment, and an 8^{th} segment each comprising one or more “sequence domains”. A nucleic acid strand may comprise one or more “sequence domains” (or equivalently “domains”), each comprising one or more consecutive nucleotides (or optionally zero nucleotides if a domain is optional). For example, FIG. 14A depicts a nucleic acid strand comprising sequence domains “a”, “b”, “c”, “d”, “e”, “d*”, “f”, “g”, “i”, “p”, “j”, “g*”, “k”, “b*”, “m”, “p*”, “n”. In FIG. 14A, the 1^{st} segment corresponds to sequence domain “b”, the 2^{nd} segment corresponds to sequence domain “d”, the 3^{rd} segment corresponds to sequence domain “d*”, the 4^{th} segment corresponds to sequence domain “g”, the 5^{th} segment corresponds to sequence domain “p”, the 6^{th} segment corresponds to sequence domain “g*”, the 7^{th} segment corresponds to sequence domain “b*”, and the 8^{th} segment corresponds to sequence domain “p*”.

A “secondary structure” of a nucleic acid strand is defined by a set of base pairs (for example, Watson-Crick base pairs [A-U or C-G] or wobble base pairs [G-U] for RNA).

Two “complementary” segments (or sequence domains) can base-pair to each other (i.e., hybridize) to form a “duplex”, representing one or more consecutive base pairs between two segments (or equivalently, one or more consecutive base pairs between two sequence domains). For example, in FIG. 14A, domain “b*” is complementary to sequence domain “b”, enabling hybridization to form a 1^{st} duplex. In FIG. 14A, the 1^{st} duplex may be also described as hybridization between the 1^{st} segment and the 7^{th} segment (in this example, the 1^{st} segment corresponds to sequence domain “b” and the 7^{th} segment corresponds to sequence domain “b*”). In some settings it is convenient to designate complementary sequence domains using matching domain names with and without an asterisk (for example, domain “b*” complementary to domain “b”). Complementarity may also be specified independent of the sequence domain names. For example, domain “b” may be specified as complementary to domain “c”. The complementarity between two complementary sequence domains may be partial, such that when they base-pair to each other to form a duplex, the base pairs within the duplex may have one or more mismatches interspersed between them (i.e., one or more unpaired bases interspersed between the base pairs within the duplex). In some embodiments, a duplex consists of 1, 2, 3, 4, 5, 6, 7, 8, 9, 10, 11, 12, 13, 14, 15, 16, 17, 18, 19, 20 or more consecutive base pairs between two segments. In some embodiments a duplex consists of 5, 10, 15, 20, 25, 30, 35, 40, 45, or 50 consecutive base pairs (or any integer number of consecutive base pairs in between any of these values) between two segments. In some embodiments a duplex consists of 10, 20, 30, 40, 50, 60, 70, 80, 90, or 100 consecutive base pairs (or any integer number of consecutive base pairs in between any of these values) between two segments). In some embodiments a duplex consists of 100, 200, 300, 400, or 500 consecutive base pairs (or any integer number of consecutive base pairs in between any of these values) between two segments. In some embodiments, a duplex consists of 2, 3, 4, 5, 6, 7, 8, 9, 10, 11, 12, 13, 14, 15, 16, 17, 18, 19, 20 or more base pairs between two segments wherein 1, 2, 3, 4, 5, 6, 7, 8, 9, 10, 11, 12, 13, 14, 15, 16, 17, 18, 19, 20 or more unpaired bases are interspersed at one or more locations between the base pairs. In some embodiments a duplex consists of 5, 10, 15, 20, 25, 30, 35, 40, 45, or 50 base pairs (or any integer number of base pairs in between any of these values) between two segments wherein 1, 5, 10, 15, 20, 25, 30, 35, 40, 45, or 40 unpaired bases (or any integer number of unpaired bases between any of these values) are interspersed at one or more locations between the base pairs. In some embodiments a duplex consists of 10, 20, 30, 40, 50, 60, 70, 80, 90, or 100 base pairs (or any integer number of base pairs in between any of these values) between two segments wherein 1, 10, 20, 30, 40, 50, 60, 70, 80, 90, or 100 unpaired bases (or any integer number of unpaired bases between any of these values) are interspersed at one or more locations between the base pairs. In some embodiments a duplex consists of 100, 200, 300, 400, or 500 base pairs (or any integer number of base pairs in between any of these values) between two segments wherein 1, 100, 200, 300, 400, or 500 unpaired bases (or any integer number of unpaired bases between any of these values) are interspersed at one or more locations between the base pairs. In some embodiments, a duplex comprising N base pairs between 2 segments further comprises 1, 2, 3, 4, 5, 6, 7, 8, 9, 10 or more mismatches corresponding to bases that are unpaired. In some embodiments, a duplex comprising N base pairs between 2 segments further comprises 0% N, 1% N, 2% N, 5% N, 10% N, 20% N, 50% N, 100% N, or 200% N or more mismatches (or any percentage of N mismatches intermediate to the stated values) corresponding to bases that are unpaired.

A nucleic acid secondary structure can be depicted as a “polymer graph” in which the segments comprising the strand are depicted 5′ to 3′ along a straight backbone and each duplex (corresponding to base-pairing between segments) is depicted as an arc. For example, FIG. 14B depicts the polymer graph corresponding to the secondary structure of FIG. 14A; the 1^{st} segment hybridizes to the 7^{th} segment to form a 1^{st} duplex, the 2^{nd} segment hybridizes to the 3^{rd} segment to form a 2^{nd} duplex, the 4^{th} segment hybridizes to the 6^{th} segment for form a 3^{rd} duplex, and the 5^{th} segment hybridizes to the 8^{th} segment to form a 4^{th} duplex.

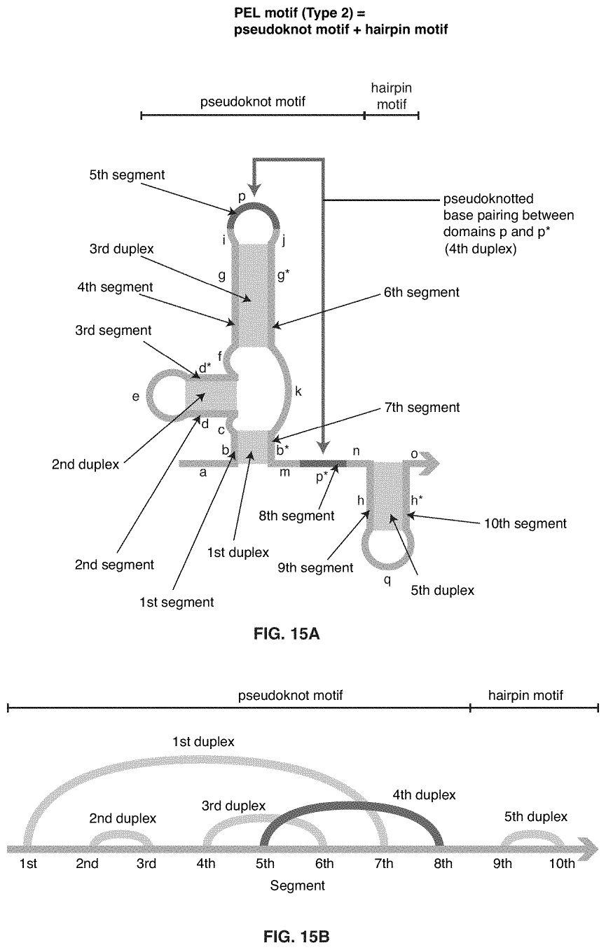

A secondary structure is “pseudoknotted” (i.e., comprises a “pseudoknot”) if the corresponding polymer graph representation contains crossing arcs; a secondary structure is “unpseudoknotted” (i.e., comprises no “pseudoknots”) if it contains no crossing arcs. For example, the secondary structure of FIG. 14A is pseudoknotted because the polymer graph of FIG. 14B contains crossing arcs; we refer to the secondary structure of FIG. 14A as “pseudoknot motif” because it comprises a pseudoknot. In some embodiments, the backbone can be subdivided into multiple motifs, some of which are pseudoknotted and some of which are not. For example, FIG. 15a depicts a secondary structure with a pseudoknot motif at the 5′ end (comprising the 1^{st}-8^{th} segments) and a hairpin (unpsueodoknotted) motif at the 3′ end (comprising the 9^{th} and 10^{th} segments). In the corresponding polymer graph of FIG. 15B, the pseudoknot motif comprising the 1^{st}-8^{th} segments has crossing arcs while the hairpin (unpseudknotted) motif comprising the 9^{th} and 10^{th} segments does not have cross arcs. A “hairpin motif” comprises a hairpin structure in which a strand folds back on itself and base pairs to itself to create a hairpin loop (comprising 3 or more unpaired nucleotides) closed by a duplex, optionally including additional unpaired nucleotides at the 5′ and/or 3′ ends of the motif. For example, FIG. 15A depicts a hairpin motif comprising the 5^{th} duplex (formed by hybridization between the 9^{th} and 10^{th} segments; equivalently by base-pairing between sequence domains “h” and “h*”) and the hairpin loop comprising the unpaired bases of sequence domain “q”.

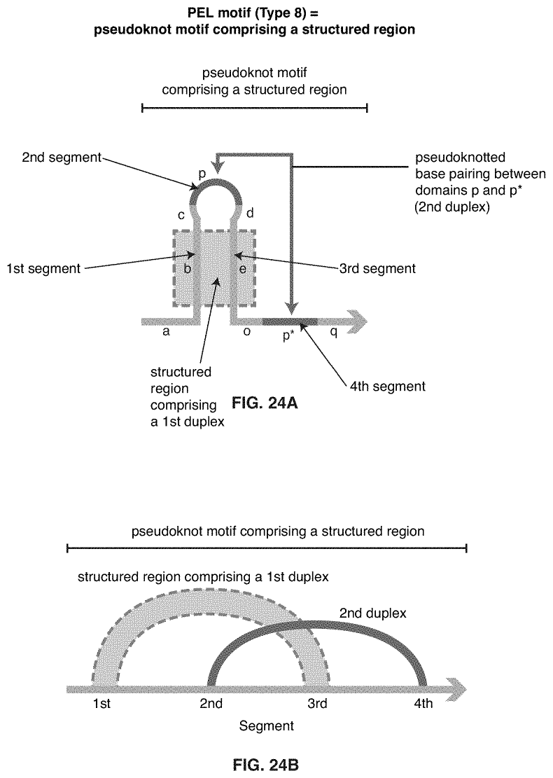

Within a secondary structure, we use the term “structured region” to refer to a region comprising one or more base pairs. For example, FIG. 24A depicts: 1) a 1^{st} segment that hybridizes to a 3^{rd} segment to form a structured region comprising a 1^{st} duplex (wherein the structured region additionally comprises none, some, or all of: a) one or more intra-segment base pairs within the 1^{st} segment, b) one or more intra-segment base pairs within the 3^{rd} segment, c) a combination of intra-segment and inter-segment base pairs within and between the 1^{st} and 3^{rd} segments), and 2) a 2^{nd} segment that hybridizes to a 4^{th} segment to form a 2^{nd} duplex. FIG. 24B depicts the corresponding polymer graph in which the segments are depicted 5′ to 3′ along a straight backbone, the structured region comprising a 1^{st} duplex is depicted as a light gray arc with a dashed boundary, and the 2^{nd} duplex is depicted as a dark gray arc. In the polymer graph of FIG. 24B, the arc denoting the structured region comprising a 1^{st} duplex crosses the arc denoting the 2^{nd} duplex, indicating that the secondary structure is pseudoknotted (i.e., that FIGS. 24A and 24B denote a pseudoknot motif).

As used herein, the term “exoribonuclease-resistant RNA (xrRNA)” denotes a portion of a viral RNA that forms a mechanical block to halt exoribonucleases and inhibit RNA degradation.

As used herein, the term “reduces degradation” (for example, of a “protected nucleic acid”) means any of the following equivalent statements: 1) increases the duration of time during which the protected nucleic acid remains intact and capable of performing its intended function, 2) increases the population, at any given time point, of protected nucleic acid molecules that have not been enzymatically broken up into small non-functional fragments, 3) slows down the process of enzymatic destruction of a population of protected nucleic acids, 4) increases the fraction of protected nucleic acids that remain structurally intact and functionally operational and are not cut into molecular components.

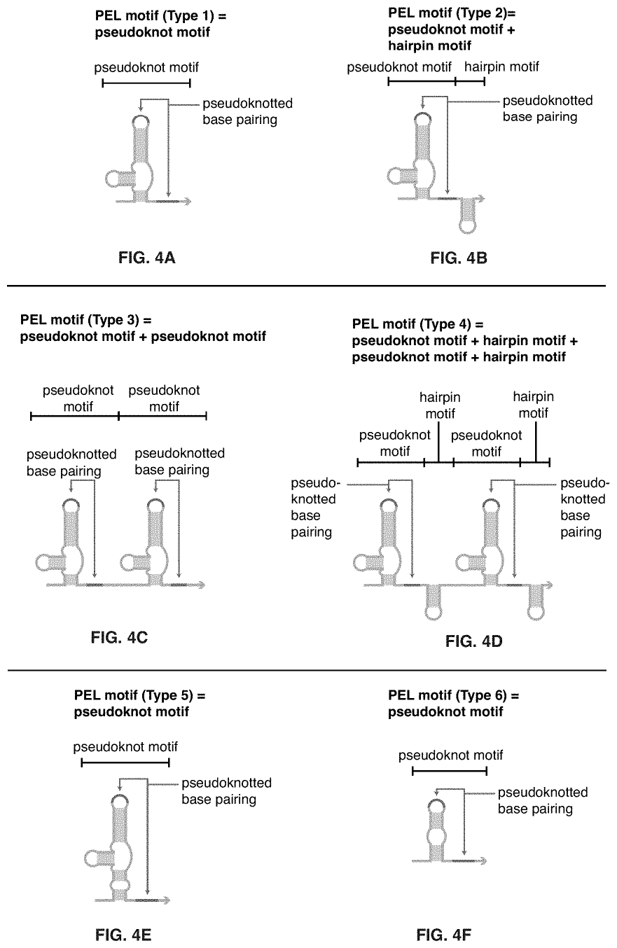

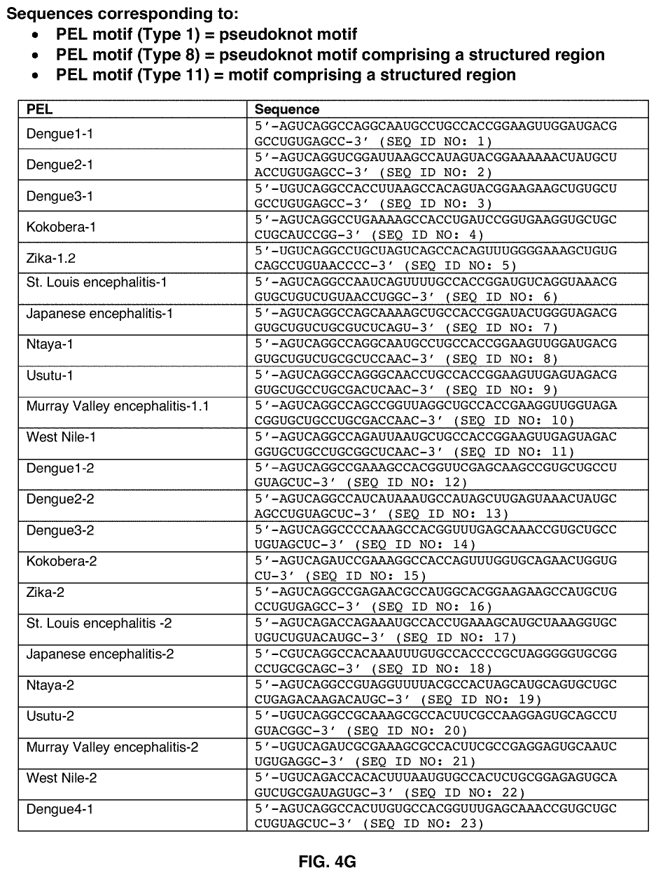

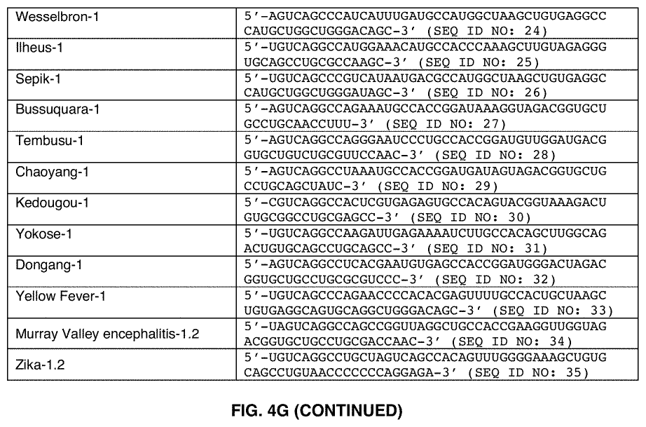

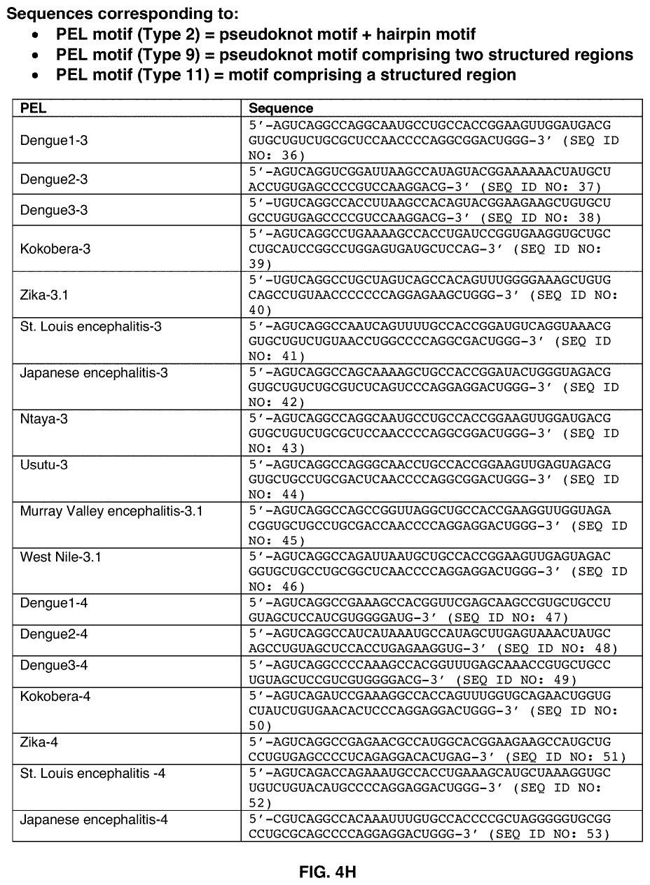

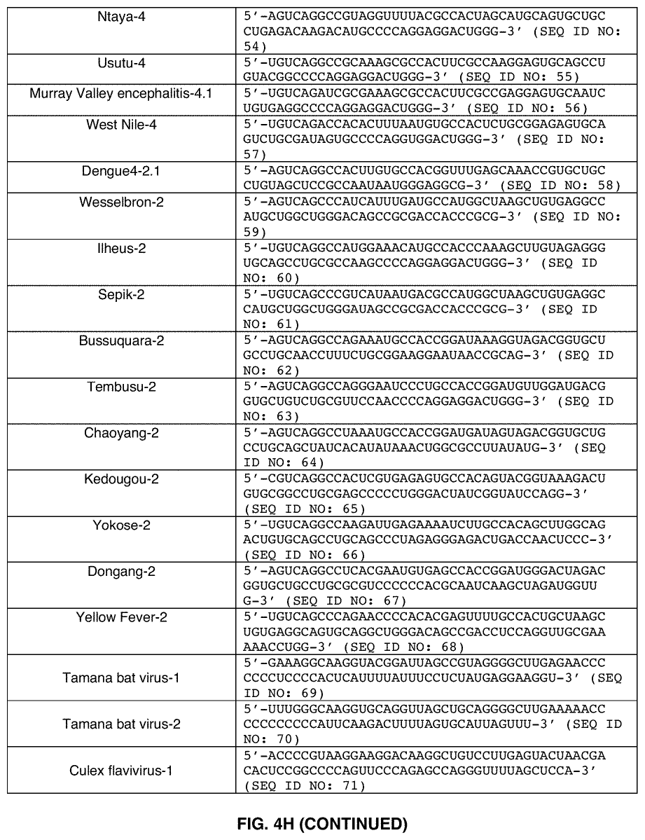

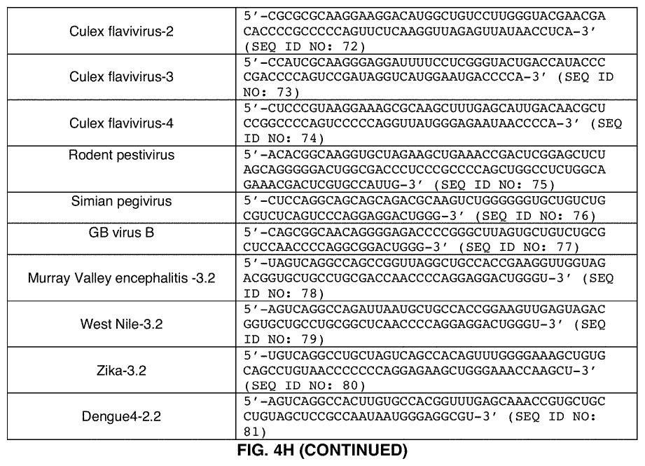

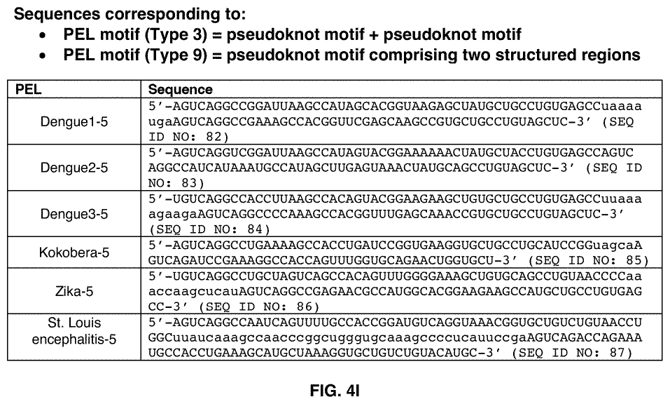

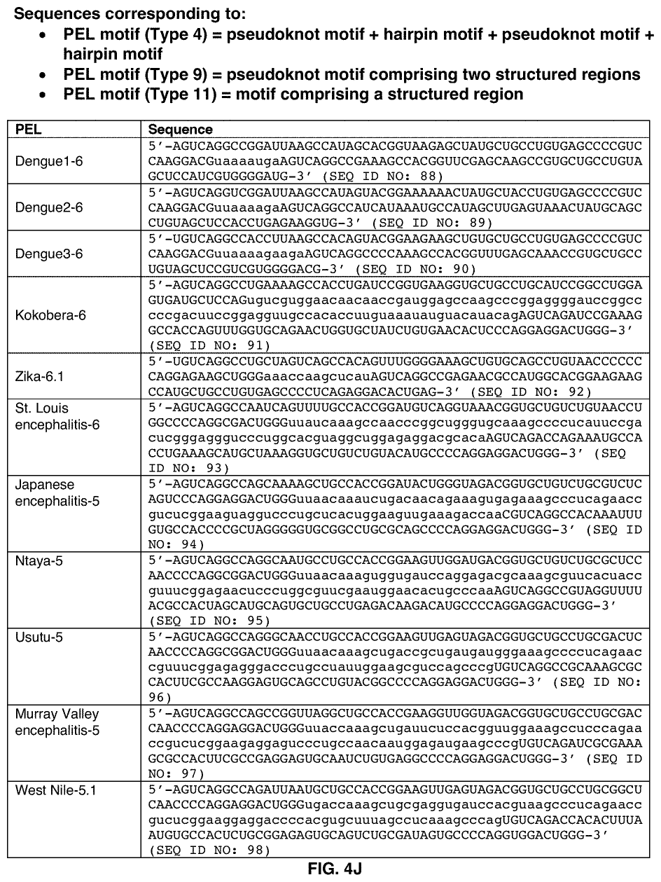

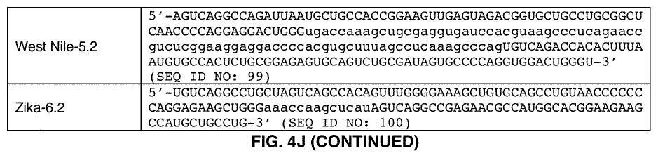

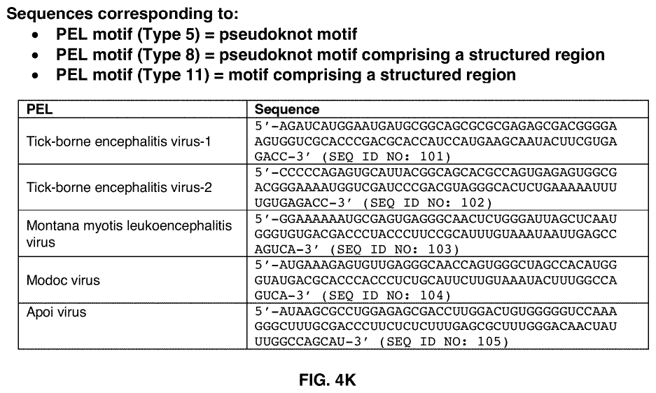

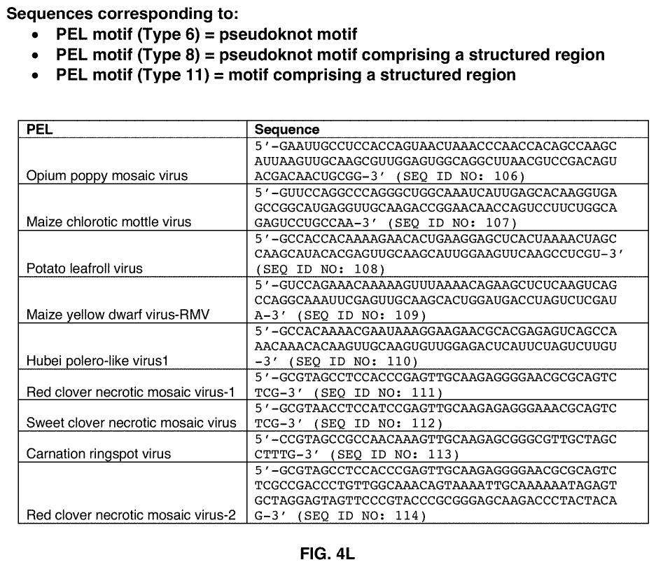

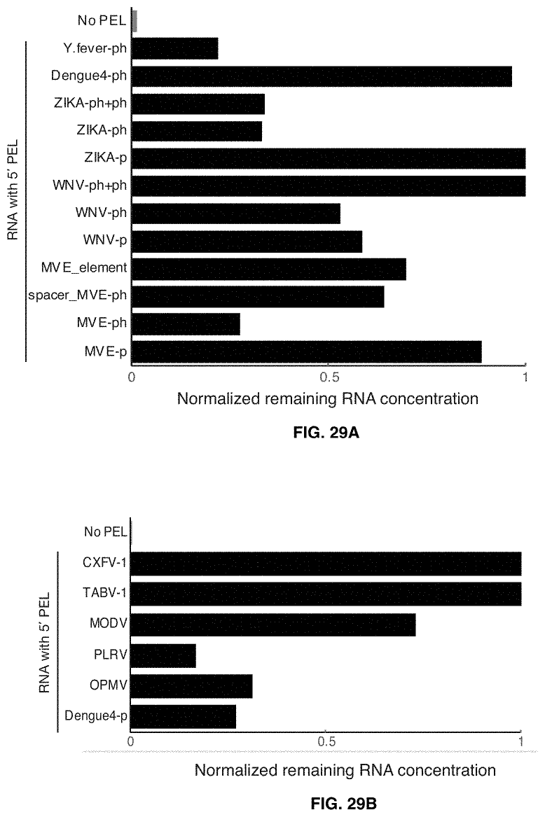

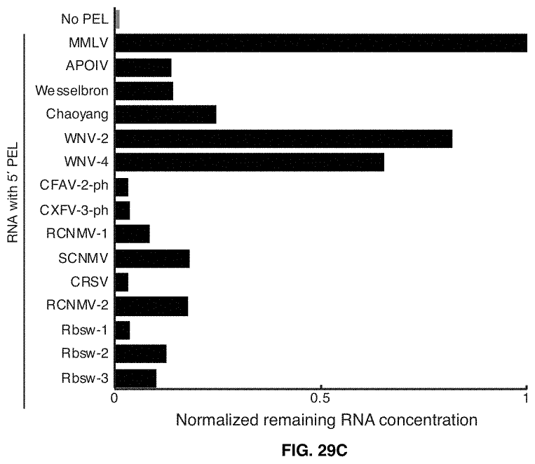

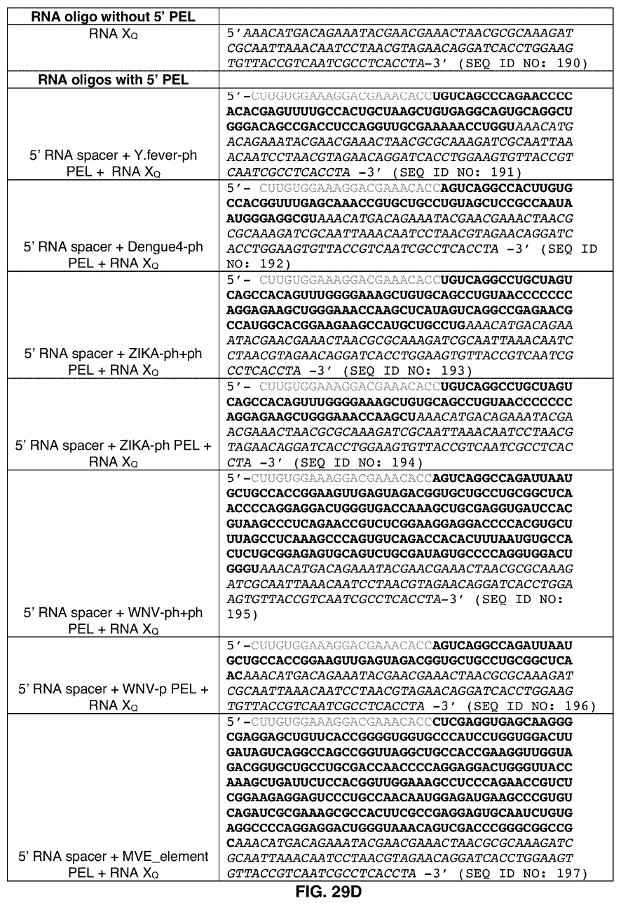

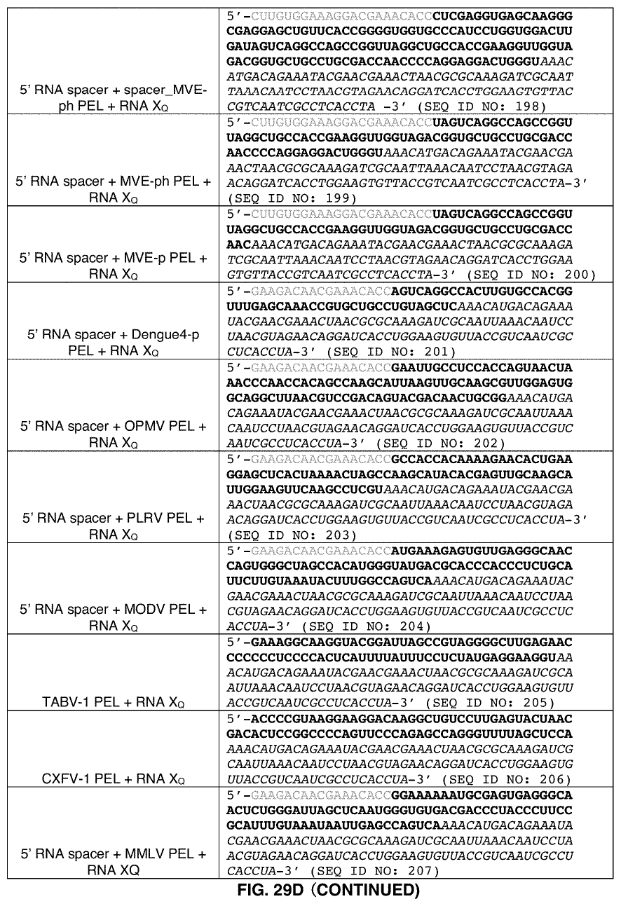

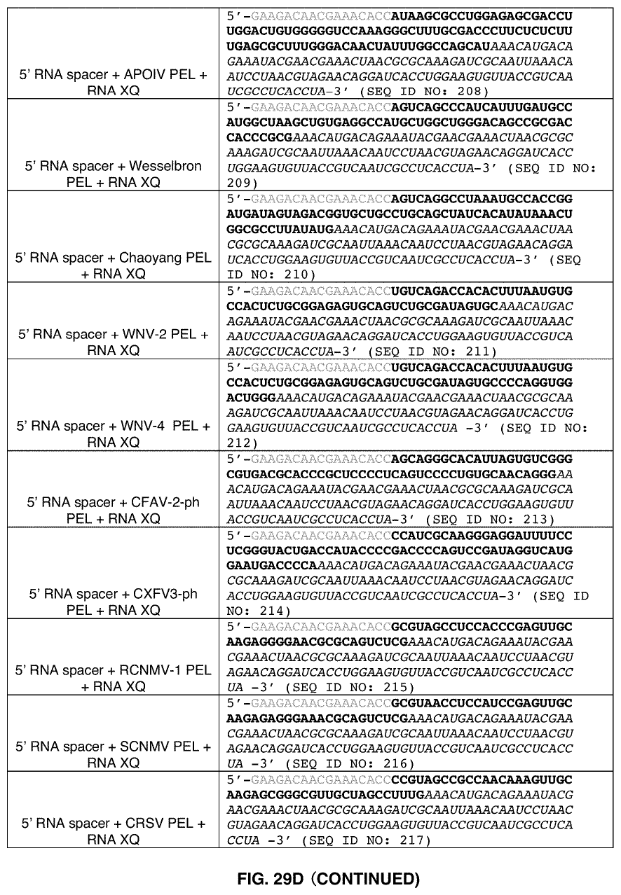

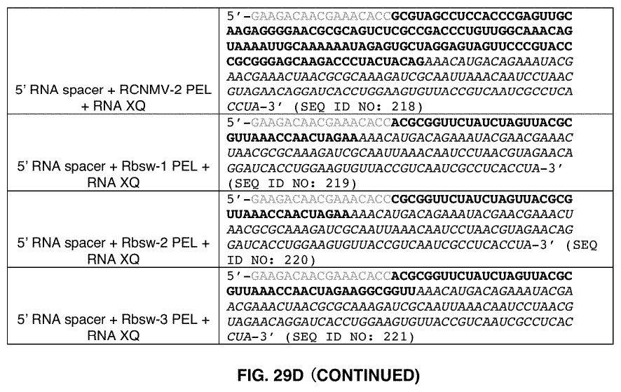

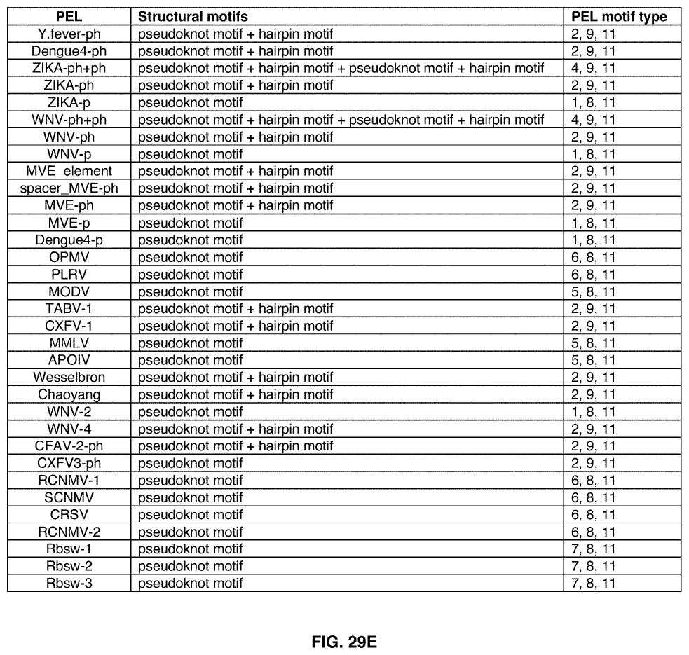

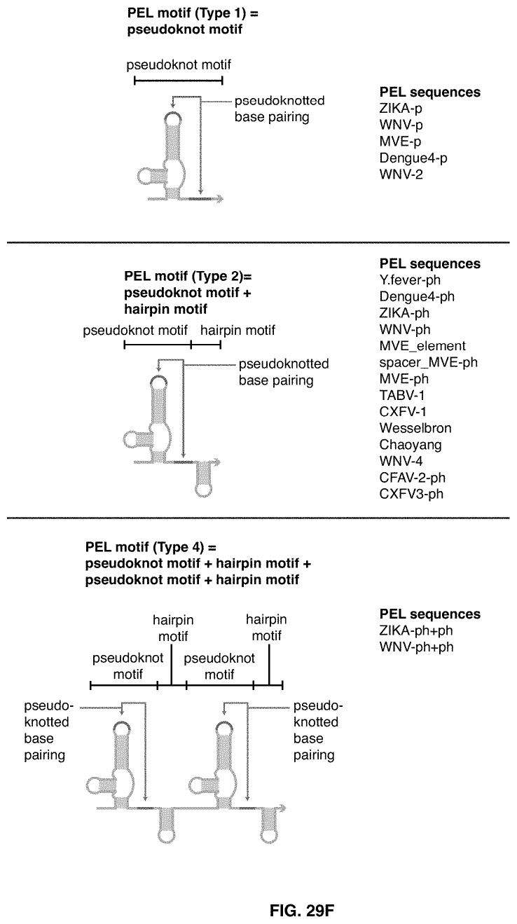

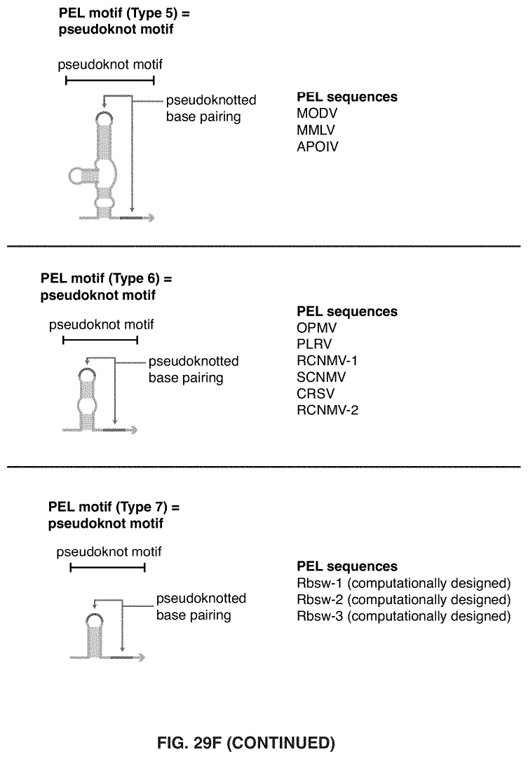

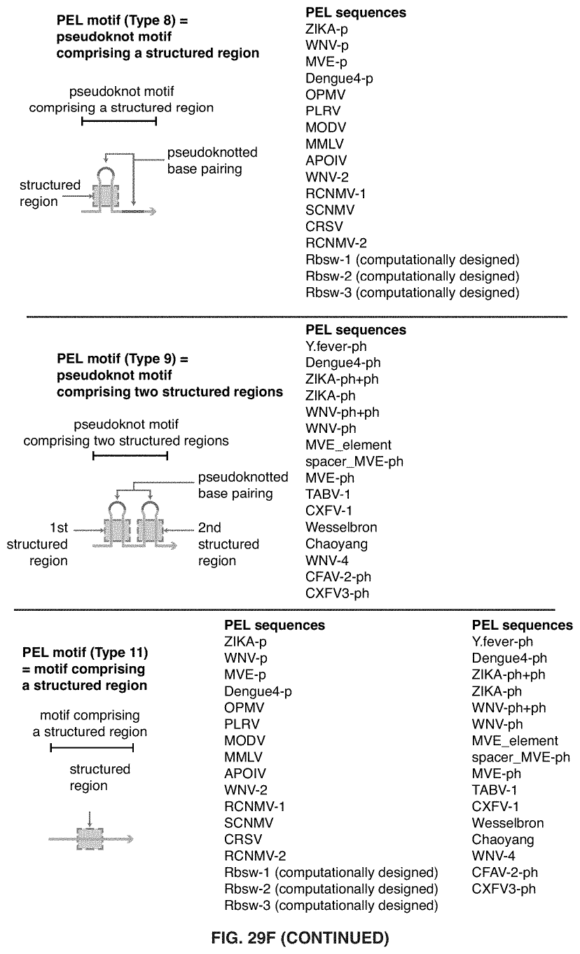

As used herein, the term “protective element (PEL)” denotes a portion of a nucleic acid comprising a structured region that reduces degradation of a protected nucleic acid by nucleases. The term PEL may be used to refer to: 1) the structural motif of the PEL (also known as a “PEL motif”) comprising one or more segments interacting to form one or more duplexes (for example, the PEL motif of FIG. 14A comprising 8 segments interacting to form 4 duplexes), 2) and/or the sequence of the PEL (also known as a “PEL sequence”; for example, the PEL sequences of FIG. 4A). FIG. 29F illustrates examples of PEL motifs and PEL sequences. PELs, PEL motifs, and/or PEL sequences can be: 1) derived from xrRNAs, 2) rationally designed, 3) engineered by directed evolution, 4) obtained from any combination of the above.

As used herein, “combining” encompasses any act or situation where at least two elements are able to interact, including, for example, adding one to the other, allowing the two elements to interact, exposing the two elements to each other, placing or having arranged the elements in a situation where they can interact, etc.

As used herein, the term “providing” encompasses any way to provide the denoted material, including for example, having, obtaining, creating, causing to be created, suppling, etc. the denoted material. This can be done directly (such as the provision of an RNA molecule itself) or indirectly (such as the provision of an DNA molecule that is to be transcribed into the RNA molecule). In some embodiments, this process can be an independent process (such as by obtaining an RNA segment), or it can be part of another process in the method (such as by providing an DNA sequence that is then transcribed into an RNA sequence).

As used in some embodiments herein, the term “mediating” can include one or more of facilitating, directing, or enabling.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive. Unless defined otherwise, technical and scientific terms used herein have the same meaning as commonly understood by one of ordinary skill in the art to which this invention belongs. See, for example Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). It is to be understood that both the general description and the detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

### PEL Sequences and Structural Motifs

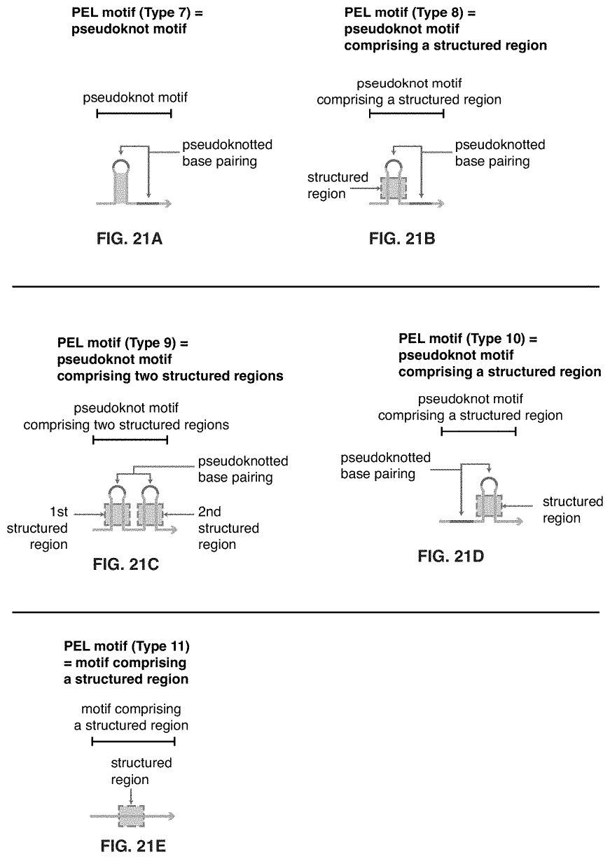

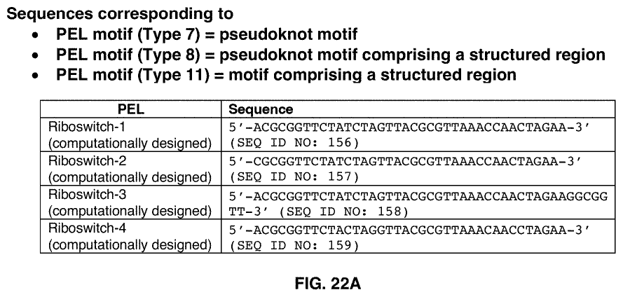

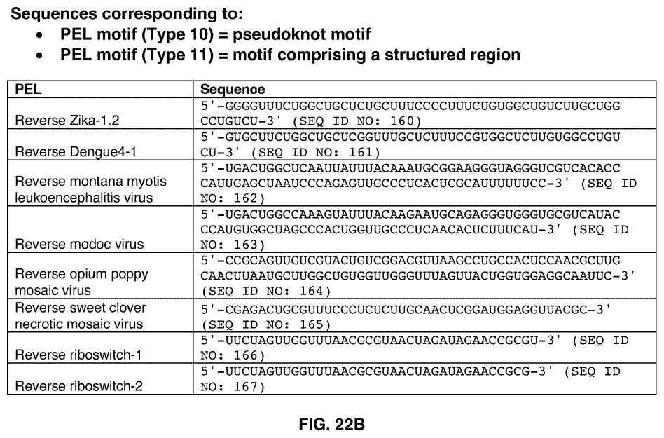

Viruses protect against degradation using exoribonuclease-resistant RNA (xrRNA) motifs that form a mechanical block to halt diverse 5′ exoribonucleases.¹⁻⁹ In some embodiments, the present invention uses protective elements (PELs) to reduce nucleic acid degradation for synthetic biology. In some embodiments, PELs enhance the performance of nucleic acid synthetic biology. In some embodiments, PELs are derived from viral xrRNAs. In some embodiments, a PEL comprises 1%, 2%, 5%, 10%, 20%, 30%, 40%, 50%, 60%, 70%, 80%, 90%, or 100% of a viral xrRNA. In some embodiments, a PEL comprises a pseudoknot motif (for example, FIG. 4A, FIG. 4E, FIG. 4F, FIG. 21A, FIG. 21B, FIG. 21C, or FIG. 21D). In some embodiments, a PEL comprises a hairpin motif (for example, FIG. 21E with the structured region comprising a hairpin motif). In some embodiments, a PEL comprises a pseudoknot motif in conjunction with a hairpin motif (for example, FIG. 4B). In some embodiments, a PEL comprises one or more pseudoknot motifs (for example, FIG. 4C displays a PEL comprising a first pseudoknot motif and a second pseudoknot motif). In some embodiments, a PEL comprises one or more hairpin motifs (for example, FIG. 21E with the structured region comprising one or more hairpin motifs). In some embodiments, a PEL comprises one or more pseudoknot motifs and one or more hairpin motifs (for example, FIG. 4D displays a PEL comprising a first pseudoknot motif, a first hairpin motif, a second pseudoknot motif, and a second hairpin motif). In some embodiments, a PEL comprises multiple segments derived from the same xrRNA and/or from different xrRNAs. In some embodiments, a PEL comprises rationally designed sequences and structural motifs. In some embodiments, a PEL comprises sequences and structural motifs engineered by directed evolution. In some embodiments, a PEL comprises rationally designed sequences and biologically derived structural motifs. In some embodiments, a PEL comprises multiple segments, one or more of which are derived from one or more xrRNAs and one or more of which are rationally designed. In some embodiment, a PEL comprises components that are biologically derived, rationally designed, and/or engineered by directed evolution. FIG. 4G displays examples of PEL sequences for the PEL motif of FIG. 4A (Type 1; comprising a pseudoknot motif). FIG. 4H displays examples of PEL sequences for the PEL motif of FIG. 4B (Type 2; comprising a pseudoknot motif and a hairpin motif). FIG. 4I displays examples of PEL sequences for the PEL motif of FIG. 4C (Type 3; comprising a first pseudoknot motif and a second pseudoknot motif). FIG. 4J displays examples of PEL sequences for the PEL motif of FIG. 4D (Type 4; comprising a first pseudoknot motif, a first hairpin motif, a second pseudoknot motif, and a second hairpin motif). FIG. 4K displays examples of PEL sequences for PEL motif of FIG. 4E (Type 5; comprising a pseudoknot motif). FIG. 4L displays examples of PEL sequences for the PEL motif of FIG. 4F (Type 6; comprising a pseudoknot motif). FIG. 22A displays examples of PEL sequences for the PEL motif of FIG. 21A (Type 7; comprising a pseudoknot motif). FIGS. 4G, 4K, 4L and 22A display examples of PEL sequences for the PEL motif of FIG. 21B (Type 8; comprising a pseudoknot motif comprising a structured region). FIGS. 4H-4J display examples of PEL sequences for the PEL motif of FIG. 21C (Type 9; comprising a pseudoknot motif comprising two structured regions). FIG. 22B displays examples of PEL sequences for the PEL motif of FIG. 21D (Type 10; comprising a pseudoknot motif comprising a structured region). FIGS. 4G-4L and 22A-22B display examples of PEL sequences for the PEL motif of FIG. 21E (Type 11; comprising a structured region). In some embodiments, for any of the PELs, or method of making, or use provided herein, the PEL can comprise, consist or consist essentially of an exoribonuclease-resistant RNA (xrRNA).

### PEL Motifs (Type 1) Comprising Pseudoknot Motif

In some embodiments, a PEL motif comprises a pseudoknot motif (see for example the secondary structure schematic of FIG. 14A). In some embodiments, the pseudoknot motif comprises (from 5′ to 3′) a 1^{st} segment, a 2^{nd} segment, a 3^{rd} segment, a 4^{th} segment, a 5^{th} segment, a 6^{th} segment, a 7^{th} segment, and an 8^{th} segment. In some embodiments, the 1^{st} segment hybridizes to the 7^{th} segment to form a 1^{st} duplex, the 2^{nd} segment hybridizes to the 3^{rd} segment to form a 2^{nd} duplex, the 4^{th} segment hybridizes to the 6^{th} segment to form a 3^{rd} duplex, and the 5^{th} segment hybridizes to the 8^{th} segment to form a 4^{th} duplex. These relationships between segments and duplexes are depicted in the secondary structure schematic of FIG. 14A and in the polymer graph schematic of FIG. 14B (in which the segments are depicted 5′ to 3′ along the straight backbone and each duplex is depicted as an arc). In some embodiments, a duplex comprises a set of 1 or more base pairs. In some embodiments, a duplex comprises a set of 2 or more base pairs. In some embodiments, a duplex comprises a set of 2 or more consecutive base pairs. In some embodiments, the 1^{st} segment corresponds to domain “b”, the 2^{nd} segment corresponds to domain “d”, the 3^{rd} segment corresponds to domain “d*”, the 4^{th} segment corresponds to domain “g”, the 5^{th} segment corresponds to domain “p”, the 6^{th} segment corresponds to domain “g*”, the 7^{th} segment corresponds to domain “b*”, and the 8^{th} segment corresponds to domain “p*”. In some embodiments, the 1^{st} duplex corresponds to base-pairing between domains “b” and “b*”, the 2^{nd} duplex corresponds to base-pairing between domains “d” and “d*”, the 3^{rd} duplex corresponds to base-pairing between domains “g” and “g*”, and the 4^{th} duplex corresponds to base-pairing between domains “p” and “p*”. In some embodiments, there are 0, 1, 2, 3 or more unpaired bases 5′ or 3′ of any of the numbered segments (these unpaired bases are also known as domains “a”, “c”, “e”, “f”, “i”, “j”, “k”, “m”, and “n”; see FIG. 14A). In some embodiments, some or all of domains “a”, “c”, “e”, “f”, “i”, “j”, “k”, “m”, and “n” form intra-domain or inter-domain base pairs. In some embodiments, an additional duplex forms between bases 5′ of the 1^{st} segment (also known as domain “a”; see FIG. 14A) and bases 3′ of the 6^{th} segment and 5′ of the 7^{th} segment (also known as domain “k”; see FIG. 14A). In some embodiments, additional base-pairing and/or tertiary contacts form within the PEL motif. In some embodiments, the PEL motif serves as a mechanical block to prevent nuclease degradation of a nucleic acid comprising the PEL motif.

### PEL Motifs (Type 2) Comprising a Pseudoknot and a Hairpin Motif

In some embodiments, a PEL motif comprises (from 5′ to 3′) a pseudoknot motif and a hairpin motif (see for example the secondary structure schematic of FIG. 15A). In some embodiments, the pseudoknot motif comprises (from 5′ to 3′) a 1^{st} segment, a 2nd segment, a 3^{rd} segment, a 4^{th} segment, a 5^{th} segment, a 6^{th} segment, a 7^{th} segment, and an 8^{th} segment. In some embodiments, the 1^{st} segment hybridizes to the 7^{th} segment to form a 1^{st} duplex, the 2^{nd} segment hybridizes to the 3^{rd} segment to form a 2^{nd} duplex, the 4^{th} segment hybridizes to the 6^{th} segment to form a 3^{rd} duplex, the 5^{th} segment hybridizes to the 8^{th} segment to form a 4^{th} duplex. In some embodiments, the hairpin motif comprises (from 5′ to 3′) a 9^{th} segment and a 10^{th} segment. In some embodiments, the 9^{th} segment hybridizes to the 10^{th} segment to form a 5^{th} duplex. These relationships between segments and duplexes are depicted in the secondary structure schematic of FIG. 15A and in the polymer graph schematic of FIG. 15B (in which the segments are depicted 5′ to 3′ along the straight backbone and each duplex is depicted as an arc). In some embodiments, a duplex comprises a set of 1 or more base pairs. In some embodiments, a duplex comprises a set of 2 or more base pairs. In some embodiments, a duplex comprises a set of 2 or more consecutive base pairs. In some embodiments, the 1^{st} segment corresponds to domain “b”, the 2^{nd} segment corresponds to domain “d”, the 3^{rd} segment corresponds to domain “d*”, the 4^{th} segment corresponds to domain “g”, the 5^{th} segment corresponds to domain “p”, the 6^{th} segment corresponds to domain “g*”, the 7^{th} segment corresponds to domain “b*”, the 8^{th} segment corresponds to domain “p*”, the 9^{th} segment corresponds to domain “h”, and the 10^{th} segment corresponds to domain “h*”. In some embodiments, the 1^{st} duplex corresponds to base-pairing between domains “b” and “b*”, the 2′ duplex corresponds to base-pairing between domains “d” and “d*”, the 3^{rd} duplex corresponds to base-pairing between domains “g” and “g*”, the 4^{th} duplex corresponds to base-pairing between domains “p” and “p*”, and the 5^{th} duplex corresponds to base-pairing between domains “h” and “h*”. In some embodiments, there are 0, 1, 2, 3 or more unpaired bases 5′ or 3′ of the any of the above segments (these unpaired bases are also known as domains “a”, “c”, “e”, “f”, “i”, “j”, “k”, “m”, “n”, “q”, and “o”; see FIG. 15A). In some embodiments, some or all of domains “a”, “c”, “e”, “f”, “i”, “j”, “k”, “m”, “n”, “q”, and “o” form intra-domain or inter-domain base pairs. In some embodiments, an additional duplex forms between bases 5′ of the 1^{st} segment (also known as domain “a”; see FIG. 15A) and bases that are 3′ of the 6^{th} segment and 5′ of the 7^{th} segment (also known as domain “k”; see FIG. 15A). In some embodiments, additional base-pairing and/or tertiary contacts form within the PEL motif. In some embodiments, the pseudoknot motif and the hairpin motif are connected by a linker of zero, one, two or more nucleotides. In some embodiments, the pseudoknot motif and the hairpin motif are connected by a chemical linker that is not capable of base-pairing. In some embodiments, the PEL motif serves as a mechanical block to prevent nuclease degradation of a nucleic acid comprising the PEL motif.

### PEL Motifs (Type 3) Comprising a First Pseudoknot Motif and Second Pseudoknot Motif

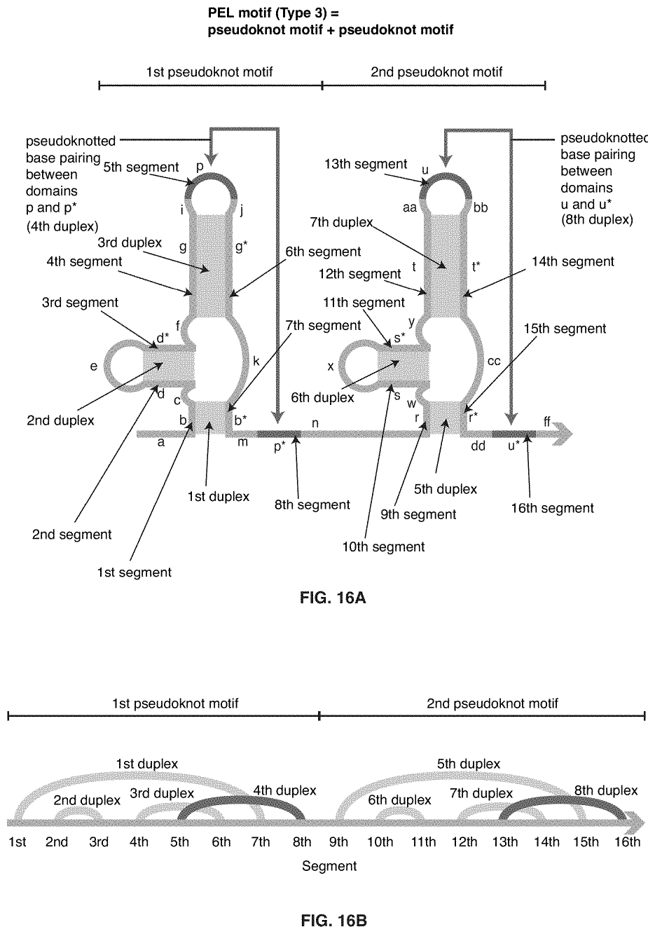

In some embodiments, a PEL motif comprises (from 5′ to 3′) a first pseudoknot motif and a second pseudoknot motif (see for example the secondary structure schematic of FIG. 16A). In some embodiments, the first pseudoknot motif comprises (from 5′ to 3′) a 1^{st} segment, a 2^{nd} segment, a 3^{rd} segment, a 4^{th} segment, a 5^{th} segment, a 6^{th} segment, a 7^{th} segment, and an 8^{th} segment. In some embodiments, the 1^{st} segment hybridizes to the 7^{th} segment to form a 1^{st} duplex, the 2^{nd} segment hybridizes to the 3^{rd} segment to form a 2^{nd} duplex, the 4^{th} segment hybridizes to the 6^{th} segment to form a 3^{rd} duplex, and the 5^{th} segment hybridizes to the 8^{th} segment to form a 4^{th} duplex. In some embodiments, the second pseudoknot motif comprises (from 5′ to 3′) a 9^{th} segment, a 10^{th} segment, an 11^{th} segment, a 12^{th} segment, a 13^{th} segment, a 14^{th} segment, a 15^{th} segment, and a 16^{th} segment. In some embodiments, the 9^{th} segment hybridizes to the 15^{th} segment to form a 5^{th} duplex, the 10^{th} segment hybridizes to the 11^{th} segment to form a 6^{th} duplex, the 12^{th} segment hybridizes to the 14^{th} segment to form a 7^{th} duplex, and the 13^{th} segment hybridizes to the 16^{th} segment to form an 8^{th} duplex. These relationships between segments and duplexes are depicted in the secondary structure schematic of FIG. 16A and in the polymer graph schematic of FIG. 16B (in which the segments are depicted 5′ to 3′ along the straight backbone and each duplex is depicted as an arc). In some embodiments, a duplex comprises a set of 1 or more base pairs. In some embodiments, a duplex comprises a set of 2 or more base pairs. In some embodiments, a duplex comprises a set of 2 or more consecutive base pairs. In some embodiments, the 1^{st} segment corresponds to domain “b”, the 2^{nd} segment corresponds to domain “d”, the 3^{rd} segment corresponds to domain “d*”, the 4^{th} segment corresponds to domain “g”, the 5^{th} segment corresponds to domain “p”, the 6^{th} segment corresponds to domain “g*”, the 7^{th} segment corresponds to domain “b*”, the 8^{th} segment corresponds to domain “p*”, the 9^{th} segment corresponds to domain “r”, and the 10^{th} segment corresponds to domain “s”, the 11^{th} segment corresponds to domain “s*”, the 12^{th} segment corresponds to domain “t”, the 13^{th} segment corresponds to domain “u”, the 14^{th} segment corresponds to domain “t*”, the 15^{th} segment corresponds to domain “r*”, and the 16^{th} segment corresponds to domain “u*”. In some embodiments, the 1^{st} duplex corresponds to base-pairing between domains “b” and “b*”, the 2^{nd} duplex corresponds to base-pairing between domains “d” and “d*”, the 3^{rd} duplex corresponds to base-pairing between domains “g” and “g*”, the 4^{th} duplex corresponds to base-pairing between domains “p” and “p*”, the 5^{th} duplex corresponds to base-pairing between domains “r” and “r*”, the 6^{th} duplex corresponds to base-pairing between domains “s” and “s*”, the 7^{th} duplex corresponds to base-pairing between domains “t” and “t*”, and the 8^{th} duplex corresponds to base-pairing between domains “u” and “u*”. In some embodiments, there are 0, 1, 2, 3 or more unpaired bases 5′ or 3′ of the any of the numbered segments (these unpaired bases are also known as domains “a”, “c”, “e”, “f”, “i”, “j”, “k”, “m”, “n”, “w”, “x”, “y”, “aa”, “bb”, “cc”, “dd”, “ff”; see FIG. 16A). In some embodiments, some or all of domains “a”, “c”, “e”, “f”, “i”, “j”, “k”, “m”, “n”, “w”, “x”, “y”, “aa”, “bb”, “cc”, “dd”, “ff” form intra-domain or inter-domain base pairs. In some embodiments, an additional duplex forms between bases 5′ of the 1^{st} segment (also known as domain “a”) and bases 3′ of the 6^{th} segment and 5′ of the 7^{th} segment (also known as domain “k”; see FIG. 16A). In some embodiments, an additional duplex forms between bases 5′ of the 9^{th} segment (also known as domain “n”) and bases 3′ of the 14^{th} segment and 5′ of the 15^{th} segment (also known as domain “cc”; see FIG. 16A). In some embodiments, additional base-pairing and/or tertiary contacts form within the PEL motif. In some embodiments, the first pseudoknot motif and the second pseudoknot motif are connected by a linker of zero, one, two or more nucleotides. In some embodiments, the first pseudoknot motif and the second pseudoknot motif are connected by a chemical linker that is not capable of base-pairing. In some embodiments, the PEL motif serves as a mechanical block to prevent nuclease degradation of a nucleic acid comprising the PEL motif.

### PEL Motif (Type 4) Comprising a First Pseudoknot Motif, First Hairpin Motif, a Second Pseudoknot Motif, and a Second Hairpin Motif

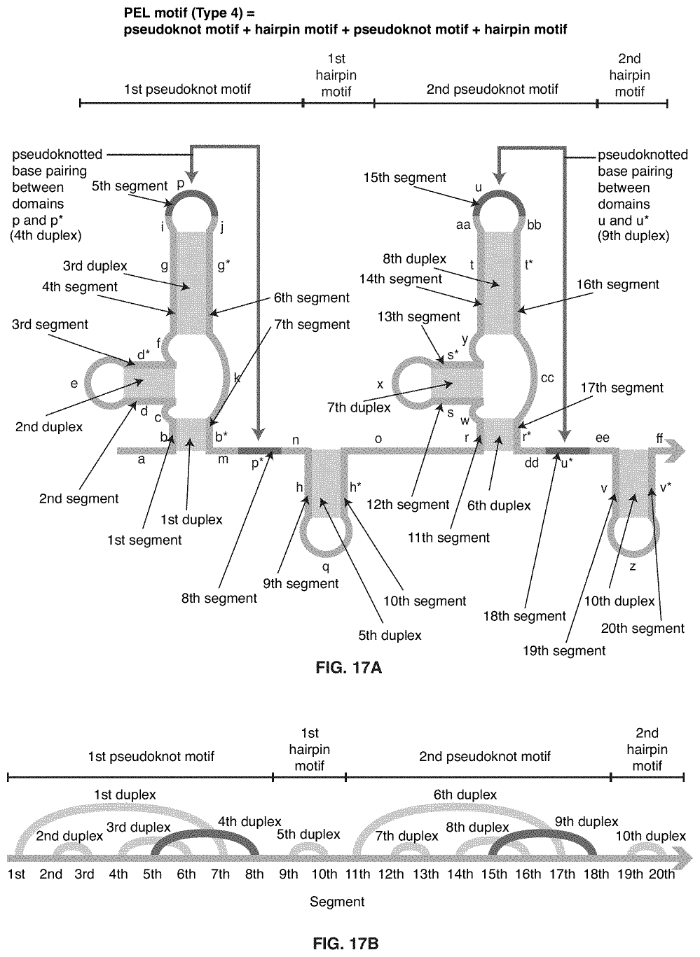

In some embodiments, a PEL motif comprises (from 5′ to 3′) a first pseudoknot motif, a first hairpin motif, a second pseudoknot motif, and a second hairpin motif (see for example the secondary structure schematic of FIG. 17A). In some embodiments, the first pseudoknot motif comprises (from 5′ to 3′) a 1^{st} segment, a 2^{nd} segment, a 3^{rd} segment, a 4^{th} segment, a 5^{th} segment, a 6^{th} segment, a 7^{th} segment, and an 8^{th} segment. In some embodiments, the 1^{st} segment hybridizes to the 7^{th} segment to form a 1^{st} duplex, the 2^{nd} segment hybridizes to the 3^{rd} segment to form a 2^{nd} duplex, the 4^{th} segment hybridizes to the 6^{th} segment to form a 3^{rd} duplex, and the 5^{th} segment hybridizes to the 8^{th} segment to form a 4^{th} duplex. In some embodiments, the first hairpin motif comprises (from 5′ to 3′) a 9^{th} segment and a 10^{th} segment. In some embodiments, the 9^{th} segment hybridizes to the 10^{th} segment to form a 5^{th} duplex. In some embodiments, the second pseudoknot motif comprises (from 5′ to 3′) a 11^{th} segment, a 12^{th} segment, a 13^{th} segment, a 14^{th} segment, a 15^{th} segment, a 16^{th} segment, a 17^{th} segment, and an 18^{th} segment. In some embodiments, the 11^{th} segment hybridizes to the 17^{th} segment to form a 6^{th} duplex, the 12^{th} segment hybridizes to the 13^{th} segment to form a 7^{th} duplex, the 14^{th} segment hybridizes to the 16^{th} segment to form an 8^{th} duplex, and the 15^{th} segment hybridizes to the 18^{th} segment to form a 9^{th} duplex. In some embodiments, the second hairpin motif comprises (from 5′ to 3′) a 19^{th} segment and a 20^{th} segment. In some embodiments, the 19^{th} segment hybridizes to the 20^{th} segment to form a 10^{th} duplex. These relationships between segments and duplexes are depicted in the secondary structure schematic of FIG. 17A and in the polymer graph schematic of FIG. 17B (in which the segments are depicted 5′ to 3′ along the straight backbone and each duplex is depicted as an arc). In some embodiments, a duplex comprises a set of 1 or more base pairs. In some embodiments, a duplex comprises a set of 2 or more base pairs. In some embodiments, a duplex comprises a set of 2 or more consecutive base pairs. In some embodiments, the 1^{st} segment corresponds to domain “b”, the 2^{nd} segment corresponds to domain “d”, the 3^{rd} segment corresponds to domain “d*”, the 4^{th} segment corresponds to domain “g”, the 5^{th} segment corresponds to domain “p”, the 6^{th} segment corresponds to domain “g*”, the 7^{th} segment corresponds to domain “b*”, the 8^{th} segment corresponds to domain “p*”, the 9^{th} segment corresponds to domain “h”, and the 10^{th} segment corresponds to domain “h*”, the 11^{th} segment corresponds to domain “r”, the 12^{th} segment corresponds to domain “s”, the 13^{th} segment corresponds to domain “s*”, the 14^{th} segment corresponds to domain “t”, the 15^{th} segment corresponds to domain “u”, the 16^{th} segment corresponds to domain “t*”, the 17^{th} segment corresponds to domain “r*”, the 18^{th} segment correspond to domain “u*”, the 19^{th} segment corresponds to domain “v”, and the 20^{th} segment corresponds to domain “v*”. In some embodiments, the 1^{st} duplex corresponds to base-pairing between domains “b” and “b*”, the 2^{nd} duplex corresponds to base-pairing between domains “d” and “d*”, the 3rd duplex corresponds to base-pairing between domains “g” and “g*”, the 4^{th} duplex corresponds to base-pairing between domains “p” and “p*”, the 5^{th} duplex corresponds to base-pairing between domains “h” and “h*”, the 6^{th} duplex corresponds to base-pairing between domains “r” and “r*”, the 7^{th} duplex corresponds to base-pairing between domains “s” and “s*”, the 8^{th} duplex corresponds to base-pairing between domains “t” and “t*”, the 9^{th} duplex corresponds to base-pairing between domains “u” and “u*”, and the 10^{th} duplex corresponds to base-pairing between domains “v” and “v*”. In some embodiments, there are 0, 1, 2, 3 or more unpaired bases 5′ or 3′ of the any of the above segments (these unpaired bases are also known as domains “a”, “c”, “e”, “f”, “i”, “j”, “k”, “m”, “n”, “q”, “o”, “w”, “x”, “y”, “aa”, “bb”, “cc”, “dd”, “ee”, z″, “ff”; see FIG. 17A). In some embodiments, some or all of domains “a”, “c”, “e”, “f”, “i”, “j”, “k”, “m”, “n”, “q”, “o”, “w”, “x”, “y”, “aa”, “bb”, “cc”, “dd”, “ee”, z″, “ff” form intra-domain or inter-domain base pairs. In some embodiments, an additional duplex forms between bases 5′ of the 1^{st} segment (also known as domain “a”; see FIG. 17A) and bases that are 3′ of the 6^{th} segment and 5′ of the 7^{th} segment (also known as domain “k”; see FIG. 17A). In some embodiments, an additional duplex forms between bases 5′ of the 11^{th} segment (also known as domain “o”; see FIG. 17A) and bases that are 3′ of the 16^{th} segment and 5′ of the 17^{th} segment (also known as domain “cc”; see FIG. 17A). In some embodiments, additional base-pairing and/or tertiary contacts form within the PEL motif. In some embodiments, consecutive motifs within the PEL motif (from 5′ to 3′) are connected by a linker of zero, one, two or more nucleotides. In some embodiments, consecutive motifs within the PEL motif (from 5′ to 3′) are connected by a chemical linker that is not capable of base-pairing. In some embodiments, the PEL motif serves as a mechanical block to prevent nuclease degradation of a nucleic acid comprising the PEL motif.

### PEL Motif (Type 5) Comprising a Pseudoknot Motif

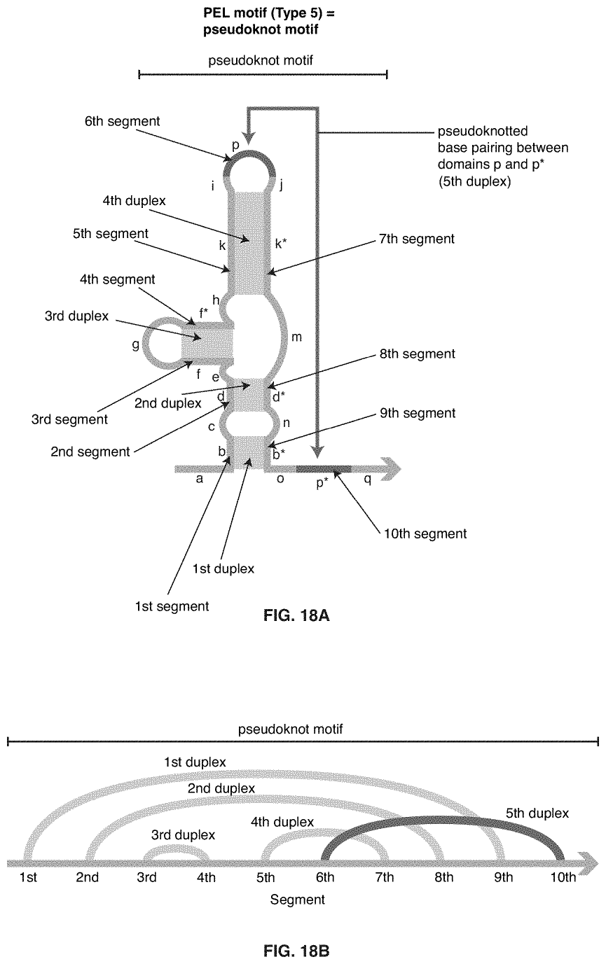

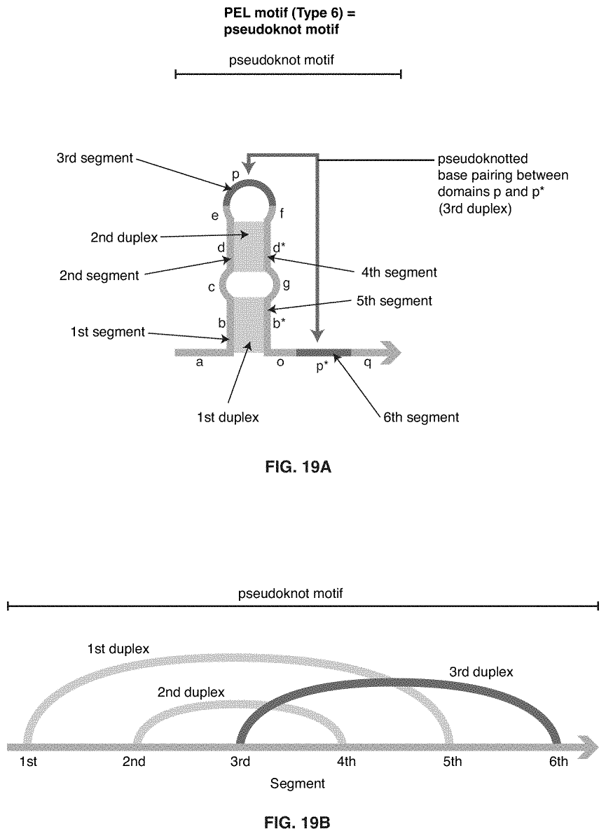

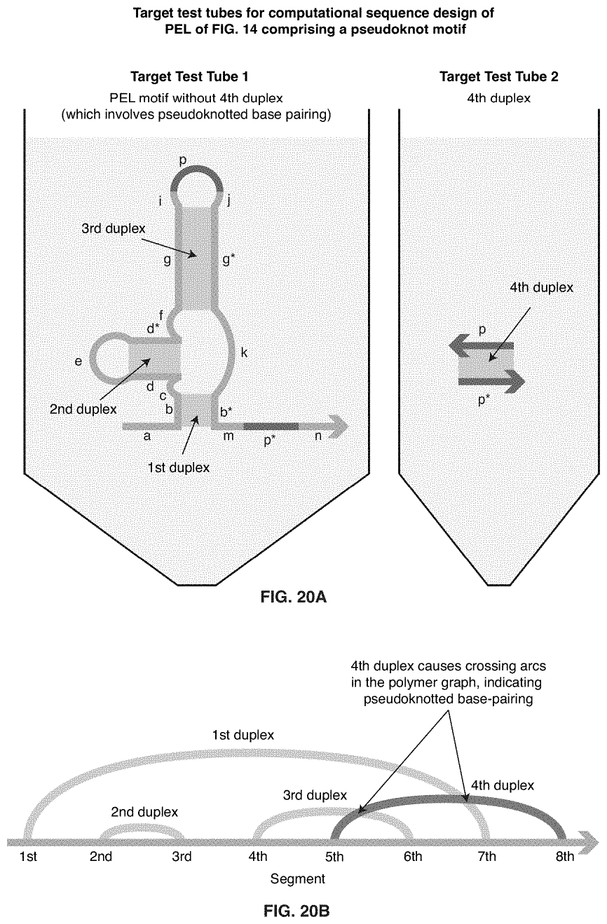

In some embodiments, a PEL motif comprises a pseudoknot motif (see for example the secondary structure schematic of FIG. 18A). In some embodiments, the pseudoknot motif comprises (from 5′ to 3′) a 1^{st} segment, a 2^{nd} segment, a 3^{rd} segment, a 4^{th} segment, a 5^{th} segment, a 6^{th} segment, a 7^{th} segment, an 8^{th} segment, a 9^{th} segment, and a 10^{th} segment. In some embodiments, the 1^{st} segment hybridizes to the 9^{th} segment to form a 1^{st} duplex, the 2^{nd} segment hybridizes to the 8^{th} segment to form a 2^{nd} duplex, the 3^{rd} segment hybridizes to the 4^{th} segment to form a 3^{rd} duplex, the 5^{th} segment hybridizes to the 7^{th} segment to form a 4^{th} duplex, and the 6^{th} segment hybridizes to the 10^{th} segment to form a 5^{th} duplex. These relationships between segments and duplexes are depicted in the secondary structure schematic of FIG. 18A and in the polymer graph schematic of FIG. 18B (in which the segments are depicted 5′ to 3′ along the straight backbone and each duplex is depicted as an arc). In some embodiments, a duplex comprises a set of 1 or more base pairs. In some embodiments, a duplex comprises a set of 2 or more base pairs. In some embodiments, a duplex comprises a set of 2 or more consecutive base pairs. In some embodiments, the 1^{st} segment corresponds to domain “b”, the 2^{nd} segment corresponds to domain “d”, the 3^{rd} segment corresponds to domain “f”, the 4^{th} segment corresponds to domain “p”, the 5^{th} segment corresponds to domain “k”, the 6^{th} segment corresponds to domain “p”, the 7^{th} segment corresponds to domain “k*”, and the 8^{th} segment corresponds to domain “d*”, the 9^{th} segment corresponds to domain “b*”, and the 10^{th} segment corresponds to domain “p*”. In some embodiments, the 1^{st} duplex corresponds to base-pairing between domains “b” and “b*”, the 2^{nd} duplex corresponds to base-pairing between domains “d” and “d*”, the 3^{rd} duplex corresponds to base-pairing between domains “f” and “f*”, the 4^{th} duplex corresponds to base-pairing between domains “k” and “k*”, and the 5^{th} duplex corresponds to base-pairing between domains “p” and “p*”. In some embodiments, there are 0, 1, 2, 3 or more unpaired bases 5′ or 3′ of the any of the numbered segments (these unpaired bases are also known as domains “a”, “c”, “e”, “g”, “h”, “i”, “j”, “m”, “n”, “o”, “q”; see FIG. 18A). In some embodiments, some or all of domains “a”, “c”, “e”, “g”, “h”, “i”, “j”, “m”, “n”, “o”, “q” form intra-domain or inter-domain base pairs. In some embodiments, additional base-pairing and/or tertiary contacts form within the PEL motif. In some embodiments, the PEL motif serves as a mechanical block to prevent nuclease degradation of a nucleic acid comprising the PEL motif.

### PEL Motif (Type 6) Comprising a Pseudoknot Motif

In some embodiments, a PEL motif comprises a pseudoknot motif (see for example the secondary structure schematic of FIG. 19A). In some embodiments, the pseudoknot motif comprises (from 5′ to 3′) a 1^{st} segment, a 2^{nd} segment, a 3^{rd} segment, a 4^{th} segment, a 5^{th} segment, and a 6^{th} segment. In some embodiments, the 1^{st} segment hybridizes to the 5^{th} segment to form a 1^{st} duplex, the 2^{nd} segment hybridizes to the 4^{th} segment to form a 2^{nd} duplex, and the 3^{rd} segment hybridizes to the 6^{th} segment to form a 3^{rd} duplex. These relationships between segments and duplexes are depicted in the secondary structure schematic of FIG. 19A and in the polymer graph schematic of FIG. 19B (in which the segments are depicted 5′ to 3′ along the straight backbone and each duplex is depicted as an arc). In some embodiments, a duplex comprises a set of 1 or more base pairs. In some embodiments, a duplex comprises a set of 2 or more base pairs. In some embodiments, a duplex comprises a set of 2 or more consecutive base pairs. In some embodiments, the 1^{st} segment corresponds to domain “b”, the 2^{nd} segment corresponds to domain “d”, the 3^{rd} segment corresponds to domain “p”, the 4^{th} segment corresponds to domain “d*”, the 5^{th} segment corresponds to domain “b*”, and the 6^{th} segment corresponds to domain “p*”. In some embodiments, the 1^{st} duplex corresponds to base-pairing between domains “b” and “b*”, the 2^{nd} duplex corresponds to base-pairing between domains “d” and “d*”, and the 3^{rd} duplex corresponds to base-pairing between domains “p” and “p*”. In some embodiments, there are 0, 1, 2, 3 or more unpaired bases 5′ or 3′ of the any of the numbered segments (these unpaired bases are also known as domains “a”, “c”, “e”, “f”, “g”, “o”, “q”; see FIG. 19A). In some embodiments, some or all of domains “a”, “c”, “e”, “f”, “g”, “o”, “q” form intra-domain or inter-domain base pairs. In some embodiments, additional base-pairing and/or tertiary contacts form within the PEL motif. In some embodiments, the PEL motif serves as a mechanical block to prevent nuclease degradation of a nucleic acid comprising the PEL motif.

### PEL Motifs (Type 7) Comprising a Pseudoknot Motif

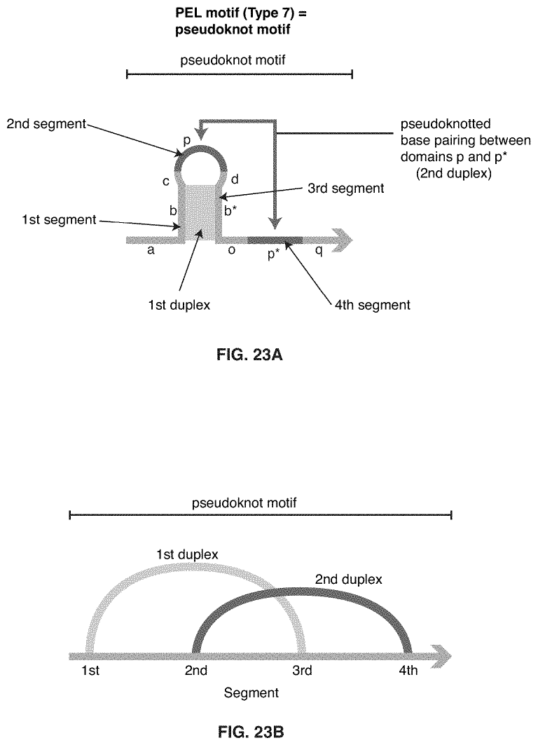

In some embodiments, a PEL motif comprises a pseudoknot motif (see for example the secondary structure schematic of FIG. 23A). In some embodiments, the pseudoknot motif comprises (from 5′ to 3′) a 1^{st} segment, a 2^{nd} segment, a 3^{rd} segment, and a 4^{th} segment. In some embodiments, the 1^{st} segment hybridizes to the 3^{rd} segment to form a 1^{st} duplex and the 2^{nd} segment hybridizes to the 4^{th} segment to form a 2^{nd} duplex. These relationships between segments and duplexes are depicted in the secondary structure schematic of FIG. 23A and in the polymer graph schematic of FIG. 23B (in which the segments are depicted 5′ to 3′ along the straight backbone and each duplex is depicted as an arc). In some embodiments, a duplex comprises a set of 1 or more base pairs. In some embodiments, a duplex comprises a set of 2 or more base pairs. In some embodiments, a duplex comprises a set of 2 or more consecutive base pairs. In some embodiments, the 1^{st} segment corresponds to domain “b”, the 2′ segment corresponds to domain “p”, the 3^{rd} segment corresponds to domain “b*”, and the 4^{th} segment corresponds to domain “p*”. In some embodiments, the 1^{st} duplex corresponds to base-pairing between domains “b” and “b*” and the 2^{nd} duplex corresponds to base-pairing between domains “p” and “p*”. In some embodiments, there are 0, 1, 2, 3 or more unpaired bases 5′ or 3′ of any of the numbered segments (these unpaired bases are also known as domains “a”, “c”, “d”, “o”, and “q”; see FIG. 23A). In some embodiments, some or all of domains “a”, “c”, “d”, “o”, and “q” form intra-domain or inter-domain base pairs. In some embodiments, additional base-pairing and/or tertiary contacts form within the PEL motif. In some embodiments, the PEL motif serves as a mechanical block to prevent nuclease degradation of a nucleic acid comprising the PEL motif.

### PEL Motifs (Type 8) Comprising a Pseudoknot Motif Comprising a Structured Region

In some embodiments, a PEL motif comprises a pseudoknot motif comprising a structured region (see for example the secondary structure schematic of FIG. 24A). In some embodiments, the pseudoknot motif comprises (from 5′ to 3′) a 1^{st} segment, a 2^{nd} segment, a 3^{rd} segment, and a 4^{th} segment. In some embodiments: 1) the 1^{st} segment hybridizes to the 3^{rd} segment to form a structured region comprising a 1^{st} duplex (wherein the structured region additionally comprises none, some, or all of: a) one or more intra-segment base pairs within the 1st segment, b) one or more intra-segment base pairs within the 3^{rd} segment, c) one or more intra-segment base pairs within the 1^{st} segment and/or the 3^{rd} segment interspersed between inter-segment base pairs between the 1^{st} and 3^{rd} segments), and 2) the 2^{nd} segment hybridizes to the 4^{th} segment to form a 2^{nd} duplex. These relationships between segments and duplexes are depicted in the secondary structure schematic of FIG. 24A and in the polymer graph schematic of FIG. 24B (in which the segments are depicted 5′ to 3′ along the straight backbone, the structured region comprising a 1^{st} duplex is depicted as a light gray arc with a dashed boundary, and the 2^{nd} duplex is depicted as a dark gray arc). In some embodiments, a duplex comprises a set of 1 or more base pairs. In some embodiments, a duplex comprises a set of 2 or more base pairs. In some embodiments, a duplex comprises a set of 2 or more consecutive base pairs. In some embodiments, the 1st segment corresponds to domain “b”, the 2^{nd} segment corresponds to domain “p”, the 3^{rd} segment corresponds to domain “e”, and the 4^{th} segment corresponds to domain “p*”. In some embodiments, the 1^{st} duplex corresponds to base-pairing between domains “b” and “e” and the 2^{nd} duplex corresponds to base-pairing between domains “p” and “p*”. In some embodiments, there are 0, 1, 2, 3 or more unpaired bases 5′ or 3′ of any of the numbered segments (these unpaired bases are also known as domains “a”, “c”, “d”, “o”, and “q”; see FIG. 24A). In some embodiments, some or all of domains “a”, “c”, “d”, “o”, and “q” form intra-domain or inter-domain base pairs. In some embodiments, additional base-pairing and/or tertiary contacts form within the PEL motif. In some embodiments, the PEL motif serves as a mechanical block to prevent nuclease degradation of a nucleic acid comprising the PEL motif.

### PEL Motifs (Type 9) Comprising a Pseudoknot Motif Comprising Two Structured Regions

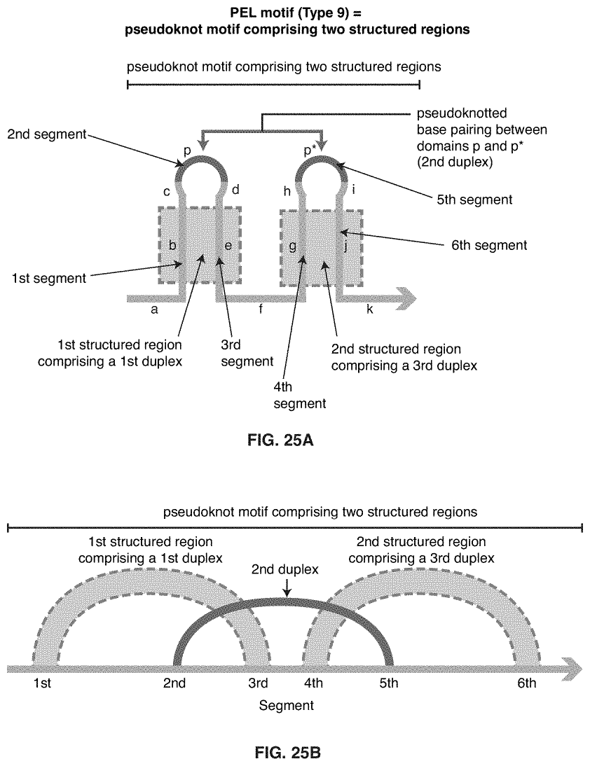

In some embodiments, a PEL motif comprises a pseudoknot motif comprising two structured regions (see for example the secondary structure schematic of FIG. 25A). In some embodiments, the pseudoknot motif comprises (from 5′ to 3′) a 1^{st} segment, a 2^{nd} segment, a 3^{rd} segment, a 4^{th} segment, a 5^{th} segment, and a 6^{th} segment. In some embodiments, the 1^{st} segment hybridizes to the 3^{rd} segment to form a 1^{st} structured region comprising a 1^{st} duplex (wherein the 1^{st} structured region additionally comprises none, some, or all of: a) one or more intra-segment base pairs within the 1^{st} segment, b) one or more intra-segment base pairs within the 3^{rd} segment, c) one or more intra-segment base pairs within the 1^{st} segment and/or the 3^{rd} segment interspersed between inter-segment base pairs between the 1st and 3^{rd} segments), the 2^{nd} segment hybridizes to the 5^{th} segment to form a 2^{nd} duplex, and the 4^{th} segment hybridizes to the 6^{th} segment to form a 2^{nd} structured region comprising a 3^{rd} duplex (wherein the 2^{nd} structured region additionally comprises none, some, or all of: a) one or more intra-segment base pairs within the 4^{th} segment, b) one or more intra-segment base pairs within the 6^{th} segment, c) one or more intra-segment base pairs within the 4^{th} segment and/or the 6^{th} segment interspersed between inter-segment base pairs between the 4^{th} and 6^{th} segments). In some embodiments, the 3^{rd} duplex within the 2^{nd} structured region is formed by hybridization between two sub-segments of the 4^{th} segment or between two sub-segments of the 6^{th} segment (wherein the 2^{nd} structured region additionally comprises none, some, or all of: a) one or more intra-segment base pairs within the 4^{th} segment, b) one or more intra-segment base pairs within the 6^{th} segment, c) one or more intra-segment base pairs within the 4^{th} segment and/or the 6^{th} segment interspersed between inter-segment base pairs between the 4th and 6^{th} segments). The relationships between segments and duplexes are depicted in the secondary structure schematic of FIG. 25A and in the polymer graph schematic of FIG. 25B (in which the segments are depicted 5′ to 3′ along the straight backbone, the 1^{st} structured region comprising a 1^{st} duplex is depicted as a light gray arc with a dashed boundary, the 2^{nd} duplex is depicted as a dark gray arc, and the 2^{nd} structured region comprising a 3^{rd} duplex is depicted as a light gray arc with a dashed boundary). In some embodiments, a duplex comprises a set of 1 or more base pairs. In some embodiments, a duplex comprises a set of 2 or more base pairs. In some embodiments, a duplex comprises a set of 2 or more consecutive base pairs. In some embodiments, the 1^{st} segment corresponds to domain “b”, the 2^{nd} segment corresponds to domain “p”, the 3^{rd} segment corresponds to domain “e”, the 4^{th} segment corresponds to domain “g”, the 5^{th} segment corresponds to domain “p*”, and the 6^{th} segment corresponds to domain “j”. In some embodiments, the 1^{st} duplex corresponds to base-pairing between domains “b” and “e”, the 2^{nd} duplex corresponds to base-pairing between domains “p” and “p*”, and the 3^{rd} duplex corresponds to base-pairing between domains “g” and “j”. In some embodiments, the 3^{rd} duplex corresponds to intra-domain base-pairing within domain “g” or to intra-domain base-pairing within domain “j”. In some embodiments, there are 0, 1, 2, 3 or more unpaired bases 5′ or 3′ of any of the numbered segments (these unpaired bases are also known as domains “a”, “c”, “d”, “f”, “h”, “i”, and “k”; see FIG. 25A). In some embodiments, some or all of domains “a”, “c”, “d”, “f”, “h”, “i”, and “k” form intra-domain or inter-domain base pairs. In some embodiments, additional base-pairing and/or tertiary contacts form within the PEL motif. In some embodiments, the PEL motif serves as a mechanical block to prevent nuclease degradation of a nucleic acid comprising the PEL motif.

### PEL Motifs (Type 10) Comprising a Pseudoknot Motif Comprising a Structured Region

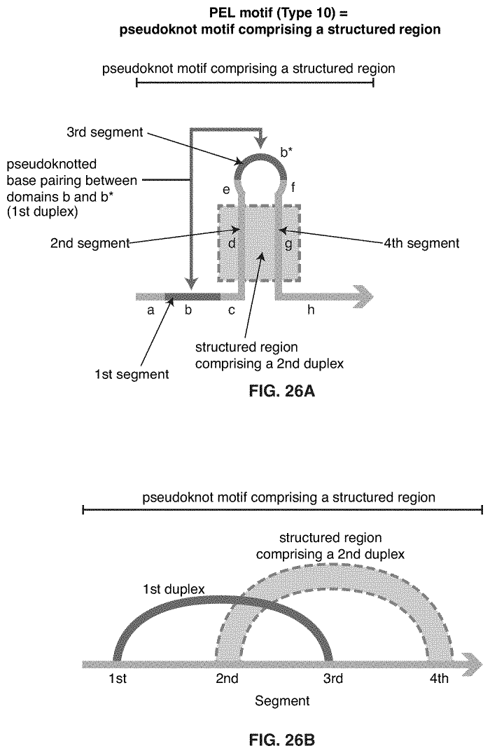

In some embodiments, a PEL motif comprises a pseudoknot motif comprising a structured region (see for example the secondary structure schematic of FIG. 26A). In some embodiments, the pseudoknot motif comprises (from 5′ to 3′) a 1^{st} segment, a 2^{nd} segment, a 3^{rd} segment, and a 4^{th} segment. In some embodiments, the 1^{st} segment hybridizes to the 3^{rd} segment to form a 1^{st} duplex and the 2^{nd} segment hybridizes to the 4^{th} segment to form a structured region comprising a 2^{nd} duplex (wherein the structured region additionally comprises none, some, or all of: a) one or more intra-segment base pairs within the 2^{nd} segment, b) one or more intra-segment base pairs within the 4^{th} segment, c) one or more intra-segment base pairs within the 2^{nd} segment and/or the 4^{th} segment interspersed between inter-segment base pairs between the 2^{nd} and 4^{th} segments). These relationships between segments and duplexes are depicted in the secondary structure schematic of FIG. 26A and in the polymer graph schematic of FIG. 26B (in which the segments are depicted 5′ to 3′ along the straight backbone, the 1^{st} duplex is depicted as a dark gray arc, and the structured region comprising a 2^{nd} duplex is depicted as a light gray arc with a dashed boundary). In some embodiments, a duplex comprises a set of 1 or more base pairs. In some embodiments, a duplex comprises a set of 2 or more base pairs. In some embodiments, a duplex comprises a set of 2 or more consecutive base pairs. In some embodiments, the 1^{st} segment corresponds to domain “b”, the 2^{nd} segment corresponds to domain “d”, the 3^{rd} segment corresponds to domain “b*”, and the 4^{th} segment corresponds to domain “g”. In some embodiments, the 1^{st} duplex corresponds to base-pairing between domains “b” and “b*” and the 2^{nd} duplex corresponds to base-pairing between domains “d” and “g”. In some embodiments, there are 0, 1, 2, 3 or more unpaired bases 5′ or 3′ of any of the numbered segments (these unpaired bases are also known as domains “a”, “c”, “e”, “f”, and “h”; see FIG. 26A). In some embodiments, some or all of domains “a”, “c”, “e”, “f”, and “h” form intra-domain or inter-domain base pairs. In some embodiments, additional base-pairing and/or tertiary contacts form within the PEL motif. In some embodiments, the PEL motif serves as a mechanical block to prevent nuclease degradation of a nucleic acid comprising the PEL motif.

### PEL Motifs (Type 11) Comprising a Structured Region

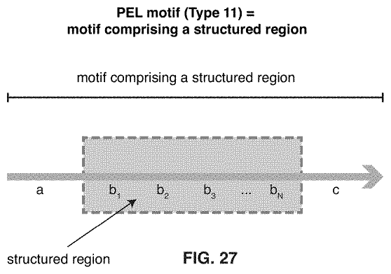

In some embodiments, a PEL motif comprises a structured region (see for example the schematic of FIG. 27). In some embodiments, the structured region comprises (from 5′ to 3′) a sequence domain “a”, sequence domains “b₁”, “b₂”, “b₃”, . . . , “b_{N}”, and a sequence domain “c”. Here, N corresponds to the number of types of “b” domain. In some embodiments, N=2, 3, 4, 5, 6, 7, 8, 9, 10, 11, 12, 13, 14, 15, 16, 17, 18, 19, or 20. In some embodiments, N=10, 20, 30, 40, 50, 60, 70, 80, 90, 100, or any integer number of domains in between any of those values. In some embodiments, N=100, 200, 300, 400, 500, 600, 700, 800, 900, or 1000, or any integer number of domains in between any of those values. In some embodiments, hybridization between two of the domains selected from “b₁”, “b₂”, “b₃”, . . . , “b_{N}” leads to formation of a structured region comprising a 1^{st} duplex. In some embodiments, the structured region further comprises one or more additional duplexes formed via hybridization between pairs of domains selected from “b₁”, “b₂”, “b₃”, . . . , “b_{N}”. In some embodiments, the structured region comprises a pseudoknot motif. In some embodiments, the structured region comprises a hairpin motif. In some embodiments, the hairpin region comprises a pseudoknot motif with a polymer graph with 2, 3, 4, 5, 6, 7, 8, 9, 10, 11, 12, 13, 14, 15, 16, 17, 18, 19, or 20, or more crossing arcs. In some embodiments, the structured region comprises 1 or more pseudoknot motifs and/or 1 or more hairpin motifs and/or 1 or more other motifs each comprising one or more duplexes. In some embodiments, a duplex comprises a set of 1 or more base pairs. In some embodiments, a duplex comprises a set of 2 or more base pairs. In some embodiments, a duplex comprises a set of 2 or more consecutive base pairs. In some embodiments, there are 0, 1, 2, 3 or more unpaired bases 5′ or 3′ of any of the domains “b₁”, “b₂”, “b₃”, . . . , “b_{N}” (including domains “a” and “c”; see FIG. 27). In some embodiments, additional base-pairing and/or tertiary contacts form within the PEL motif. In some embodiments, the PEL motif serves as a mechanical block to inhibit nuclease degradation of a nucleic acid comprising the PEL motif.

### PEL Protection

In some embodiments, a PEL protects from degradation, an RNA strand that serves as an input to a regulatory molecule, complex, or pathway (for example, an RNA trigger that toggles the activity of a conditional guide RNA, or an RNA trigger that toggles the activity of a toehold switch, or an RNA trigger that is recognized as an input by any regulatory molecule, complex, or pathway). In some embodiments, a PEL protects an RNA regulator (for example, a guide RNA, a conditional guide RNA, a toehold switch, a riboregulator, or any other regulator that has a component made of RNA or another nucleic acid or nucleic acid analog). In some embodiments, a PEL protects a molecular logic gate that accepts one or more inputs and produces one or more outputs. In some embodiments, one or more PELs protect one or more inputs accepted by a molecular logic gate. In some embodiments, one or more PELs protect one or more outputs that are produced by a molecular logic gate. In some embodiments, a PEL protects a nucleic acid structure. In some embodiments, a PEL protects an mRNA vaccine. In some embodiments, a PEL protects an mRNA drug. In some embodiments a PEL provides a mechanism for capping and protecting RNAs in a eukaryotic cell. In some embodiments a PEL protects RNAs in a prokaryotic cell. In some embodiments a PEL provides an alternative to vaccinia capping enzyme in the preparation of mRNA vaccines. In some embodiments a PEL provides the same function as a 7-methylguanylate cap.⁶⁷ In some embodiments a PEL increases the efficiency of translation of an RNA in an in vitro translation system (IVTs) such as wheat germ and reticulocyte.⁶⁸ In some embodiments, a PEL protects an mRNA drug. In some embodiments, a PEL protects a DNA, an RNA, or synthetic nucleic acid analog, an mRNA, an rRNA, a tRNA, an miRNA, an siRNA, an antisense RNA, a small RNA, a lncRNA, a non-coding RNA, a coding RNA, an expressed RNA, a synthetic RNA, a synthetic chemically modified nucleic acid, an antisense DNA, an antisense nucleic acid or nucleic acid analog, a chemically modified nucleic acid, or a hybrid molecule that contains two or more types of materials including one or more nucleic acid materials (for example, PNA, XNA, RNA, DNA, 2′OMe-RNA, chemically modified nucleic acids). In some embodiments, a PEL comprises DNA, RNA, 2′OMe-RNA, PNA, XNA, chemically modified nucleic acids, synthesized nucleic acid, expressed nucleic acids, chemical linkers, amino acids, artificial amino acids, or a mixture thereof. In some embodiments, the base-pairing within a PEL motif is Watson-Crick base pairing (for example for RNA: A pairs with U, C pairs with G), or wobble base-pairing (for example, for RNA: G pairs with U). In some embodiments, a PEL motif comprises tertiary contacts including but not limited to base triple, base-phosphate, and/or base-base interactions.⁶

In some embodiments, a PEL is placed 5′ of the sequence domain (or domains) that is to be protected. In some embodiments, a PEL is placed 3′ of the sequence domain (or domains) that is to be protected. In some embodiments, a PEL is placed both 5′ and 3′ of the sequence domain (or domains) that is to be protected. In some embodiments, a molecule intersperses PELs between domains that are to be protected. For example, a long RNA could alternate (5′ to 3′) between PELs and domains to be protected. In some embodiments, a self-cleaving ribozyme within a long RNA cleaves the RNA to expose a PEL 5′ or 3′ of a sequence to be protected. In some embodiments, a PEL is placed at the 5′ end of a nucleic acid strand. In some embodiments, a PEL is placed at the 3′ end of a nucleic acid strand. In some embodiments, PELs are placed at both the 5′ and 3′ ends of a strand. In some embodiments, PELs are placed at one or more locations within a strand.

In some applications, it is desirable for a PEL motif to be as short as possible (as few nucleotides as possible) so as to minimize base-pairing and/or steric interactions between the PEL and the nucleic acid sequence that is to be protected by the PEL, as well as to minimize interactions between the PEL and other molecules that are intended to interact with the protected sequence proximal to the PEL. In some embodiments, it is beneficial to use a PEL motif that is significantly shorter than naturally occurring viral xrRNA motifs. For example, in some embodiments, it is beneficial to use a PEL motif consisting of a single pseudoknot motif without an accompanying hairpin motif (for example FIG. 4A) in contrast to a viral xrRNA that consists of a pseudoknot motif and a hairpin motif, or a viral xrRNA that consists of a first pseudoknot motif, a first hairpin motif, a second pseudoknot motif, a second hairpin motif, possible additional motifs, and intervening linker sequences. In some embodiments, it is beneficial to use a PEL motif consisting of a single pseudoknot motif and a single hairpin motif in contrast to a viral xrRNA that consists of a first pseudoknot motif, a first hairpin motif, a second pseudoknot motif, a second hairpin motif, and possible additional motifs. In some embodiments, it is desirable to use a PEL motif that comprises a pseudoknot. In some embodiments, it is desirable to use a PEL motif that does not comprise a pseudoknot. For example, a PEL motif could be intentionally designed to be as small as possible such that it is too short to form a pseudoknot.

In some embodiments, PELs reduce degradation of a nucleic acid by 10%, 20%, 50%, 60%, 70%, 80%, 90%, 95%, 99%, 99.5%, 99.9%, or more. In some embodiments, PELs protect RNA. In some embodiments, PELs protect DNA. In some embodiments, PELs protect chemically synthesized nucleic acids. In some embodiments, PELs protect chemically modified nucleic acids or nucleic acid analogs. In some embodiments, PELs protect expressed nucleic acids. In some embodiments, PELs protect molecules containing one or more nucleic acid domains of the same or different nucleic acid materials, of as well as possibly other domains that are not nucleic acids (for example, chemical linkers not capable of base-pairing, amino acids, non-natural amino acids, etc). In some embodiments, PELs reduce degradation of nucleic acids on the bench top, in a test tube, in permeablized samples, in fixed samples, in living organisms, in lysates, in prokaryotes, in eukaryotic cells, in tissues, in organs, in embryos, in adult organisms, in viruses, in mammals, in humans, in plants, in ecosystems, in space, and/or in the biosphere. In some embodiments, PELs protect nucleic acids that enhance the performance of nucleic acid synthetic biology. In some embodiments, PELs enable a conditional response that is 2-fold, 5-fold, 10-fold, 20-fold, 50-fold, 100-fold, 200-fold, 500-fold, or 1000-fold, or more. In some embodiments, PELs increase fold-change of a regulatory response by a factor of 2, 5, 10, 20, 50, 100, 200, 500, 1000, or more. In some embodiments, PELs enable a fractional dynamic range of 10%, 20%, 30%, 40%, 50%, 60%, 70%, 80%, 90%, 95%, 99%, 99.5%, 99.9%, or more. In some embodiments, PELs increase fractional dynamic range by 2-fold, 5-fold, 10-fold, 20-fold, 50-fold, 100-fold, or more. In some embodiments, PELs increase the longevity of nucleic acids by 2-fold, 5-fold, 10-fold, 20-fold, 50-fold, 100-fold, 200-fold, 500-fold, 1000-fold, 2000-fold, 5000-fold, 10,000-fold, or more. In some embodiments, PELs reduce degradation of an RNA trigger that serves as an input to a regulatory pathway. In some embodiments, PELs reduce degradation of an RNA that serves as a substrate for mediating a chemical reaction. In some embodiments, PELs reduces degradation of an RNA therapeutic within the cell.

In some embodiments, the linker region between any pair of pseudoknot pseudoknot motifs, hairpin motifs, and/or structured regions can be shortened or lengthened so that it contains a total of 0, 1, 2, 5, 10, 20, 50, 100, 200, 500, or 1000 nt, or any number of nucleotides intermediate to these values. In some embodiments, the PEL sequences derived from components of viral xrRNAs can be adjusted via rational design or directed evolution. In some embodiments, the sequence of a PEL represents a combination of subsequences from multiple viral xrRNAs. In some embodiments, any of the pseudoknot motifs, hairpin motifs, and/or structured regions used in different types of PEL motifs (for example, Types 1-11) can be combined in any order. In some embodiments, any PEL motif derived from any virus can be combined with a PEL motif derived from any other virus. In some embodiments, PEL motifs derived from one or more viruses can be combined with rationally designed PEL motifs and/or sequences. In some embodiments, non-naturally-occurring PEL motifs are designed rationally and/or engineered using directed evolution.

### Computational Sequence Design of PEL Motifs

In some embodiments, the sequence of a PEL motif is rationally designed using a computer algorithm, manually designed by a human or by multiple humans, or designed via machine learning. In some embodiments, the PEL sequence is rationally designed using NUPACK^{69,70} or another computational sequence design tool. In some embodiments, sequence design is formulated as a multistate optimization problem using multiple target test tubes. In some embodiments, each target test tube contains a set of desired on-target complexes (each with a target secondary structure and target concentration) and a set of undesired off-target complexes (each with vanishing target concentration).⁷⁰ In some embodiments, a PEL is designed using two target test tubes. For example, FIG. 20A depicts target test tubes for the computational sequence design of the PEL (Type 1) of FIG. 14A comprising a pseudoknot motif. In FIG. 20A, the first target test tube contains an on-target complex comprising a single strand that is the full length of the PEL, with a target secondary structure comprising the duplexes in the PEL motif except for any pseudoknotted duplexes (that is, except for any duplex that leads to crossing arcs in the polymer graph). In this example, the 4^{th} duplex is excluded from the first target test tube because it leads to crossing arcs in the polymer graph of the PEL motif (FIG. 20B). In some embodiments, the off-target complexes in the first target test tube are dimers formed from base-pairing between two PEL motifs. In some embodiments, there are no off-target complexes in the first target test tube. In FIG. 20A, the second target test tube includes as on-target complexes any duplexes that were excluded from the first target test tube. In this example, the second target test tube contains the 4^{th} duplex as an on-target complex. In some embodiments, the off-target complexes in the second target test tube are the individual segments intended to base-pair to form the duplex (for example, sequence domains “p” and “p*”). In some embodiments, there are no off-target complexes in the second target test tube. In some embodiments, sequence design is performed subject to complementarity constraints inherent to the PEL motif (for example in FIG. 20A, domain “b” complementary to domain “b*”, etc). In some embodiments, biological sequence constraints or other sequence constraints are imposed. In some embodiments, sequences are optimized by reducing the ensemble defect quantifying the average fraction of incorrectly paired nucleotides over the multi-tube ensemble.⁷⁰ In some embodiments, defect weights are applied within the ensemble defect to prioritize design effort.⁷⁰ In some embodiments, optimization of the ensemble defect implements both a positive design paradigm, explicitly design for on-pathway elementary steps, and a negative design paradigm, explicitly design against off-pathway crosstalk.⁷⁰ In some embodiments, a PEL is designed using one, two, or more target test tubes. In some embodiments, two or more PELs are designed simultaneously using one, two, or more target test tubes. In some embodiments, the target concentration for the on-target complexes is the same or different for each target test tube. In some embodiments, the target concentration is 1 μM, or 1 nM, or 1 pM, or 1 fM, or 1 aM, or 1 zM, or above or below or between any of those concentrations. In some embodiments, PEL sequences are obtained using directed evolution starting from a PEL sequence that is rationally designed or from a PEL sequence that is derived from a viral xrRNA. In some embodiments, the structure of the PEL motif is rationally designed prior to rational design of the PEL sequence. In some embodiments, rational design of the PEL motif involves some or all of: 1) specification of the number of segments, 2) specification of the length of each segment, 3) specification of the complementarity relationships between segments.

Although the foregoing invention has been described in terms of certain preferred embodiments, other embodiments will be apparent to those of ordinary skill in the art. Additionally, other combinations, omissions, substitutions, and modifications will be apparent to the skilled artisan, in view of the disclosure herein. Accordingly, the present invention is not intended to be limited by the recitation of the preferred embodiments, but is instead to be defined by reference to the appended claims. All references cited herein are incorporated by reference in their entirety.

### ARRANGEMENTS

In addition to the foregoing, some embodiments provide the following arrangements:

Arrangement 1: A protective element (PEL) within a synthesized or expressed RNA molecule that reduces degradation of at least one sequence element 5′ and/or 3′ of the PEL, wherein the at least one sequence element that experiences reduced degradation is known as a protected sequence.

Arrangement 2: A protective element (PEL) within a nucleic acid, wherein the PEL comprises a structured region comprising one or more duplexes, and wherein the structured region reduces degradation of a protected sequence 5′ and/or 3′ of the PEL.

Arrangement 3: The PEL of Arrangement 1 or 2, wherein the PEL comprises a PEL motif comprising a pseudoknot motif: the pseudoknot motif comprising (from 5′ to 3′) a 1^{st} segment, a 2^{nd} segment, a 3^{rd} segment, a 4^{th} segment, a 5^{th} segment, a 6^{th} segment, a 7^{th} segment, and an 8^{th} segment, wherein the 1^{st} segment hybridizes to the 7^{th} segment to form a 1^{st} duplex, the 2^{nd} segment hybridizes to the 3^{rd} segment to form a 2^{nd} duplex, the 4^{th} segment hybridizes to the 6^{th} segment to form a 3^{rd} duplex, and the 5^{th} segment hybridizes to the 8^{th} segment to form a 4^{th} duplex.

Arrangement 4: The PEL motif of Arrangement 3 wherein an additional duplex forms between bases 5′ of the 1^{st} segment and bases 3′ of the 6^{th} segment and 5′ of the 7^{th} segment.

Arrangement 5: The PEL of Arrangement 1 or 2, wherein the PEL comprises a PEL motif comprising (from 5′ to 3′) a pseudoknot motif and a hairpin motif: a. the pseudoknot motif comprising (from 5′ to 3′) a 1^{st} segment, a 2^{nd} segment, a 3^{rd} segment, a 4^{th} segment, a 5^{th} segment, a 6^{th} segment, a 7^{th} segment, and an 8^{th} segment, wherein the 1^{st} segment hybridizes to the 7^{th} segment to form a 1^{st} duplex, the 2^{nd} segment hybridizes to the 3^{rd} segment to form a 2^{nd} duplex, the 4^{th} segment hybridizes to the 6^{th} segment to form a 3^{rd} duplex, the 5^{th} segment hybridizes to the 8^{th} segment to form a 4^{th} duplex; and b. the hairpin motif comprising (from 5′ to 3′) a 9^{th} segment and a 10^{th} segment, wherein the 9^{th} segment hybridizes to the 10^{th} segment to form a 5^{th} duplex.

Arrangement 6: The PEL of Arrangement 5 wherein an additional duplex forms between bases 5′ of the 1^{st} segment and bases that are 3′ of the 6^{th} segment and 5′ of the 7^{th} segment.

Arrangement 7: The PEL of Arrangement 1 or 2, wherein the PEL comprises a PEL motif comprising (from 5′ to 3′) a first pseudoknot motif and a second pseudoknot motif: a. the first pseudoknot motif comprising (from 5′ to 3′) a 1^{st} segment, a 2^{nd} segment, a 3^{rd} segment, a 4^{th} segment, a 5^{th} segment, a 6^{th} segment, a 7^{th} segment, and an 8^{th} segment, wherein the 1^{st} segment hybridizes to the 7^{th} segment to form a 1^{st} duplex, the 2^{nd} segment hybridizes to the 3^{rd} segment to form a 2^{nd} duplex, the 4^{th} segment hybridizes to the 6^{th} segment to form a 3^{rd} duplex, and the 5^{th} segment hybridizes to the 8^{th} segment to form a 4^{th} duplex; and b. the second pseudoknot motif comprising (from 5′ to 3′) a 9^{th} segment, a 10^{th} segment, an 11^{th} segment, a 12^{th} segment, a 13^{th} segment, a 14^{th} segment, a 15^{th} segment, and a 16^{th} segment, wherein the 9^{th} segment hybridizes to the 15^{th} segment to form a 5^{th} duplex, the 10^{th} segment hybridizes to the 11^{th} segment to form a 6^{th} duplex, the 12^{th} segment hybridizes to the 14^{th} segment to form a 7^{th} duplex, and the 13^{th} segment hybridizes to the 16^{th} segment to form an 8^{th} duplex.

Arrangement 8: The PEL motif of Arrangement 7 wherein an additional duplex forms between bases 5′ of the 1^{st} segment and bases 3′ of the 6^{th} segment and 5′ of the 7^{th} segment.

Arrangement 9: The PEL motif of Arrangement 7 wherein an additional duplex forms between bases 5′ of the 9th segment and bases 3′ of the 14^{th} segment and 5′ of the 15^{th} segment.

Arrangement 10: The PEL of Arrangement 1 or 2, wherein the PEL comprises a PEL motif comprising (from 5′ to 3′) a first pseudoknot motif, a first hairpin motif, a second pseudoknot motif, and a second hairpin motif: a. the first pseudoknot motif comprising (from 5′ to 3′) a 1^{st} segment, a 2^{nd} segment, a 3^{rd} segment, a 4^{th} segment, a 5^{th} segment, a 6^{th} segment, a 7^{th} segment, and an 8^{th} segment, wherein the 1^{st} segment hybridizes to the 7^{th} segment to form a 1^{st} duplex, the 2^{nd} segment hybridizes to the 3^{rd} segment to form a 2^{nd} duplex, the 4^{th} segment hybridizes to the 6^{th} segment to form a 3^{rd} duplex, and the 5^{th} segment hybridizes to the 8^{th} segment to form a 4^{th} duplex; b. the first hairpin motif comprising (from 5′ to 3′) a 9^{th} segment and a 10^{th} segment, wherein the 9^{th} segment hybridizes to the 10^{th} segment to form a 5^{th} duplex; c. the second pseudoknot motif comprising (from 5′ to 3′) an 11^{th} segment, a 12^{th} segment, a 13^{th} segment, a 14^{th} segment, a 15^{th} segment, a 16^{th} segment, a 17^{th} segment, and an 18^{th} segment, wherein the 11^{th} segment hybridizes to the 17^{th} segment to form a 6^{th} duplex, the 12^{th} segment hybridizes to the 13^{th} segment to form a 7^{th} duplex, the 14^{th} segment hybridizes to the 16^{th} segment to form an 8^{th} duplex, and the 15^{th} segment hybridizes to the 18^{th} segment to form a 9^{th} duplex; and d. the second hairpin motif comprising (from 5′ to 3′) a 19^{th} segment and a 20^{th} segment, wherein the 19^{th} segment hybridizes to the 20^{th} segment to form a 10^{th} duplex.

Arrangement 11: The PEL motif of Arrangement 10 wherein an additional duplex forms between bases 5′ of the 1^{st} segment and bases 3′ of the 6^{th} segment and 5′ of the 7^{th} segment.

Arrangement 12: The PEL motif of Arrangement 10 wherein an additional duplex forms between bases 5′ of the 11^{th} segment and bases 3′ of the 16^{th} segment and 5′ of the 17^{th} segment.

Arrangement 13: The PEL of Arrangement 1 or 2, wherein the PEL comprises a PEL motif comprising a pseudoknot motif: the pseudoknot motif comprising (from 5′ to 3′) a 1^{st} segment, a 2^{nd} segment, a 3^{rd} segment, a 4^{th} segment, a 5^{th} segment, a 6^{th} segment, a 7^{th} segment, an 8^{th} segment, a 9^{th} segment, and a 10^{th} segment, wherein the 1^{st} segment hybridizes to the 9^{th} segment to form a 1^{st} duplex, the 2^{nd} segment hybridizes to the 8^{th} segment to form a 2^{nd} duplex, the 3^{rd} segment hybridizes to the 4^{th} segment to form a 3^{rd} duplex, the 5^{th} segment hybridizes to the 7^{th} segment to form a 4^{th} duplex, and the 6^{th} segment hybridizes to the 10^{th} segment to form a 5^{th} duplex.

Arrangement 14: The PEL of Arrangement 1 or 2, wherein the PEL comprises a PEL motif comprising a pseudoknot motif: the pseudoknot motif comprising (from 5′ to 3′) a 1^{st} segment, a 2^{nd} segment, a 3^{rd} segment, a 4^{th} segment, a 5^{th} segment, and a 6^{th} segment, wherein the 1^{st} segment hybridizes to the 5^{th} segment to form a 1^{st} duplex, the 2′ segment hybridizes to the 4^{th} segment to form a 2^{nd} duplex, and the 3^{rd} segment hybridizes to the 6^{th} segment to form a 3^{rd} duplex.

Arrangement 15: The PEL of Arrangement 1 or 2, wherein the PEL comprises a PEL motif comprising a pseudoknot motif: the pseudoknot motif comprising (from 5′ to 3′) a 1^{st} segment, a 2nd segment, a 3^{rd} segment, and a 4^{th} segment, wherein the 1^{st} segment hybridizes to the 3^{rd} segment to form a 1^{st} duplex and the 2^{nd} segment hybridizes to the 4^{th} segment to form a 2^{nd} duplex.

Arrangement 16: The PEL of Arrangement 1 or 2, wherein the PEL comprises a PEL motif comprising a pseudoknot motif: the pseudoknot motif comprising (from 5′ to 3′) a 1^{st} segment, a 2^{nd} segment, a 3^{rd} segment, and a 4^{th} segment, wherein the 1^{st} segment hybridizes to the 3^{rd} segment to form a structured region comprising a 1^{st} duplex and the 2^{nd} segment hybridizes to the 4^{th} segment to form a 2^{nd} duplex.

Arrangement 17: The PEL of Arrangement 16 wherein the structured region additionally comprises one or more of: a. one or more intra-segment base pairs within the 1^{st} segment; b. one or more intra-segment base pairs within the 3^{rd} segment; and c. one or more intra-segment base pairs within the 1^{st} segment and/or the 3^{rd} segment interspersed between inter-segment base pairs between the 1^{st} and 3^{rd} segments.

Arrangement 18: The PEL of Arrangement 1 or 2, wherein the PEL comprises a PEL motif comprising a pseudoknot motif: the pseudoknot motif comprising (from 5′ to 3′) a 1^{st} segment, a 2^{nd} segment, a 3^{rd} segment, a 4^{th} segment, a 5^{th} segment, and a 6^{th} segment, wherein the 1^{st} segment hybridizes to the 3^{rd} segment to form a 1^{st} structured region comprising a 1st duplex, the 2^{nd} segment hybridizes to the 5^{th} segment to form a 2^{nd} duplex, and the 4^{th} segment hybridizes to the 6^{th} segment to form a 2^{nd} structured region comprising a 3^{rd} duplex.

Arrangement 19: The PEL of Arrangement 1 or 2, wherein the PEL comprises a PEL motif comprising a pseudoknot motif: the pseudoknot motif comprising (from 5′ to 3′) a 1^{st} segment, a 2^{nd} segment, a 3^{rd} segment, a 4^{th} segment, a 5^{th} segment, and a 6^{th} segment, wherein the 1^{st} segment hybridizes to the 3^{rd} segment to form a 1^{st} structured region comprising a 1^{st} duplex, the 2^{nd} segment hybridizes to the 5^{th} segment to form a 2^{nd} duplex, and a 3rd duplex is formed within a 2^{nd} structured region by hybridization between two sub-segments of the 4^{th} segment or between two sub-segments of the 6^{th} segment.

Arrangement 20: The PEL of Arrangements 18 or 19 wherein the 1st structured region additionally comprises one or more of: a. one or more intra-segment base pairs within the 1^{st} segment; b. one or more intra-segment base pairs within the 3^{rd} segment; and c. one or more intra-segment base pairs within the 1st segment and/or the 3^{rd} segment interspersed between inter-segment base pairs between the 1^{st} and 3^{rd} segments; and/or the 2^{nd} structured region additionally comprises one or more of: a. one or more intra-segment base pairs within the 4^{th} segment; b. one or more intra-segment base pairs within the 6^{th} segment; and c. one or more intra-segment base pairs within the 4^{th} segment and/or the 6^{th} segment interspersed between inter-segment base pairs between the 4^{th} and 6^{th} segments.

Arrangement 21: The PEL of Arrangement 1 or 2, wherein the PEL comprises a PEL motif comprising a pseudoknot motif: the pseudoknot motif comprising (from 5′ to 3′) a 1st segment, a 2^{nd} segment, a 3^{rd} segment, and a 4^{th} segment, wherein the 1st segment hybridizes to the 3^{rd} segment to form a 1^{st} duplex and the 2^{nd} segment hybridizes to the 4^{th} segment to form a structured region comprising a 2^{nd} duplex.

Arrangement 22: The PEL of Arrangement 21 wherein the structured region additionally comprises one or more of: a. one or more intra-segment base pairs within the 2^{nd} segment; b. one or more intra-segment base pairs within the 4^{th} segment; and c. one or more intra-segment base pairs within the 2^{nd} segment and/or the 4^{th} segment interspersed between inter-segment base pairs between the 2^{nd} and 4^{th} segments.

Arrangement 23: The PEL of Arrangement 1 or 2, wherein the PEL comprises a PEL motif comprising a structured region, the structured region comprising a first duplex, wherein the structured region serves as a mechanical block to inhibit nuclease degradation of the protected sequence.

Arrangement 24: The PEL of Arrangement 23, wherein the structured region comprises one, two, three, or more additional duplexes.

Arrangement 25: The PEL of Arrangement 23, wherein the structured region comprises a pseudoknot.

Arrangement 26: The PEL of any one of the preceding Arrangements wherein additional base-pairing and/or tertiary contacts form within the PEL motif, including but not limited to base pairs, base triples, base-phosphate interactions, and base-base interactions.

Arrangement 27: The PEL of any one of the preceding Arrangements wherein consecutive motifs within a PEL (from 5′ to 3′) are connected by a linker comprising zero, one, or more nucleotides or alternatively comprising a material not capable of base-pairing.

Arrangement 28: The PEL of any one of the preceding Arrangements wherein the PEL reduces degradation of an exogenous RNA molecule in a eukaryotic cell.

Arrangement 29: The PEL of any one of the preceding Arrangements wherein the protected sequence is an mRNA vaccine.

Arrangement 30: The PEL of any one of the preceding Arrangements wherein the protected sequence is an RNA drug.

Arrangement 31: The PEL of any one of the preceding Arrangements wherein the protected sequence mediates the function of an endogenous biological pathway.

Arrangement 32: The PEL of any one of the preceding Arrangements wherein the protected sequence functions as a regulator.

Arrangement 33: The PEL of any one of the preceding Arrangements wherein the protected sequence functions as a logic gate that accepts one or more inputs and conditionally produces one or more outputs.

Arrangement 34: The PEL of any one of the preceding Arrangements wherein the protected sequence serves as a structural element in an assembly of multiple structural elements.

Arrangement 35: The PEL of any one of the preceding Arrangements wherein the protected sequence serves as a substrate for mediating the interaction of other molecules.

Arrangement 36: The PEL of any one of the preceding Arrangements wherein the protected sequence mediates the function of the CRISPR/Cas pathway.

Arrangement 37: The PEL of Arrangement 36 wherein the protected sequence is a trigger sequence that activates a previously inactive conditional guide RNA (cgRNA), allowing the cgRNA to direct Cas-mediated induction, silencing, editing, binding, epigenome editing, chromatin interaction mapping and regulation, or imaging of a target gene within a eukaryotic cell or prokaryote.

Arrangement 38: The PEL of Arrangement 36 wherein the protected sequence is a trigger sequence that inactivates a previously active conditional guide RNA, stopping the cgRNA from further directing Cas-mediated induction, silencing, or editing, binding, epigenome editing, chromatin interaction mapping and regulation, or imaging of a target gene within a eukaryotic cell or prokaryote.

Arrangement 39: The PEL of any one of the preceding Arrangements wherein the protected sequence is translated by an in vitro translation system.

Arrangement 40: The PEL of any one of the preceding Arrangements wherein the PEL is used to replace a 7-methylguanylate cap on an RNA.

Arrangement 41: The PEL of one of the preceding Arrangements wherein at least some or all of the PEL sequence is derived from a component of a viral xrRNA.

Arrangement 42: The PEL of any one of the preceding Arrangements wherein none of the PEL sequence is derived from a component of a viral xrRNA.

Arrangement 43: The PEL of any one of the preceding Arrangements wherein the PEL comprises RNA, DNA, 2′OMe-RNA, chemically modified nucleic acids, synthetic nucleic acid analogs, PNA, XNA, any other material capable of base-pairing, one or more chemical linkers not capable of base-pairing, or any combination thereof.

Arrangement 44: The PEL of any one of the preceding Arrangements wherein the protected sequence comprises RNA, DNA, 2′OMe-RNA, chemically modified nucleic acids, synthetic nucleic acid analogs, PNA, XNA, any other material capable of base-pairing, one or more chemical linkers not capable of base-pairing, or any combination thereof.

Arrangement 45: The PEL of any one of the preceding Arrangements wherein the PEL comprises a PEL motif comprising a duplex that comprises 1, 2, 3, 4, 5, 6, 7, 8, 9, 10, 11, 12, 13, 14, 15, 16, 17, 18, 19, or 20 consecutive base pairs between two segments.

Arrangement 46: The PEL of any one of the preceding Arrangements wherein the PEL comprises a PEL motif comprising a duplex that comprises 1, 2, 3, 4, 5, 6, 7, 8, 9, 10, 11, 12, 13, 14, 15, 16, 17, 18, 19, or 20 base pairs between two segments with 1 or more mismatches (corresponding to unpaired bases) interspersed at one or more locations between the base pairs.

Arrangement 47: A method of reducing degradation of a nucleic acid in a sample, comprising: providing a synthesized or expressed RNA molecule which includes a protective element (PEL) according to any one of Arrangements 1 and 3 to 46; and combining the RNA molecule including the PEL with a sample comprising at least one other molecule; wherein the PEL reduces degradation of at least one sequence element 5′ and/or 3′ of the PEL and the at least one sequence element that experiences reduced degradation is known as a protected sequence.

Arrangement 48: A method of reducing degradation of a nucleic acid in a sample, comprising: providing a protective element (PEL) according to any one of Arrangements 2 to 46; and combining the nucleic acid containing the PEL with a sample comprising at least one other molecule; wherein the PEL comprises a structured region that reduces nuclease-mediated degradation of a protected sequence 5′ and/or 3′ of the PEL.

### EXAMPLES

### Example—Protective Element (PEL) Sequences and Structures

FIG. 4A illustrates a PEL motif (Type 1) comprising a pseudoknot motif; see FIG. 4G for example PEL sequences derived from components of viral xrRNAs.¹ FIG. 4B illustrates a PEL motif (Type 2) comprising a pseudoknot motif and a hairpin motif; see FIG. 4H for example PEL sequences derived from components of viral xrRNAs.^{1,4} FIG. 4C illustrates a PEL motif (Type 3) comprising a first pseudoknot motif and a second pseudoknot motif; see FIG. 4I for example PEL sequences derived from components of viral xrRNAs.¹ FIG. 4D illustrates a PEL motif (Type 4) comprising a first pseudoknot motif, a first hairpin motif, a second pseudoknot motif, and a second hairpin motif; see FIG. 4J for example PEL sequences derived from components of viral xrRNAs.¹ FIG. 4E illustrates a PEL motif (Type 5) comprising a pseudoknot motif; see FIG. 4K for example PEL sequences derived from components of viral xrRNAs.⁶ FIG. 4F illustrates a PEL motif (Type 6) comprising a pseudoknot motif; see FIG. 4L for example PEL sequences derived from components of viral xrRNAs.^{7,71} FIG. 21A illustrates a PEL motif (Type 7) comprising a pseudoknot motif; see FIG. 22A for example PEL sequences that were computationally designer⁷². FIG. 21B illustrates a PEL motif (Type 8) comprising a pseudoknot motif comprising a structured region; see FIGS. 4G, 4K, 4L, and 22A for example PEL sequences derived from components of viral xrRNAs.^{1,6,7,71,72} FIG. 21C illustrates a PEL motif (Type 9) comprising a pseudoknot motif comprising two structured regions; see FIGS. 4H-4J for example PEL sequences derived from components of viral xrRNAs.^{1,4} FIG. 21D illustrates a PEL motif (Type 10) comprising a pseudoknot motif comprising a structured region; see FIG. 22B for example PEL sequences that combine biological sequence information with rational design.^{6,7,71-73} FIG. 21E illustrates a PEL motif (Type 11) comprising a structured motif; see FIGS. 4G-4L and 22A-22B for example PEL sequences derived from components of viral xrRNAs^{1,4,6,7,71} or computationally designed.⁷² In FIGS. 4G-4L and 22A-22B, PEL sequences are listed 5′ to 3′. Nucleotides within pseudoknot and/or hairpin motifs are upper case. Nucleotides in an optional linker region between pseudoknot and/or hairpin motifs are lower case.

In some embodiments, the linker region between any pair of pseudoknot motifs, hairpin motifs, and/or structured regions can be shortened or lengthened so that it contains a total of 0, 1, 2, 5, 10, 20, 50, 100, 200, 500, or 1000 nt, or any number of nucleotides intermediate to these values. In some embodiments, the PEL sequences derived from components of viral xrRNAs can be adjusted via rational design or directed evolution. In some embodiments, the sequence of a PEL represents a combination of subsequences from multiple viral xrRNAs. In some embodiments, any of the pseudoknot motifs, hairpin motifs, and/or structured regions used in different types of PEL motifs (for example, Types 1-11) can be combined in any order. In some embodiments, any PEL motif derived from any virus can be combined with a PEL motif derived from any other virus. In some embodiments, PEL motifs derived from one or more viruses can be combined with rationally designed PEL motifs and/or sequences. In some embodiments, non-naturally-occurring PEL motifs are designed rationally and/or engineered using directed evolution.

### Example—Logic, Function, Structure, and Interactions of a Standard Guide RNA (gRNA)

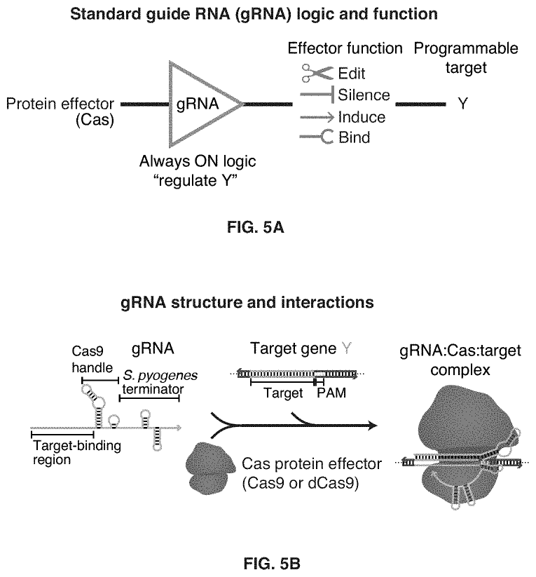

FIG. 5A depicts the logic and function of a standard guide RNA (gRNA). A standard gRNA is ON, unconditionally directing the activity of a protein effector to a target Y; different Cas variants implement different functions including editing, silencing, inducing, binding, epigenome editing, chromatin interaction mapping and regulation, or imaging. FIG. 5B depicts structure and interactions of a standard gRNA. From 5′ to 3′, a standard gRNA comprises: a target-binding region, a Cas handle recognized by the protein effector, and a terminator region.

### Example—Logic and Function of a Conditional Guide RNA (cgRNA)

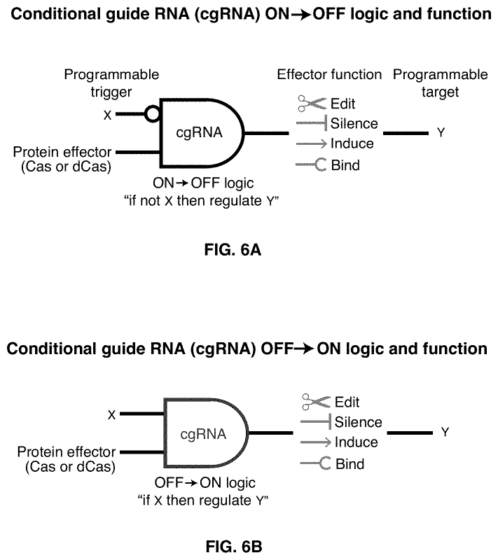

FIG. 6 depicts the logic and function of a conditional guide RNA (cgRNA). A cgRNA changes conformation in response to a programmable trigger X to conditionally direct the activity of a protein effector to a programmable target Y. Top: ON→OFF logic with a constitutively active cgRNA that is conditionally inactivated by X. Bottom: OFF→ON logic with a constitutively inactive cgRNA that is conditionally activated by X.

### Example—Enhancing Nucleic Acid Synthetic Biology Performance Using PELs in Human Cells

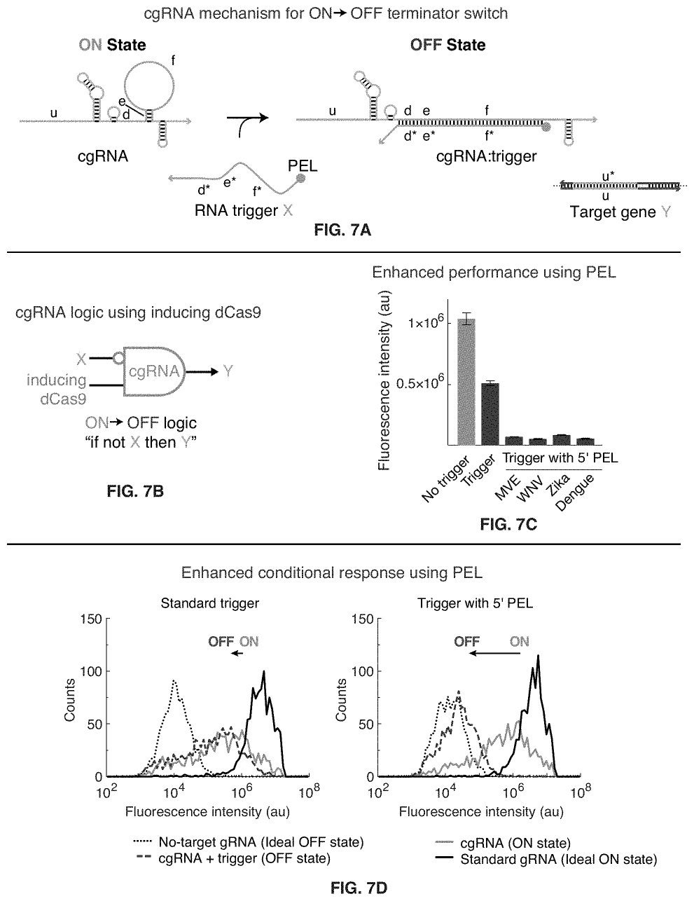

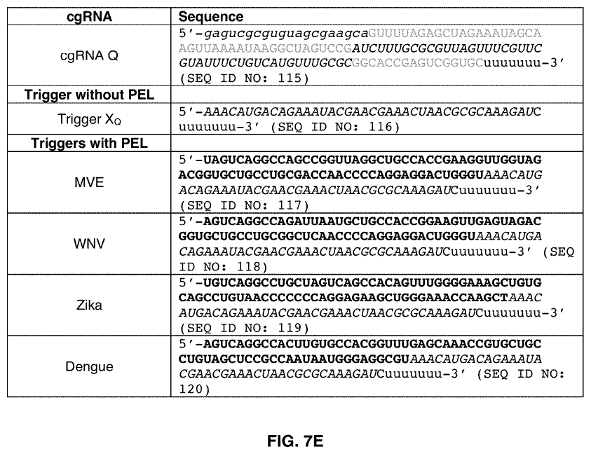

FIG. 7 depicts an example of enhancing nucleic acid synthetic biology performance using PELs in HEK 293 T cells. FIG. 7A depicts the mechanism for an allosteric ON→OFF terminator switch cgRNA: the constitutively active cgRNA is inactivated by hybridization of RNA trigger X. Rational design of cgRNA terminator region (domains “d-e-f”: 6 nt linker, 4 nt stem, 30 nt loop) and complementary trigger region (domains “f*-e*-d*”). FIG. 7B depicts the conditional logic for a terminator switch cgRNA used in conjunction with inducing dCas9: “if not X then Y” (induce target gene Y if trigger X is not detected). FIG. 7C demonstrates achieving a cleaner OFF state and a stronger ON→OFF conditional response using triggers protected with a PEL. This performance benefit is illustrated using PEL motifs derived from different viruses: Murray Valley encephalitis (MVE), West Nile virus (WNV), Zika, and Dengue 4. Raw fluorescence depicting ON→OFF conditional response to a standard trigger or a trigger protected with PEL in HEK 293 T cells. All samples include the terminator switch cgRNA Q. The no-trigger control uses a random pool of triggers to provide a sequence-generic approximation of the metabolic load of trigger expression. All of the remaining samples use terminator switch trigger X_{Q} with the noted PEL motif appended 5′ of the trigger. Bar graphs depict mean±estimated standard error of the mean calculated based on the mean single-cell fluorescence over 487-3906 cells for each of N=3 replicate wells. FIG. 7D displays single-cell fluorescence intensities via flow cytometry, demonstrating the improvement in OFF state and ON→OFF conditional response using an RNA trigger protected by a PEL motif representing a fragment of a Dengue 4 xrRNA (right panel, Dengue) compared to an RNA trigger without PEL protection (left panel). FIG. 7E depicts the sequence of cgRNA Q and the sequences of trigger X_{Q} with or without a 5′ PEL. PEL motifs are derived from different viruses (MVE, WNV, Zika, and Dengue 4). Nucleotides that are lower case italic are constrained by the target binding site on the reporter plasmid. Nucleotides shaded gray are constrained by dCas9. Nucleotides that are upper case italic are designed. The plain “C” nucleotide is a cloning artifact. Lower case plain nucleotides are constrained by the hU6 terminator sequence⁷⁴. Bold nucleotides are constrained by a PEL sequence from: Murray Valley encephalitis (MVE, NC_000943.1)^{1,8}, West Nile virus (WNV, NC_001563.2)¹, Zika (NC_012532.1)¹, or Dengue (Dengue 4, NC_002640.1)¹.

### Example—Enhancing Nucleic Acid Synthetic Biology Performance for Multiple Orthogonal Regulators Using PELs in Human Cells

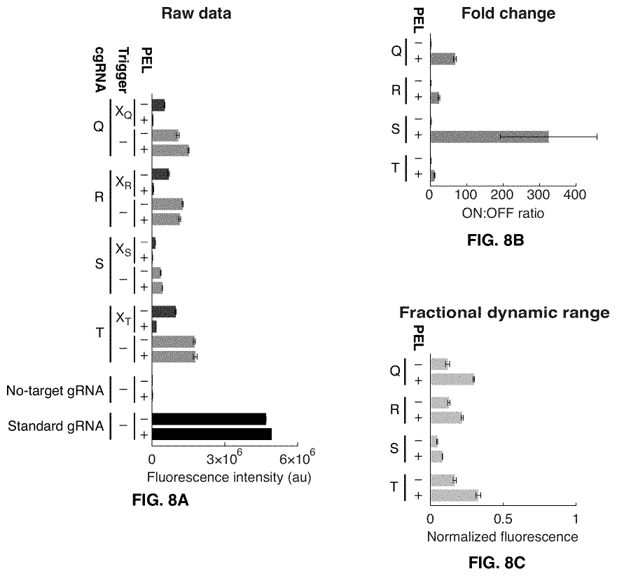

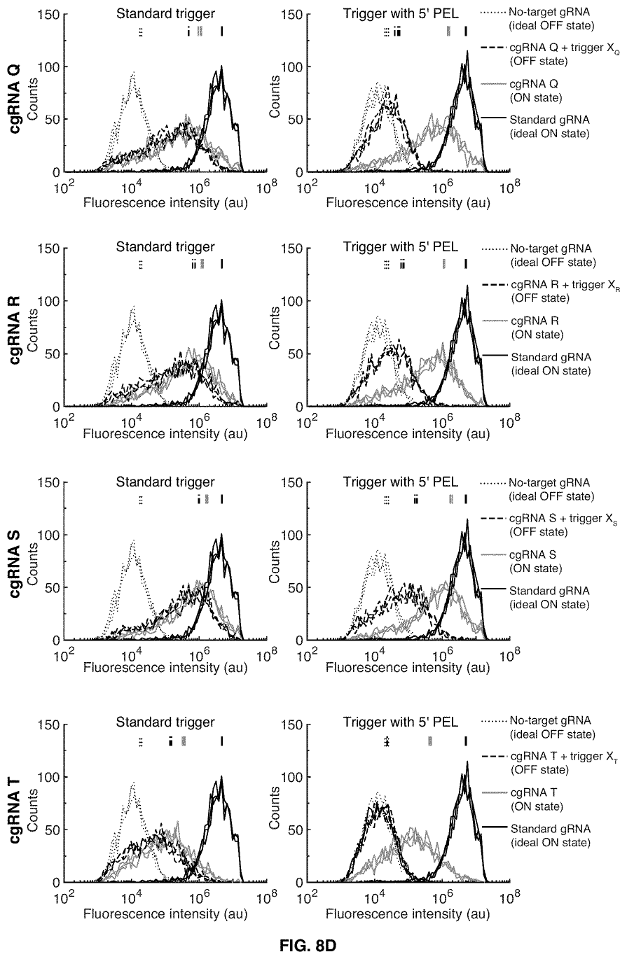

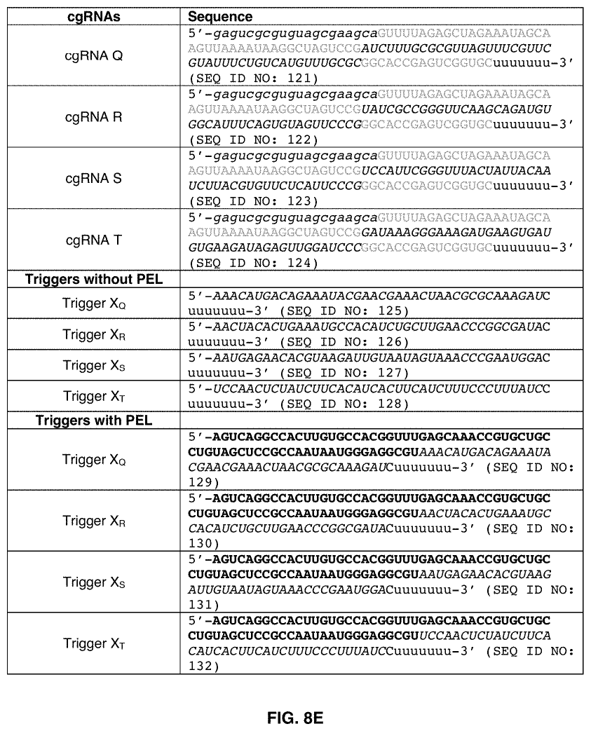

FIG. 8 depicts an example of enhancing nucleic acid synthetic biology using PELs in HEK 293T cells in the context of multiple orthogonal RNA regulators. FIG. 8A demonstrates the substantial improvement in conditional response for a library of four terminator switch cgRNAs (Q, R, S, T; ON→OFF logic) using cognate triggers (X_{Q}, X_{R}, X_{S}, X_{T}) protected by a 5′ PEL (derived from Dengue 4, NC_002640.1)¹ compared to cognate triggers lacking the PEL. The cleaner OFF state using triggers with a 5′ PEL leads to increases in fold change (FIG. 8B) and fractional dynamic range (FIG. 8C). In FIG. 8, expression of RNA trigger X (±PEL+40 nt unstructured+hU6 terminator) toggles the cgRNA from ON→OFF, leading to a decrease in fluorescence. Transfection of plasmids expressing inducing dCas9-VPR, Phi-YFP target gene Y, and either: standard gRNA+no-trigger control (ideal ON state), cgRNA+no-trigger control (ON state), cgRNA+RNA trigger (X_{Q} for cgRNA Q, X_{R} for cgRNA R, X_{S} for cgRNA S, X_{T} for cgRNA T; OFF state)), no-target gRNA that lacks target-binding region+no-trigger control (ideal OFF state). FIG. 8 illustrates programmable conditional regulation using 4 orthogonal cgRNAs (Q, R, S, T). In FIG. 8A, raw fluorescence depicts ON→OFF conditional response to cognate trigger. In FIG. 8B, fold change=ON/OFF. In FIG. 8C, fractional dynamic range=(ON−OFF)/(ideal ON−ideal OFF). Bar graphs depict mean±estimated standard error of the mean (with uncertainty propagation) calculated based on the mean single-cell fluorescence over 1067-4358 cells for each of N=3 replicate wells. Fold-change: maximize the ON→OFF or OFF→ON conditional response ratio with/without the cognate RNA trigger (higher is better). Fractional dynamic range: maximize the difference between conditional ON and OFF states as a fraction of the unconditional regulatory dynamic range of CRISPR/Cas using standard gRNAs (higher is better).

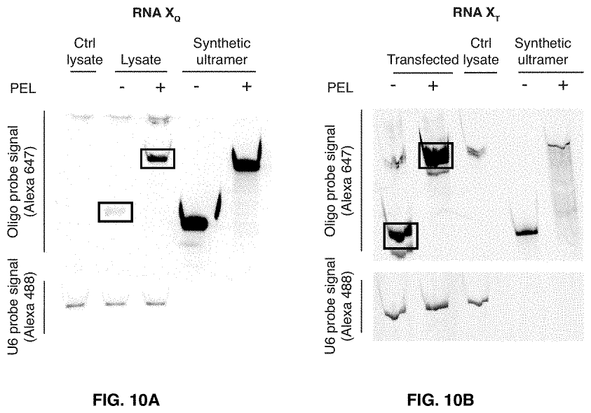

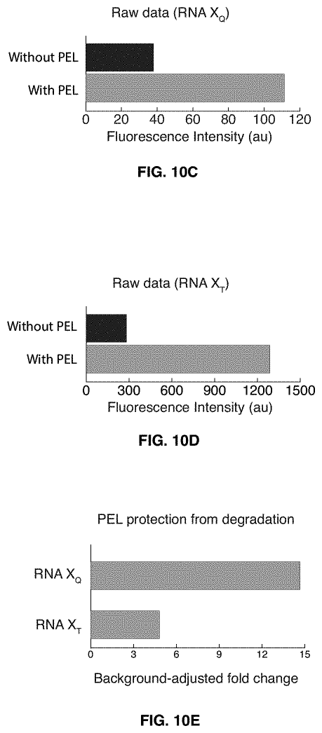

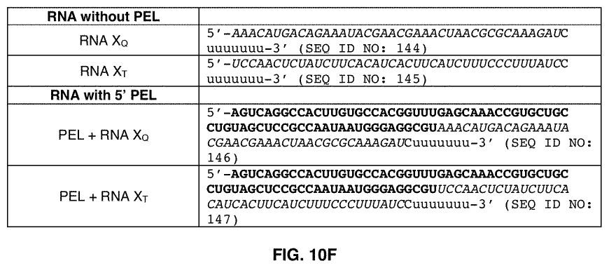

FIG. 8D displays single-cell fluorescence intensities for flow cytometry replicates. Traces of the same line type correspond to N=3 replicate wells transfected on the same day and assayed via flow cytometry 24 h post transfection (M=1000 cells from the high-transfection gate per well). The mean for each replicate is displayed as a vertical line. FIG. 8E depicts the sequences of cgRNAs Q, R, S, T, and the sequences of triggers X_{Q}, X_{R}, X_{S}, X_{T} with or without a 5′ PEL. Nucleotides that are lower case italic are constrained by the target binding site on the reporter plasmid. Nucleotides shaded gray are constrained by dCas9. Nucleotides that are upper case italic are designed. The plain “C” nucleotide is a cloning artifact. Lower case plain nucleotides are constrained by the hU6 terminator sequence⁷⁴. Bold nucleotides represent a PEL sequence constrained by a portion of an xrRNA sequence derived from Dengue (Dengue 4, NC_002640.1)¹. The northern blots of FIG. 10 verify that the 5′ PEL significantly protects triggers X_{Q} and X_{T} from degradation relative to triggers without a PEL.

The orthogonal cgRNA/trigger pairs for the studies of FIG. 8 were designed using NUPACK^{69,70}. A cgRNA expression plasmid and a trigger expression plasmid were co-transfected with a plasmid expressing an inducing dCas9-VPR fusion⁷⁵ and a reporter plasmid containing a gRNA binding site upstream of a minimal CMV promoter for Phi-YFP expression.^{76,77} The four plasmids were transiently transfected into HEK 293T cells with Lipofectamine 2000 and grown for 24 h, with end-point fluorescence measured via flow cytometry. Data analysis was performed on cells expressing high levels of both cgRNA and trigger fluorescent protein transfection controls.

### Example—Enhancing Nucleic Acid Synthetic Biology Performance Using Different PEL Variants in Human Cells

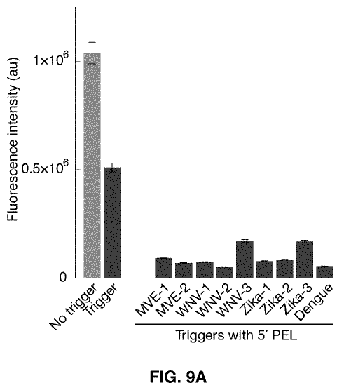

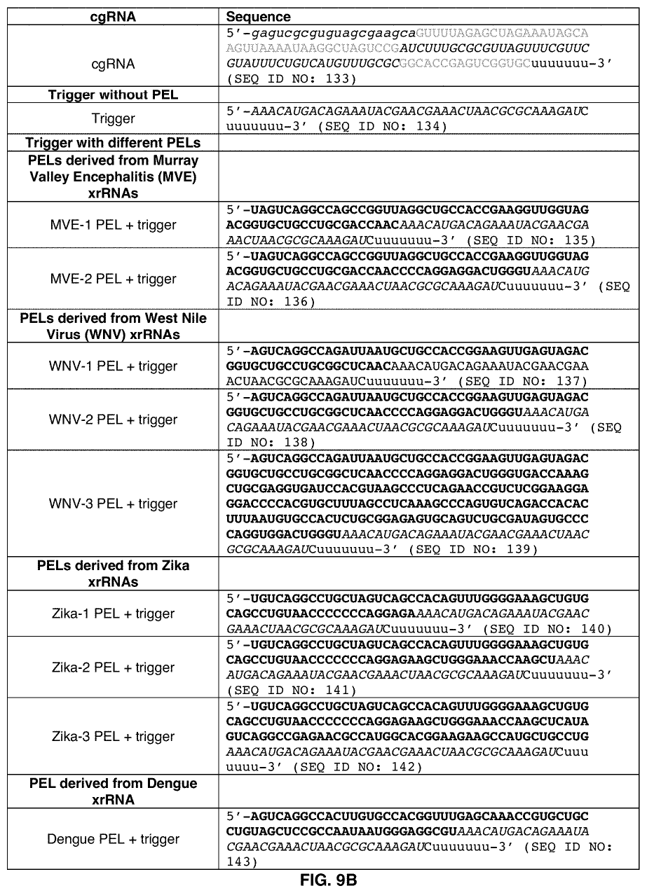

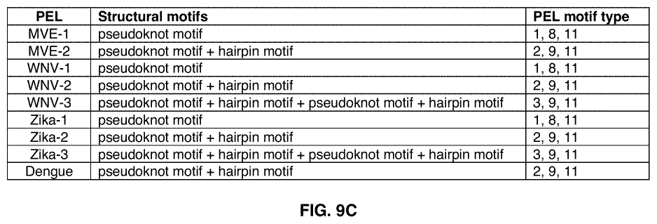

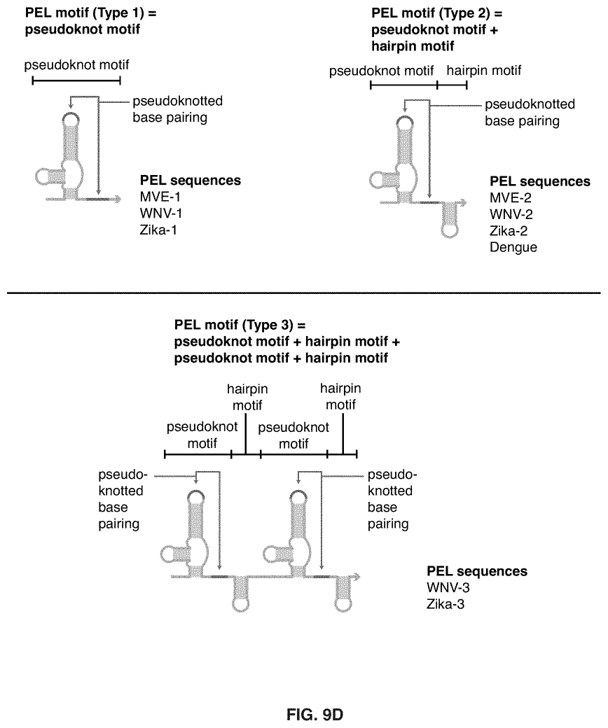

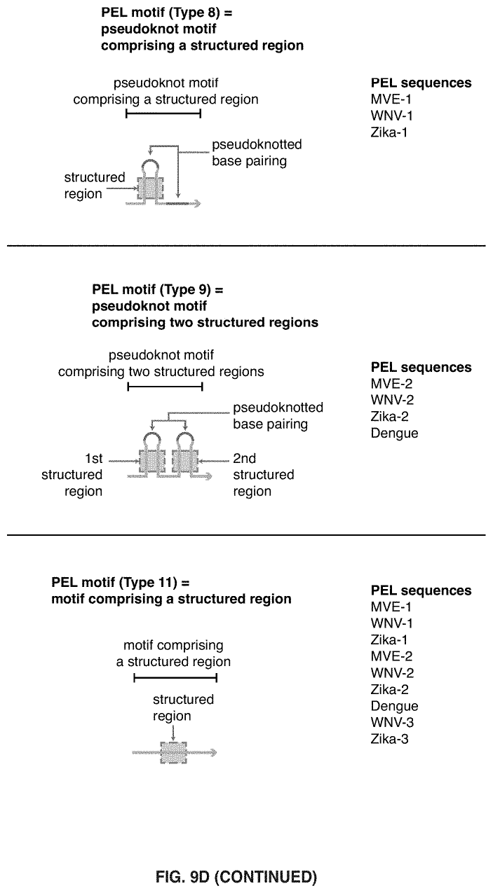

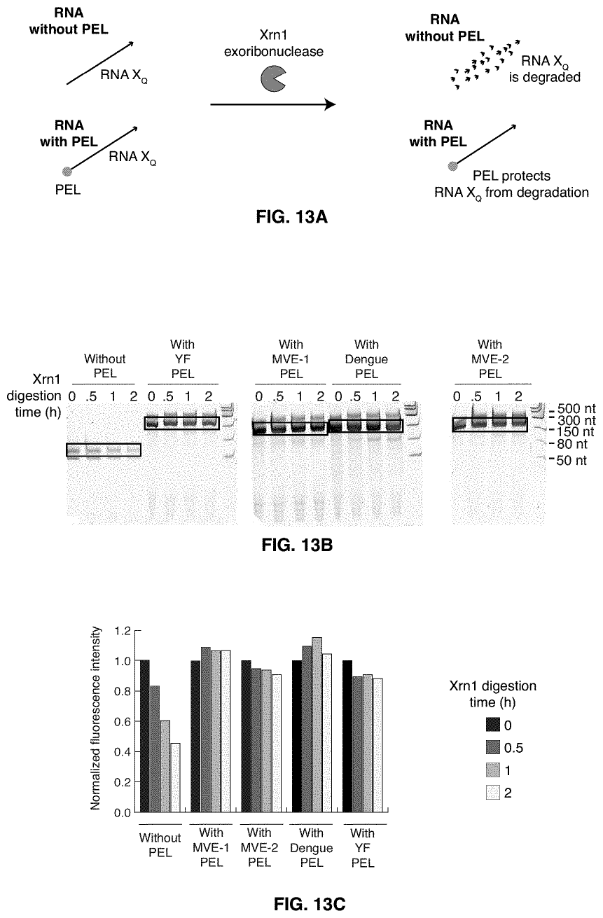

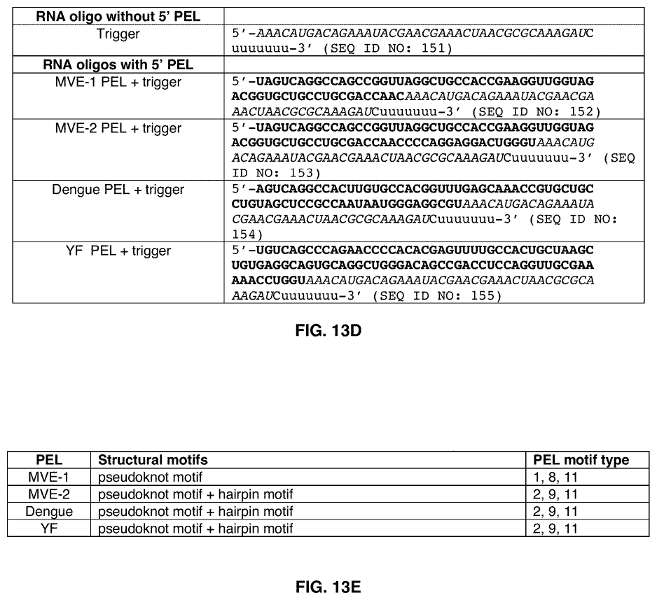

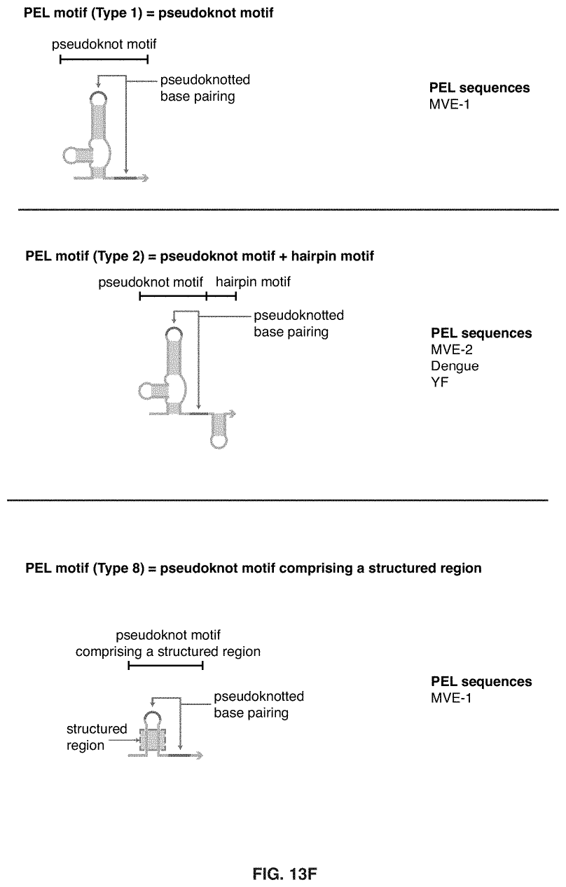

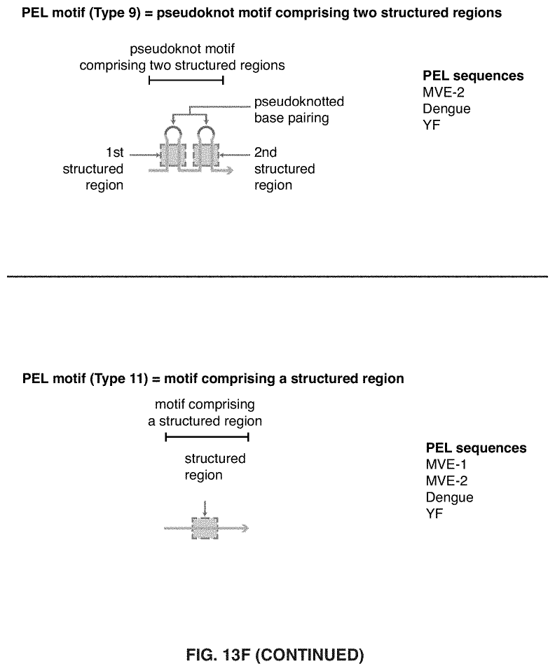

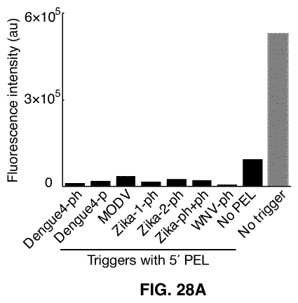

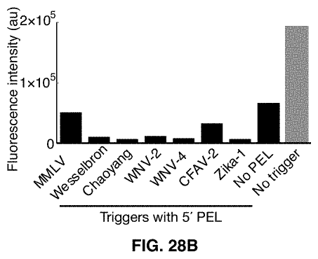

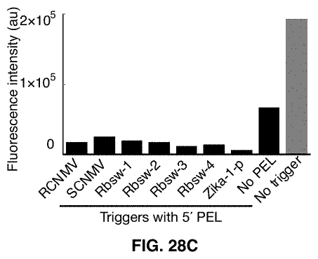

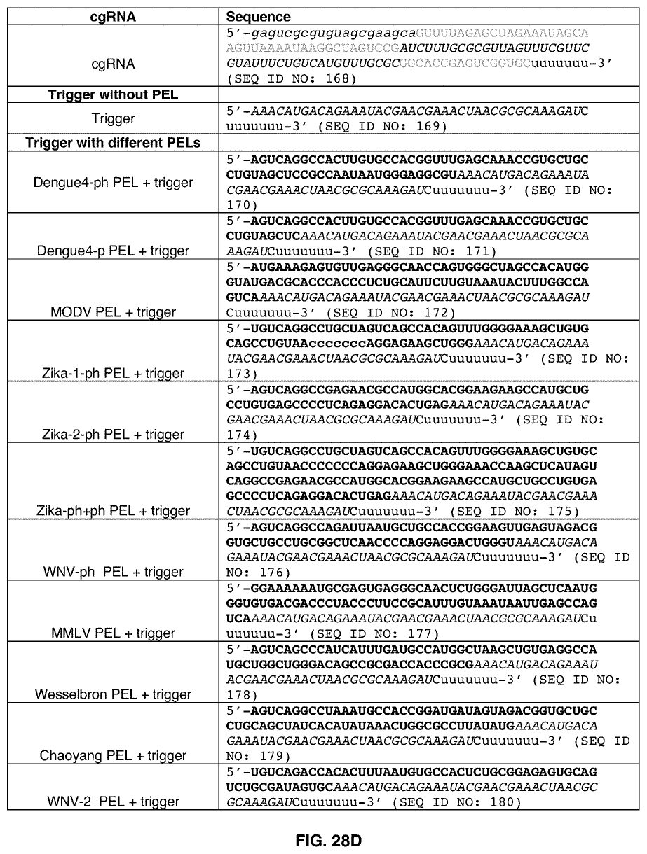

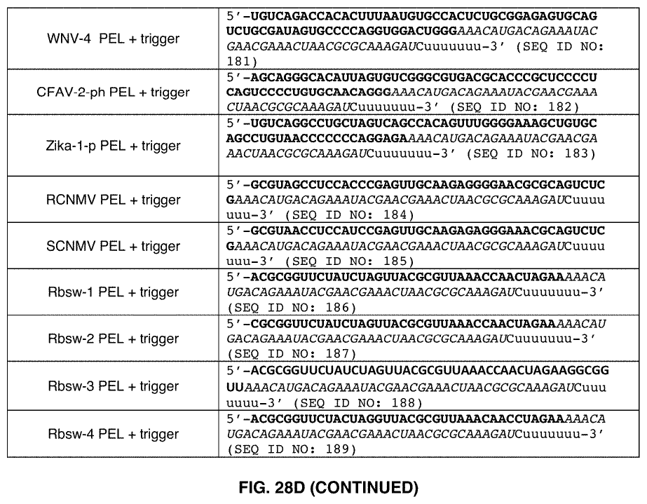

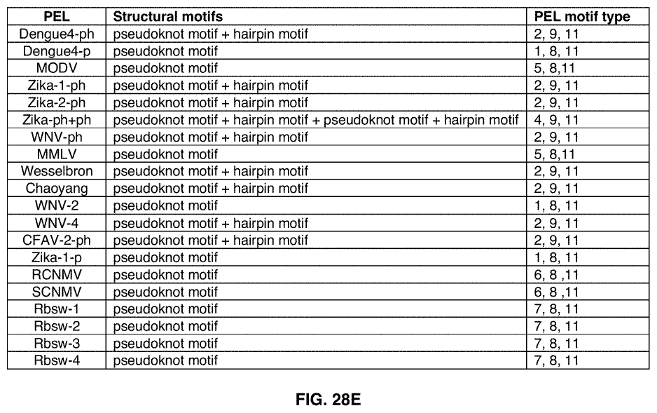

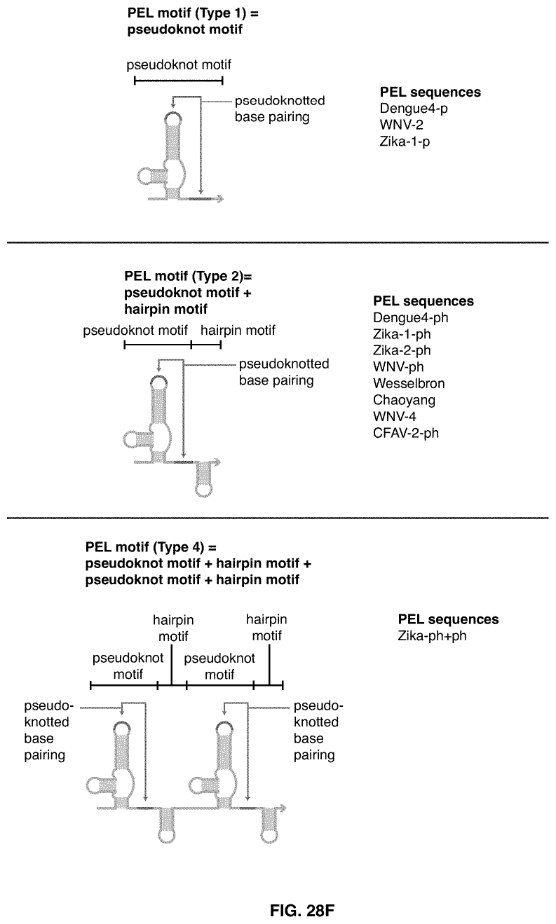

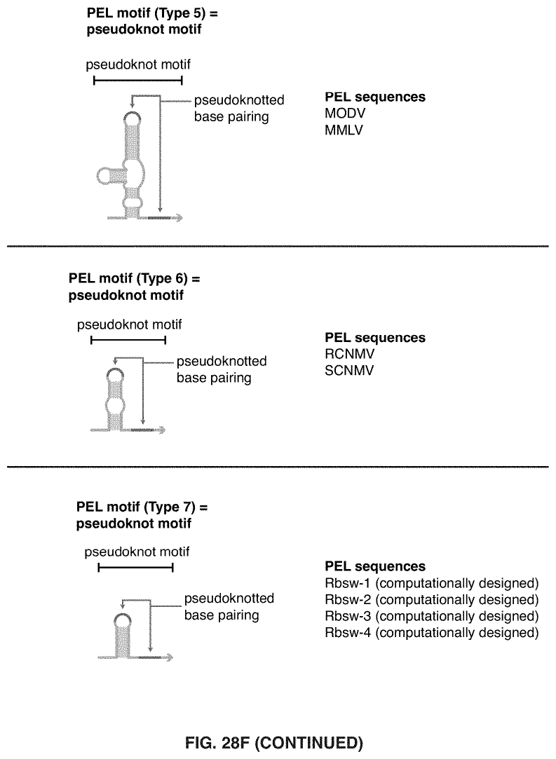

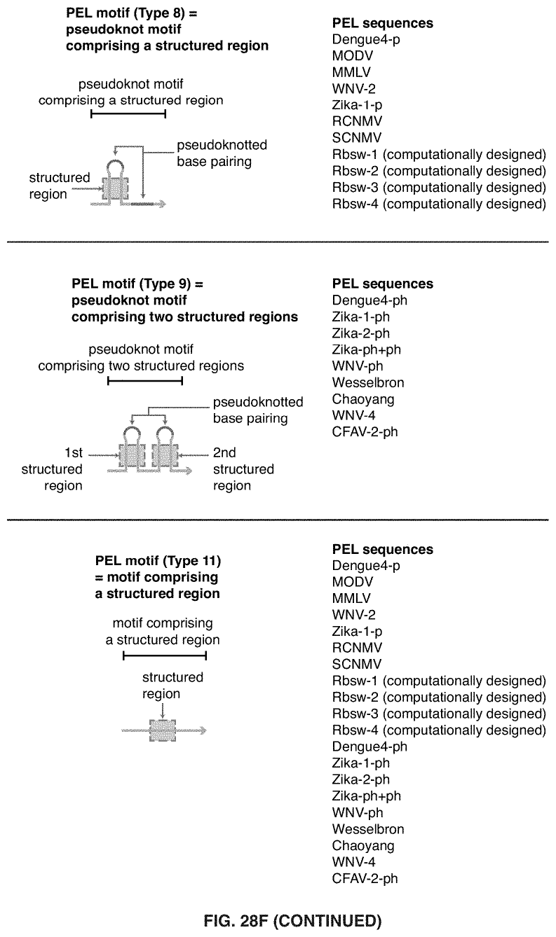

FIGS. 9 and 28 depict an example of enhancing nucleic acid synthetic biology using PELs in HEK293T cells in the context of multiple PEL motifs derived from different viruses. FIGS. 9A and 28A-28C demonstrate the substantial improvement in the OFF state for a terminator switch cgRNA using a trigger protected by any of 9 different PEL motifs derived from 4 different viruses (FIG. 9A), 7 different PEL motifs derived from 4 different viruses (FIG. 28A), 7 different PEL motifs derived from 6 different viruses (FIG. 28B), 3 different PEL motifs derived from 3 different viruses and 4 different PEL motifs that were designed computationally (FIG. 28C). Expression of RNA trigger X (±PEL+40 nt unstructured+hU6 terminator) toggles the cgRNA from ON→OFF, leading to a decrease in fluorescence. Transfection of plasmids expressing inducing dCas9-VPR, Phi-YFP target gene Y, and either: cgRNA+no-trigger control (ON state), cgRNA+RNA trigger X (OFF state). The “No trigger” control (ON state) uses a random pool of triggers to provide a sequence-generic approximation of the metabolic load of trigger expression. The “Trigger” sample (OFF state) uses a trigger without a PEL. All of the remaining samples use a trigger with PEL motif appended 5′ of the trigger (enhanced OFF state). Bar graphs in FIG. 9A depict mean±estimated standard error of the mean calculated based on the mean single-cell fluorescence over 487-3906 cells for each of N=3 replicate wells. Bar graphs in FIG. 28A-28C depict the mean single-cell fluorescence over 1042-9193 cells for one well. FIG. 9B and FIG. 28D depict the sequences of the cgRNA and the trigger with or without a 5′ PEL. PEL motifs are derived from different viruses or are computationally designed. Nucleotides that are lower case italic are constrained by the target binding site on the reporter plasmid. Nucleotides shaded gray are constrained by dCas9. Nucleotides that are upper case italic are designed cgRNA or trigger sequence. The plain “C” nucleotide is a cloning artifact. Lower case plain nucleotides are constrained by the hU6 terminator sequence.⁷⁴ Bold nucleotides in FIG. 9B represent a PEL sequence constrained by a portion of an xrRNA sequence from: Murray Valley encephalitis (MVE, NC_000943.0,^{1,8} West Nile virus (WNV, NC_001563.2),¹ Zika (NC_012532.1),¹ or Dengue (Dengue 4, NC_002640.1).¹ Bold nucleotides in FIG. 28D represent either: 1) a PEL sequence constrained by a portion of an xrRNA sequence from: Dengue4 (Dengue, NC_002640.1),¹ Modoc virus (MODV),⁶ Zika (NC_012532.1),¹ West Nile virus (WNV, NC_001563.2),¹ Montana myotis leukoencephalitis virus (MMLV),⁶ Wesselbron,¹ Chaoyang,¹ Cell fusing agent virus (CFAV),⁶ Red clover necrotic mosaic virus (RCNMV),⁷¹ Sweet clover necrotic mosaic virus (SCNMV),⁷¹ or 2): a computationally designed riboswitch (Rbsw) sequence⁷². FIGS. 9C and 28E describe the pseudoknot and hairpin motifs used in each PEL variant. FIGS. 9D and 28F depict the secondary structure of the pseudoknot and hairpin motifs used in each PEL motif.¹ Gray shading denotes duplex regions; darker domains base pair to each other to form pseudoknotted base pairs. An arrowhead denotes the 3′ end of each strand. The digestion studies of FIGS. 13 and 29 examine a selection of these PELs to confirm that they protect trigger X_{Q} from digestion by exoribonuclease Xrn1.

### Example—Using PELs to Protect Exogenous RNAs from Degradation in Human Cells

FIG. 10 illustrates that a PEL protects RNAs from degradation in living cells. HCR northern blots⁷⁸ (FIGS. 10A and 10B) are used to examine the abundance of two RNAs (RNA X_{Q} and RNA X_{T}) in lysate from HEK 293T cells. By transfecting plasmids into the HEK 293 T cells, the oligos are expressed either with or without a 5′ PEL motif derived from Dengue (Dengue 4, NC_002640.1).¹ Band identities for targets detected in the lysate are verified using synthetic oligos synthesized with and without the 5′ PEL. U6 small non-coding RNA is used as a loading control to verify that the cellular expression levels are comparable between lanes. For a given northern blot, both oligo targets (with or without PEL) are detected with the same pair of HCR probes. Detection of the target oligo colocalizes the two probes in the probe pair, colocalizing a full HCR initiator that initiates HCR signal amplification via polymerization of a tethered HCR amplification polymer assembled from HCR hairpins each carrying a fluorophore. Oligos are detected with an HCR amplifier labeled with Alexa 647. The U6 loading control is detected with a different HCR probe pair triggering an orthogonal HCR amplifier carrying Alexa 488. The fluorescent HCR signal scales linearly with the abundance of the target molecule, enabling relative quantitation between lanes for a given band.⁷⁸ The northern blot of FIG. 10A probing for RNA X_{Q} demonstrates that cells expressing RNA X_{Q} protected by a 5′ PEL have a significantly higher abundance of RNA X_{Q} than cells expressing RNA X_{Q} without a PEL. Likewise, the northern blot of FIG. 10B probing for RNA X_{T} demonstrates that cells expressing RNA X_{T} protected by a 5′ PEL have a significantly higher abundance of RNA X_{T} than cells expressing RNA X_{T} without a PEL. For these experiments, HEK 293T cells were transfected with plasmid encoding either RNA X_{Q} or RNA X_{T} with or without 5′ PEL and the cells were lysed and analyzed via northern blot 24 hours post-transfection. For the ctrl lysate lane, cells were transfected with a plasmid encoding neither RNA X_{Q} nor RNA X_{T}. FIG. 10C quantifies the bands for RNA X_{Q} with and without PEL in FIG. 10 A (the quantified band locations are marked by rectangles in FIG. 10A). FIG. 10E quantifies the fold-change increase in abundance for RNA X_{Q} and RNA X_{T} with PEL protection, demonstrating ≈15× protection for RNA X_{Q} and ≈5× protection of RNA X_{T}. FIG. 10F depicts the sequences of RNAs X_{Q} and X_{T} with or without a 5′ PEL. Nucleotides that are upper case italic are designed. The plain “C” nucleotide is a cloning artifact. Lower case plain nucleotides are constrained by the hU6 terminator sequence.⁷⁴ Bold nucleotides represent PEL sequence constrained by a portion of an xrRNA sequence derived from Dengue (Dengue 4, NC_002640.1).¹ The RNA X_{Q} protected from degradation by a PEL in this study is the same trigger X_{Q} that enhanced nucleic acid synthetic biology performance in FIGS. 7, 8, and 9. The RNA X_{T} protected from degradation by a PEL in this study is the same trigger RNA X_{T} that enhanced the performance of nucleic acid synthetic biology in FIG. 8.

### Example—Using PELs to Protect RNAs from Exoribonuclease Digestion

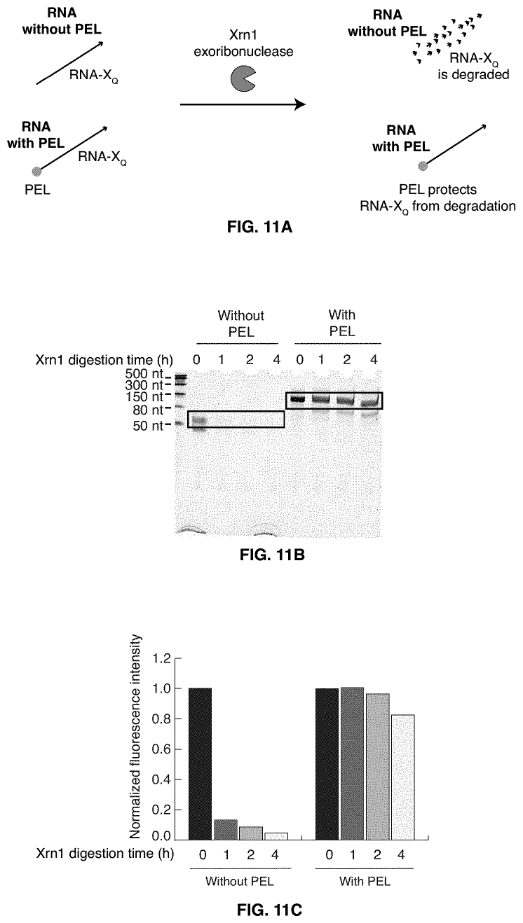

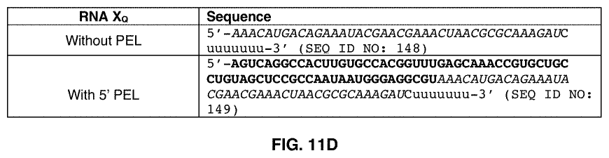

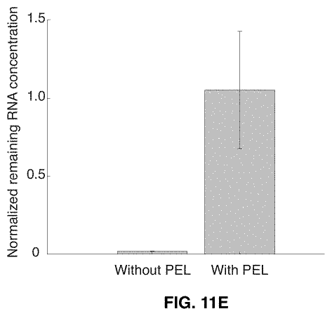

FIG. 11 demonstrates that a PEL protects RNA from digestion by 5′→3′ exoribonuclease Xrn1 which is an important enzyme in normal RNA decay pathways that degrade 5′ monophosphorylated RNAs⁷⁹. FIG. 11A depicts Xrn1 digestion of synthetic RNA X_{Q} synthesized with or without a 5′ PEL. FIG. 11B displays polyacrylamide gel electrophoresis showing that synthetic RNA X_{Q} (with or without PEL) incubated with Xrn1 and the activating enzyme RppH (digestion for a period of 0, 1, 2 or 4 hours) is quickly degraded without a 5′ PEL but is significantly protected by a 5′ PEL. FIG. 11C quantifies the RNA X_{Q} band in each lane (quantified region depicted in FIG. 11B), demonstrating that ˜80% of RNA X_{Q} remains after 4 hours with PEL protection, but less than 20% of RNA X_{Q} remains after 1 hour without PEL protection. FIG. 11E quantifies the remaining synthetic RNA X_{Q} (with or without PEL) after a 2 hour incubation with Xrn1 and the activating enzyme RppH as measured using quantitative reverse transcription PCR (RT-qPCR). Synthetic RNA X_{Q} is almost completely degraded without PEL protection but is significantly protected by a 5′ PEL. The bar graphs of FIG. 11E depict mean±estimated standard error of the mean (N=3 replicate experiments) for remaining RNA concentration normalized to undegraded RNA samples. FIG. 11D depicts the sequences of RNA X_{Q} with or without a 5′ PEL. Nucleotides that are upper case italic are designed. The plain “C” nucleotide is a cloning artifact. Lower case plain nucleotides are constrained by the hU6 terminator sequence.⁷⁴ Bold nucleotides are constrained by an PEL sequence from Dengue (Dengue 4, NC_002640.1).¹ The RNA X_{Q} protected from degradation by a PEL in this study is the same trigger X_{Q} that enhanced nucleic acid synthetic biology performance in FIGS. 7, 8, and 9.

### Example—Using a PEL to Block Exonuclease Digestion of the Portion of an RNA that is 3′ of the PEL

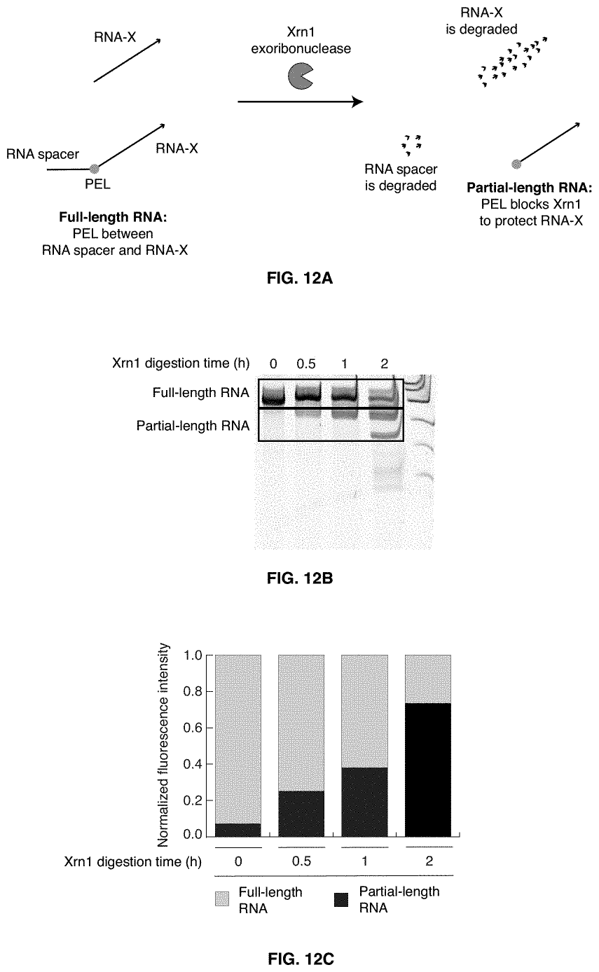

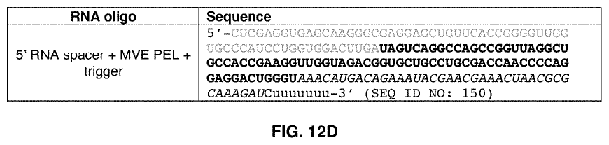

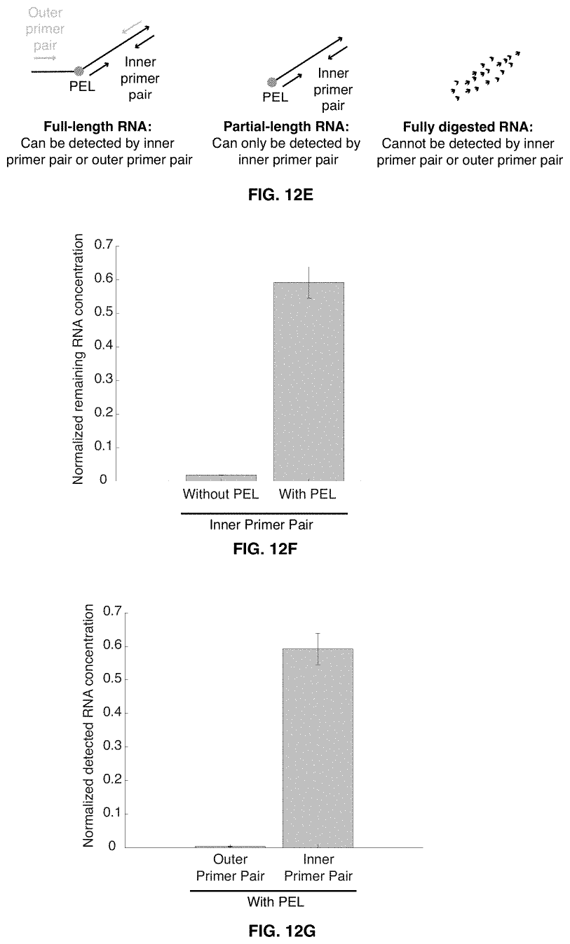

FIG. 12 demonstrates that a PEL forms a mechanical block to halt exoribonuclease Xrn1 from digesting RNA that is 3′ of the PEL. FIG. 12A depicts Xrn1 digestion of a synthetic RNA synthesized with 5′ RNA spacer+PEL+RNA X_{Q}. FIG. 12B displays polyacrylamide gel electrophoresis showing that over the course of 0, 0.5, 1, or 2 hours of Xrn1 digestion, the synthetic RNA shifts from predominantly a full-length RNA band to partial-length RNA bands, with the PEL blocking Xrn1 digestion of RNA X_{Q} which is 3′ of the PEL. FIG. 12C quantifies the full-length and partial-length RNA bands from the gel of FIG. 12B, confirming the shift from predominantly full-length to predominantly partial-length RNAs over the course of 2 hours for Xrn1 digestion. This demonstration illustrates that a PEL can be used to protect one portion of an RNA while leaving another portion of an RNA susceptible to degradation, enabling differential control over RNA durability in synthetic biology applications. FIG. 12E depicts the RT-qPCR primer pairs that can be used to distinguish between full-length RNAs, partial-length RNAs, and fully-digested RNAs: full-length RNAs can be detected with either an outer primer pair or an inner primer pair but partial-length RNAs (with the 5′ RNA degraded) can only be detected by the inner primer pair, and fully-digested RNAs cannot be detected by either primer pair. FIG. 12F uses the inner primer pair and RT-qPCR to quantify the amount of RNA X_{Q} that remains after a 2-hour incubation of synthetic RNA (with PEL: 5′ RNA spacer+PEL+RNA X_{Q}, or without PEL: 5′ RNA spacer+RNA X_{Q}) with Xrn1 and the activating enzyme RppH. Synthetic RNA RNA X_{Q} is predominantly degraded without PEL protection but is significantly protected by a 5′ PEL. FIG. 12G uses the synthetic RNA with PEL (5′ RNA spacer+PEL+RNA X_{Q}) and either the outer primer pair or inner primer pair with RT-qPCR to quantify the amount of full-length RNA remaining (using the outer primer pair) and the amount of partial length RNA remaining (using the inner primer pair) after a 2-hour incubation with Xrn1 and the activating enzyme RppH. The 5′ RNA spacer is almost completely degraded (measured using the outer primer pair) but the PEL substantially protects RNA X_{Q} (measured using the inner primer pair). The bar graphs of FIGS. 12F and 12G depict mean±estimated standard error of the mean (N=3 replicate experiments) for remaining RNA concentration normalized to undegraded RNA samples. FIG. 12D depicts the sequences of the synthetic RNA with 5′ spacer+PEL+RNA X_{Q}. Nucleotides that are gray represent the RNA spacer. Nucleotides that are upper case italic are designed. The plain “C” nucleotide is a cloning artifact. Lower case plain nucleotides are constrained by the hU6 terminator sequence.⁷⁴ Bold nucleotides represent a PEL sequence constrained by a portion of an xrRNA sequence derived from Dengue (Dengue 4, NC_002640.1).¹ The RNA X_{Q} protected from degradation by a PEL in this study is the same trigger X_{Q} that enhanced nucleic acid synthetic biology performance in FIGS. 7, 8, and 9.

### Example—Using Different PELs to Protect RNA from Exoribonuclease Digestion

FIGS. 13 and 29 demonstrate numerous PELs that protect RNA from digestion by exoribonuclease Xrn1. FIG. 13A depicts the experimental setup for incubation of Xrn1 with a synthetic RNA X_{Q} with or without protection by a 5′ PEL. FIG. 13B displays polyacrylamide gel electrophoresis showing that synthetic RNA X_{Q} (with or without PEL) incubated with Xrn1 and the activating enzyme RppH (digestion for a period of 0, 0.5, 1, or 2 hours) is rapidly degraded without a PEL but is significantly protected by any of a variety of 5′ PELs. FIG. 13C quantifies the RNA X_{Q} band in each lane (quantified region depicted in FIG. 13B), demonstrating that ˜90% of RNA X_{Q} remains after 2 hours with PEL protection, but less than 50% of RNA X_{Q} remains after 2 hours without PEL protection. FIG. 13D depicts the sequences of trigger X_{Q} with or without a 5′ PEL that were used for the experiments of FIGS. 13B-13C. PEL variants are derived from different viruses (MVE, Dengue 4, and Yellow fever virus). The RNA X_{Q} protected from degradation by a PEL in this study, and the PELs MVE-1, MVE-2, and Dengue are the same RNA components that enhanced nucleic acid synthetic biology performance in FIG. 9. For a number of different PELs, FIGS. 29A-29C quantify the remaining synthetic RNA X_{Q} (with or without PEL) after a 2 hour incubation with Xrn1 and the activating enzyme RppH as measured using quantitative reverse transcription PCR (RT-qPCR). Synthetic RNA X_{Q} is almost completely degraded without PEL protection but is significantly protected by any of a variety of different 5′ PELs: 12 different PEL motifs derived from 5 different viruses (FIG. 29A), 6 different PEL motifs derived from 6 different viruses (FIG. 29B), 12 different PEL motifs derived from 10 different viruses and 3 different PEL motifs that were designed computationally (FIG. 29C). The bar graphs of FIGS. 29A-29C depict mean±estimated standard error of the mean (N=3 replicate experiments) for remaining RNA concentration normalized to undegraded RNA samples. FIG. 29D depicts the sequences of trigger X_{Q} with or without a 5′ PEL that were used for the experiments of FIGS. 29A-29C. In FIGS. 13D and 29D, nucleotides that are upper case italic are rationally designed. The plain “C” nucleotide is a cloning artifact. Lower case plain nucleotides are constrained by the hU6 terminator sequence.⁷⁴ Bold nucleotides in FIG. 13D are PEL sequences constrained by a portion of an xrRNA sequence derived from: Murray Valley encephalitis (MVE, NC_000943.0,^{1,8} Dengue (Dengue 4, NC_002640.1),¹ or Yellow fever virus (YF, NC_002031.1).¹ Bold nucleotides in FIG. 29D represent either: Yellow fever virus (YF, NC_002031.1),¹ Dengue (Dengue 4, NC_002640.1),¹ Zika (NC_012532.1),¹ West Nile virus (WNV, NC_001563.2),¹ Murray Valley encephalitis (MVE, NC_000943.0,^{1,8} Opium poppy mosaic virus (OPMV),⁷ Potato leafroll virus (PLRV),⁷ Modoc virus (MODV),⁶ Tamana bat virus (TABV-1),⁴ Culex flavivirus (CXFV),⁴ Montana myotis leukoencephalitis virus (MMLV),⁶ Apoi virus (APOIV),⁶ Wesselbron,¹ Chaoyang,¹ Cell fusing agent virus (CFAV),⁶ Culex flavivirus (CXFV),⁴ Red clover necrotic mosaic virus (RCNMV),⁷¹ Sweet clover necrotic mosaic virus (SCNMV),⁷¹ Carnation ringspot virus (CRSV),⁷¹ or 2): a computationally designed riboswitch (Rbsw) sequence.⁷² Gray nucleotides in FIG. 29D represent a spacer sequence 5′ of the PEL. FIGS. 13E and 29E describe the pseudoknot and hairpin motifs used in each PEL motif. FIG. 13F and FIG. 29F depict the secondary structure of the pseudoknot and hairpin motifs used in each PEL motif.

### REFERENCES

(1) Kieft, J. S.; Rabe, J. L.; Chapman, E. G. New Hypotheses Derived from the Structure of a Flaviviral Xrn1-Resistant RNA: Conservation, Folding, and Host Adaptation. RNA Biol 2015, 12 (11), 1169-1177. https://doi.org/10.1080/15476286.2015.1094599.
(2) Pijlman, G. P.; Funk, A.; Kondratieva, N.; Leung, J.; Torres, S.; van der Aa, L.; Liu, W. J.; Palmenberg, A. C.; Shi, P.-Y.; Hall, R. A.; Khromykh, A. A. A Highly Structured, Nuclease-Resistant, Noncoding RNA Produced by Flaviviruses Is Required for Pathogenicity. Cell Host Microbe 2008, 4 (6), 579-591. https://doi.org/10.1016/j.chom.2008.10.007.
(3) Chapman, E. G.; Costantino, D. A.; Rabe, J. L.; Moon, S. L.; Wilusz, J.; Nix, J. C.; Kieft, J. S. The Structural Basis of Pathogenic Subgenomic Flavivirus RNA (SfRNA) Production. Science 2014, 344 (6181), 307-310. https://doi.org/10.1126/science.1250897.
(4) Szucs, M. J.; Nichols, P. J.; Jones, R. A.; Vicens, Q.; Kieft, J. S. A New Subclass of Exoribonuclease-Resistant RNA Found in Multiple Genera of Flaviviridae. 2020, 11 (5), 15.
(5) Jones, R. A.; Steckelberg, A.-L.; Vicens, Q.; Szucs, M. J.; Akiyama, B. M.; Kieft, J. S. Different Tertiary Interactions Create the Same Important 3D Features in a Distinct Flavivirus XrRNA. 13.
(6) MacFadden, A.; O'Donoghue, Z.; Silva, P. A. G. C.; Chapman, E. G.; Olsthoorn, R. C.; Sterken, M. G.; Pijlman, G. P.; Bredenbeek, P. J.; Kieft, J. S. Mechanism and Structural Diversity of Exoribonuclease-Resistant RNA Structures in Flaviviral RNAs. Nat. Commun. 2018, 9 (1), 119. https://doi.org/10.1038/s41467-017-02604-y.
(7) Steckelberg, A.-L.; Vicens, Q.; Kieft, J. S. Exoribonuclease-Resistant RNAs Exist within Both Coding and Noncoding Subgenomic RNAs. mBio 2018, 9 (6), e02461-18, /mbio/9/6/mBio.02461-18. atom. https://doi.org/10.1128/mBio.02461-18.
(8) Boehm, V.; Gerbracht, J. V.; Marx, M.-C.; Gehring, N. H. Interrogating the Degradation Pathways of Unstable MRNAs with XRN1-Resistant Sequences. Nat. Commun. 2016, 7 (1), 13691. https://doi.org/10.1038/ncomms13691.
(9) Voigt, F.; Gerbracht, J. V.; Boehm, V.; Horvathova, I.; Eglinger, J.; Chao, J. A.; Gehring, N. H. Detection and Quantification of RNA Decay Intermediates Using XRN1-Resistant Reporter Transcripts. Nat. Protoc. 2019, 14 (5), 1603-1633. https://doi.org/10.1038/s41596-019-0152-8.
(10) Bath, J.; Turberfield, A. J. DNA Nanomachines. Nat Nanotechnol 2007, 2, 275-284.
(11) Zhang, D. Y. Cooperative Hybridization of Oligonucleotides. J Am Chem Soc 2011, 133 (4), 1077-1086. https://doi.org/Doi 10.1021/Ja109089q.
(12) Yurke, B.; Turberfield, A. J.; Mills, Jr., A. P.; Simmel, F. C.; Neumann, J. L. A DNA-Fueled Molecular Machine Made of DNA. Nature 2000, 406 (6796), 605-608.
(13) Dirks, R. M.; Pierce, N. A. Triggered Amplification by Hybridization Chain Reaction. Proc. Natl. Acad. Sci. U.S.A 2004, 101 (43), 15275-15278.
(14) Seelig, G.; Soloveichik, D.; Zhang, D. Y.; Winfree, E. Enzyme-Free Nucleic Acid Logic Circuits. Science 2006, 314 (5805), 1585-1588.
(15) Venkataraman, S.; Dirks, R. M.; Rothemund, P. W. K.; Winfree, E.; Pierce, N. A. An Autonomous Polymerization Motor Powered by DNA Hybridization. Nat Nanotechnol 2007, 2, 490-494.
(16) Yin, P.; Choi, H. M. T.; Calvert, C. R.; Pierce, N. A. Programming Biomolecular Self-Assembly Pathways. Nature 2008, 451 (7176), 318-322. https://doi.org/10.1038/nature06451.
(17) Qian, L.; Winfree, E. Scaling Up Digital Circuit Computation with DNA Strand Displacement Cascades. Science 2011, 332 (6034), 1196-1201. https://doi.org/Doi 10.1126/Science.1200520.
(18) Turberfield, A. J.; Mitchell, J. C.; Yurke, B.; Mills, Jr., A. P.; Blakey, M. I.; Simmel, F. C. DNA Fuel for Free-Running Nanomachines. Phys. Rev. Lett. 2003, 90 (11), 118102.
(19) Hochrein, L. M.; Schwarzkopf, M.; Shahgholi, M.; Yin, P.; Pierce, N. A. Conditional Dicer Substrate Formation via Shape and Sequence Transduction with Small Conditional RNAs. J Am. Chem. Soc. 2013, 135 (46), 17322-17330.
(20) Bois, J. S.; Venkataraman, S.; Choi, H. M. T.; Spakowitz, A. J.; Wang, Z.-G.; Pierce, N. A. Topological Constraints in Nucleic Acid Hybridization Kinetics. Nucleic Acids Res. 2005, 33 (13), 4090-4095.
(21) Zhang, D. Y.; Turberfield, A. J.; Yurke, B.; Winfree, E. Engineering Entropy-Driven Reactions and Networks Catalyzed by DNA. Science 2007, 318 (5853), 1121-1125. https://doi.org/papers://865820FC-FC44-4379-A720-006E449021CA/Paper/p1718.
(22) Zhang, D. Y.; Winfree, E. Control of DNA Strand Displacement Kinetics Using Toehold Exchange. J. Am. Chem. Soc. 2009, 131, 17303-17314.
(23) Seelig, G.; Yurke, B.; Winfree, E. Catalyzed Relaxation of a Metastable DNA Fuel. J. Am. Chem. Soc. 2006, 128 (37), 12211-12220.
(24) Dabby, N. L. Synthetic Molecular Machines for Active Self-Assembly: Prototype Algorithms, Designs, and Experimental Study. thesis, 2013.
(25) Hanewich-Hollatz, M. H.; Chen, Z.; Hochrein, L. M.; Huang, J.; Pierce, N. A. Conditional Guide RNAs: Programmable Conditional Regulation of CRISPR/Cas Function in Bacterial and Mammalian Cells via Dynamic RNA Nanotechnology. ACS Cent. Sci. 2019, 5 (7), 1241-1249.
(26) Hochrein, L. M.; Ge, T. J.; Schwarzkopf, M.; Pierce, N. A. Signal Transduction in Human Cell Lysate via Dynamic RNA Nanotechnology. ACS Synth. Biol. 2018, 7 (12), 2796-2802.
(27) Siu, K.-H.; Chen, W. Riboregulated Toehold-Gated GRNA for Programmable CRISPR-Cas9 Function. Nat. Chem. Biol. 2019, 15 (3), 217-220. https://doi.org/10.1038/s41589-018-0186-1.
(28) Oesinghaus, L.; Simmel, F. C. Switching the Activity of Cas12a Using Guide RNA Strand Displacement Circuits. Nat. Commun. 2019, 10 (1), 1-11. https://doi.org/10.1038/s41467-019-09953-w.
(29) Ying, Z.-M.; Wang, F.; Chu, X.; Yu, R.-Q.; Jiang, J.-H. Activatable CRISPR Transcriptional Circuits Generate Functional RNA for MRNA Sensing and Silencing. Angew. Chem. Int. Ed. 2020, 59, 18599-18604.
(30) Pinheiro, A. V.; Han, D. R.; Shih, W. M.; Yan, H. Challenges and Opportunities for Structural DNA Nanotechnology. Nat. Nanotechnol. 2011, 6 (12), 763-772. https://doi.org/Doi 10.1038/Nnano.2011.187.
(31) Zhang, D. Y.; Seelig, G. Dynamic DNA Nanotechnology Using Strand-Displacement Reactions. Nat Chem 2011, 3 (2), 103-113. https://doi.org/Doi 10.1038Nchem.957.
(32) Isaacs, F. J.; Dwyer, D. J.; Collins, J. J. RNA Synthetic Biology. Nat. Biotechnol. 2006, 24 (5), 545-554. https://doi.org/10.1038/nbt1208.
(33) Chappell, J.; Watters, K. E.; Takahashi, M. K.; Lucks, J. B. A Renaissance in RNA Synthetic Biology: New Mechanisms, Applications and Tools for the Future. Curr Opin Chem Biol 2015, 28, 47-56.
(34) Chappell, J.; Takahashi, M. K.; Meyer, S.; Loughrey, D.; Watters, K. E.; Lucks, J. The Centrality of RNA for Engineering Gene Expression. Biotechnol. J. 2013, 8 (12), 1379-1395. https://doi.org/Doi 10.1002/Biot.201300018.
(35) Chappell, J.; Takahashi, M. K.; Lucks, J. B. Creating Small Transcription Activating RNAs. Nat. Chem. Biol. 2015, 11 (3), 214-220. https://doi.org/10.1038/nchembio.1737.
(36) Meyer, S.; Chappell, J.; Sankar, S.; Chew, R.; Lucks, J. B. Improving Fold Activation of Small Transcription Activating RNAs (STARs) with Rational RNA Engineering Strategies: Improving Small Transcription Activating RNAs. Biotechnol. Bioeng. 2016, 113 (1), 216-225. https://doi.org/10.1002/bit.25693.
(37) Green, A. A.; Silver, P. A.; Collins, J. J.; Yin, P. Toehold Switches: De-Novo-Designed Regulators of Gene Expression. Cell 2014, 159 (4), 925-939.
(38) Qi, L. S.; Larson, M. H.; Gilbert, L. A.; Doudna, J. A.; Weissman, J. S.; Arkin, A. P.;
Lim, W. A. Repurposing CRISPR as an RNA-Guided Platform for Sequence-Specific Control of Gene Expression. Cell 2013, 152 (5), 1173-1183. https://doi.org/10.1016/j.cell.2013.02.022.
(39) Gilbert, L. A.; Larson, M. H.; Morsut, L.; Liu, Z.; Brar, G. A.; Torres, S. E.; Stern-Ginossar, N.; Brandman, O.; Whitehead, E. H.; Doudna, J. A.; Lim, W. A.; Weissman, J. S.; Qi, L. S. CRISPR-Mediated Modular RNA-Guided Regulation of Transcription in Eukaryotes. Cell 2013, 154 (2), 442-451.
(40) Zalatan, J. G.; Lee, M. E.; Almeida, R.; Gilbert, L. A.; Whitehead, E. H.; La Russa, M.; Tsai, J. C.; Weissman, J. S.; Dueber, J. E.; Qi, L. S.; Lim, W. A. Engineering Complex Synthetic Transcriptional Programs with CRISPR RNA Scaffolds. Cell 2015, 160 (1-2), 339-350. https://doi.org/10.1016/j.cell.2014.11.052.
(41) Ma, H.; Tu, L.-C.; Naseri, A.; Huisman, M.; Zhang, S.; Grunwald, D.; Pederson, T. Multiplexed Labeling of Genomic Loci with DCas9 and Engineered SgRNAs Using CRISPRainbow. Nat. Biotechnol. 2016, 34 (5), 528-530. https://doi.org/10.1038/nbt.3526.
(42) Liu, X. S.; Wu, H.; Krzisch, M.; Wu, X.; Graef, J.; Muffat, J.; Hnisz, D.; Li, C. H.; Yuan, B.; Xu, C.; Li, Y.; Vershkov, D.; Cacace, A.; Young, R. A.; Jaenisch, R. Rescue of Fragile X Syndrome Neurons by DNA Methylation Editing of the FMR1 Gene. Cell 2018, 172 (5), 979-992.e6. https://doi.org/10.1016/j.cell.2018.01.012.
(43) Myers, S. A.; Wright, J.; Peckner, R.; Kalish, B. T.; Zhang, F.; Carr, S. A. Discovery of Proteins Associated with a Predefined Genomic Locus via DCas9-APEX-Mediated Proximity Labeling. Nat. Methods 2018, 15 (6), 437-439. https://doi.org/10.1038/s41592-018-0007-1.
(44) Morgan, S. L.; Mariano, N. C.; Bermudez, A.; Arruda, N. L.; Wu, F.; Luo, Y.; Shankar, G.; Jia, L.; Chen, H.; Hu, J.-F.; Hoffman, A. R.; Huang, C.-C.; Pitteri, S. J.; Wang, K. C. Manipulation of Nuclear Architecture through CRISPR-Mediated Chromosomal Looping. Nat. Commun. 2017, 8 (1), 15993. https://doi.org/10.1038/ncomms15993.
(45) Rothemund, P. W. K. Folding DNA to Create Nanoscale Shapes and Patterns. Nature 2006, 440 (7082), 297-302.
(46) Geary, C.; Rothemund, P. W. K.; Andersen, E. S. A Single-Stranded Architecture for Cotranscriptional Folding of RNA Nanostructures. Science 2014, 345 (6198), 799-804.
(47) Delebecque, C. J.; Lindner, A. B.; Silver, P. A.; Aldaye, F. A. Organization of Intracellular Reactions with Rationally Designed RNA Assemblies. Science 2011, 333 (6041), 470-474. https://doi.org/10.1126/science.1206938.
(48) Delebecque, C. J.; Silver, P. A.; Lindner, A. B. Designing and Using RNA Scaffolds to Assemble Proteins in Vivo. Nat. Protoc. 2012, 7 (10), 1797-1807. https://doi.org/Doi 10.1038Nprot.2012.102.
(49) Pardee, K.; Green, A. A.; Ferrante, T.; Cameron, D. E.; DaleyKeyser, A.; Yin, P.; Collins, J. J. Paper-Based Synthetic Gene Networks. Cell 2014, 159 (4), 940-954. https://doi.org/10.1016/j.cell.2014.10.004.
(50) Pardee, K.; Green, A. A.; Takahashi, M. K.; Braff, D.; Lambert, G.; Lee, J. W.; Ferrante, T.; Ma, D.; Donghia, N.; Fan, M.; Daringer, N. M.; Bosch, I.; Dudley, D. M.; O'Connor, D. H.; Gehrke, L.; Collins, J. J. Rapid, Low-Cost Detection of Zika Virus Using Programmable Biomolecular Components. Cell 2016, 165 (5), 1255-1266. https://doi.org/10.1016/j.cell.2016.04.059.
(51) Pardi, N.; Hogan, M. J.; Porter, F. W.; Weissman, D. MRNA Vaccines—a New Era in Vaccinology. Nat. Rev. Drug Discov. 2018, 17 (4), 261-279. https://doi.org/10.1038/nrd.2017.243.
(52) Sahin, U.; Karikó, K.; Türeci, Ö. MRNA-Based Therapeutics—Developing a New Class of Drugs. Nat. Rev. Drug Discov. 2014, 13 (10), 759-780. https://doi.org/10.1038/nrd4278.
(53) Kim, T.; Lu, T. K. CRISPR/Cas-Based Devices for Mammalian Synthetic Biology. Curr. Opin. Chem. Biol. 2019, 52, 23-30. https://doi. org/10.1016/j.cbpa.2019.04.015.
(54) Moon, S. B.; Kim, D. Y.; Ko, J.-H.; Kim, J.-S.; Kim, Y.-S. Improving CRISPR Genome Editing by Engineering Guide RNAs. Trends Biotechnol. 2019, 37 (8), 870-881. https://doi. org/10.1016/j.tibtech.2019.01.009.
(55) Kim, Y.-K. RNA Therapy: Current Status and Future Potential. Chonnam Med. J. 2020, 56 (2), 87. https://doi.org/10.4068/cmj.2020.56.2.87.
(56) Yu, A.-M.; Choi, Y. H.; Tu, M.-J. RNA Drugs and RNA Targets for Small Molecules: Principles, Progress, and Challenges. Pharmacol. Rev. 2020, 72 (4), 862-898. https://doi.org/10.1124/pr.120.019554.
(57) Bumcrot, D.; Manoharan, M.; Koteliansky, V.; Sah, D. W. Y. RNAi Therapeutics: A Potential New Class of Pharmaceutical Drugs. Nat. Chem. Biol. 2006, 2 (12), 711-719.
(58) Elmen, J.; Thonberg, H.; Ljungberg, K.; Frieden, M.; Westergaard, M.; Xu, Y.; Wahren, B.; Liang, Z.; Orum, H.; Koch, T.; Wahlestedt, C. Locked Nucleic Acid (LNA) Mediated Improvements in SiRNA Stability and Functionality. Nucleic Acids Res. 2005, 33 (1), 439-447.
(59) Judge, A. D.; Bola, G.; Lee, A. C.; MacLachlan, I. Design of Noninflammatory Synthetic SiRNA Mediating Potent Gene Silencing in Vivo. Mol. Ther. 2006, 13 (3), 494-505.
(60) Manoharan, M. RNA Interference and Chemically Modified Small Interfering RNAs. Curr Opin Chem Biol 2004, 8 (6), 570-579. https://doi.org/S1367-5931(04)00138-3 [pii] 10.1016/j.cbpa.2004.10.007.
(61) Czauderna, F.; Fechtner, M.; Dames, S.; Aygun, H.; Klippel, A.; Pronk, G. J.; Giese, K.; Kaufmann, J. Structural Variations and Stabilising Modifications of Synthetic SiRNAs in Mammalian Cells. Nucleic Acids Res 2003, 31 (11), 2705-2716.
(62) Layzer, J. M.; McCaffrey, A. P.; Tanner, A. K.; Huang, Z.; Kay, M. A.; Sullenger, B. A. In Vivo Activity of Nuclease-Resistant SiRNAs. Rna 2004, 10 (5), 766-771.
(63) Collingwood, M. A.; Rose, S. D.; Huang, L. Y.; Hillier, C.; Amarzguioui, M.; Wiiger, M. T.; Soifer, H. S.; Rossi, J. J.; Behlke, M. A. Chemical Modification Patterns Compatible with High Potency Dicer-Substrate Small Interfering RNAs. Oligonucleotides 2008, 18 (2), 187-199.
(64) Glick, B. R. Metabolic Load and Heterologous Gene Expression. Biotechnol. Adv. 1995, 13 (2), 247-261. https://doi.org/10.1016/0734-9750(95)00004-A.
(65) Carbonell-Ballestero, M.; Garcia-Ramallo, E.; Montañez, R.; Rodriguez-Caso, C.; Macia, J. Dealing with the Genetic Load in Bacterial Synthetic Biology Circuits: Convergences with the Ohm's Law. Nucleic Acids Res. 2016, 44 (1), 496-507. https://doi.org/10.1093/nar/gkv1280.
(66) Wu, G.; Yan, Q.; Jones, J. A.; Tang, Y. J.; Fong, S. S.; Koffas, M. A. G. Metabolic Burden: Cornerstones in Synthetic Biology and Metabolic Engineering Applications. Trends Biotechnol. 2016, 34 (8), 652-664. https://doi.org/10.1016/j.tibtech.2016.02.010.
(67) Decroly, E.; Ferron, F.; Lescar, J.; Canard, B. Conventional and Unconventional Mechanisms for Capping Viral MRNA. Nat. Rev. Microbiol. 2012, 10 (1), 51-65. https://doi.org/10.1038/nrmicro2675.
(68) Paterson, B. M.; Rosenberg, M. Efficient Translation of Prokaryotic MRNAs in a Eukaryotic Cell-Free System Requires Addition of a Cap Structure. Nature 1979, 279 (5715), 692-696. https://doi.org/10.1038/279692a0.
(69) Zadeh, J. N.; Steenberg, C. D.; Bois, J. S.; Wolfe, B. R.; Pierce, M. B.; Khan, A. R.; Dirks, R. M.; Pierce, N. A. NUPACK: Analysis and Design of Nucleic Acid Systems. J. Comput. Chem. 2011, 32 (1), 170-173. https://doi.org/10.1002/jcc.21596.
(70) Wolfe, B. R.; Porubsky, N. J.; Zadeh, J. N.; Dirks, R. M.; Pierce, N. A. Constrained Multistate Sequence Design for Nucleic Acid Reaction Pathway Engineering. J. Am. Chem. Soc. 2017, 139 (8), 3134-3144.
(71) Steckelberg, A.-L.; Akiyama, B. M.; Costantino, D. A.; Sit, T. L.; Nix, J. C.; Kieft, J. S. A Folded Viral Noncoding RNA Blocks Host Cell Exoribonucleases through a Conformationally Dynamic RNA Structure. Proc. Natl. Acad. Sci. 2018, 115 (25), 6404-6409. https://doi.org/10.1073/pnas.1802429115.
(72) Roth, A.; Winkler, W. C.; Regulski, E. E.; Lee, B. W. K.; Lim, J.; Jona, I.; Barrick, J. E.; Ritwik, A.; Kim, J. N.; Welz, R.; Iwata-Reuyl, D.; Breaker, R. R. A Riboswitch Selective for the Queuosine Precursor PreQ1 Contains an Unusually Small Aptamer Domain. Nat. Struct. Mol. Biol. 2007, 14 (4), 308-317. https://doi.org/10.1038/nsmb1224.
(73) Kieft, J. S.; Rabe, J. L.; Chapman, E. G. New Hypotheses Derived from the Structure of a Flaviviral Xrn1-Resistant RNA: Conservation, Folding, and Host Adaptation. RNA Biol. 2015, 12 (11), 1169-1177. https://doi.org/10.1080/15476286.2015.1094599.
(74) Gao, Z.; Herrera-Carrillo, E.; Berkhout, B. Delineation of the Exact Transcription Termination Signal for Type 3 Polymerase III. Mol. Ther. Nucleic Acids 2018, 10, 36-44.
(75) Chavez, A.; Scheiman, J.; Vora, S.; Pruitt, B. W.; Tuttle, M.; Iyer, E. P. R.; Lin, S.; Kiani, S.; Guzman, C. D.; Wiegand, D. J.; Ter-Ovanesyan, D.; Braff, J. L.; Davidsohn, N.; Housden, B. E.; Perrimon, N.; Weiss, R.; Aach, J.; Collins, J. J.; Church, G. M. Highly Efficient Cas9-Mediated Transcriptional Programming. Nat. Methods 2015, 12 (4), 326-328. https://doi.org/10.1038/nmeth.3312.
(76) Shagin, D. A.; Barsova, E. V.; Yanushevich, Y. G.; Fradkov, A. F.; Lukyanov, K. A.; Labas, Y. A.; Semenova, T. N.; Ugalde, J. A.; Meyers, A.; Nunez, J. M.; Widder, E. A.; Lukyanov, S. A.; Matz, M. V. GFP-like Proteins as Ubiquitous Metazoan Superfamily: Evolution of Functional Features and Structural Complexity. Mol. Biol. Evol. 2004, 21 (5), 841-850. https://doi.org/10.1093/molbev/msh079.0
(77) Nissim, L.; Perli, S. D.; Fridkin, A.; Perez-Pinera, P.; Lu, T. K. Multiplexed and Programmable Regulation of Gene Networks with an Integrated RNA and CRISPR/Cas Toolkit in Human Cells. Mol Cell 2019, 54 (4), 698-710.
(78) Schwarzkopf, M.; Pierce, N. A. Multiplexed MiRNA Northern Blots via Hybridization Chain Reaction. Nucleic Acids Res. 2016, 44 (15), e129. https://doi.org/10.1093/nar/gkw503.
(79) Jones, C. I.; Zabolotskaya, M. V.; Newbury, S. F. The 5′→3′ Exoribonuclease XRN1/Pacman and Its Functions in Cellular Processes and Development: The 5′→3′ Exoribonuclease XRN1/Pacman and Its Functions. Wiley Interdiscip. Rev. RNA 2012, 3 (4), 455-468. https://doi.org/10.1002/wrna.1109.

1. A protective element (PEL) within a synthesized or expressed RNA molecule that reduces degradation of a sequence element 5′ and/or 3′ of the PEL, wherein the sequence element that experiences reduced degradation is known as a protected sequence.

2. A protective element (PEL) within a nucleic acid, wherein the PEL comprises a structured region comprising one or more duplexes, and wherein the structured region reduces degradation of a protected sequence 5′ and/or 3′ of the PEL.

3. The PEL of claim 2, wherein the PEL comprises a PEL motif comprising a pseudoknot motif: the pseudoknot motif comprising (from 5′ to 3′) a 1^{st} segment, a 2^{nd} segment, a 3^{rd} segment, a 4^{th} segment, a 5^{th} segment, a 6^{th} segment, a 7^{th} segment, and an 8^{th} segment, wherein the 1^{st} segment hybridizes to the 7^{th} segment to form a 1^{st} duplex, the 2^{nd} segment hybridizes to the 3^{rd} segment to form a 2^{nd} duplex, the 4^{th} segment hybridizes to the 6^{th} segment to form a 3^{rd} duplex, and the 5^{th} segment hybridizes to the 8^{th} segment to form a 4^{th} duplex.

4. (canceled)

5. The PEL of claim 2, wherein the PEL comprises a PEL motif comprising (from 5′ to 3′) a pseudoknot motif and a hairpin motif:
a. the pseudoknot motif comprising (from 5′ to 3′) a 1^{st} segment, a 2^{nd} segment, a 3^{rd} segment, a 4^{th} segment, a 5^{th} segment, a 6^{th} segment, a 7^{th} segment, and an 8^{th} segment, wherein the 1^{st} segment hybridizes to the 7^{th} segment to form a 1^{st} duplex, the 2^{nd} segment hybridizes to the 3^{rd} segment to form a 2^{nd} duplex, the 4^{th} segment hybridizes to the 6^{th} segment to form a 3^{rd} duplex, the 5^{th} segment hybridizes to the 8^{th} segment to form a 4^{th} duplex; and
b. the hairpin motif comprising (from 5′ to 3′) a 9^{th} segment and a 10^{th} segment, wherein the 9^{th} segment hybridizes to the 10^{th} segment to form a 5^{th} duplex.

6. (canceled)

7. The PEL of claim 2, wherein the PEL comprises a PEL motif comprising (from 5′ to 3′) a first pseudoknot motif and a second pseudoknot motif:
a. the first pseudoknot motif comprising (from 5′ to 3′) a 1^{st} segment, a 2^{nd} segment, a 3^{rd} segment, a 4^{th} segment, a 5^{th} segment, a 6^{th} segment, a 7^{th} segment, and an 8^{th} segment, wherein the 1^{st} segment hybridizes to the 7^{th} segment to form a 1^{st} duplex, the 2^{nd} segment hybridizes to the 3^{rd} segment to form a 2^{nd} duplex, the 4^{th} segment hybridizes to the 6^{th} segment to form a 3^{rd} duplex, and the 5^{th} segment hybridizes to the 8^{th} segment to form a 4^{th} duplex; and
b. the second pseudoknot motif comprising (from 5′ to 3′) a 9^{th} segment, a 10^{th} segment, an 11^{th} segment, a 12^{th} segment, a 13^{th} segment, a 14^{th} segment, a 15^{th} segment, and a 16^{th} segment, wherein the 9^{th} segment hybridizes to the 15^{th} segment to form a 5^{th} duplex, the 10^{th} segment hybridizes to the 11^{th} segment to form a 6^{th} duplex, the 12^{th} segment hybridizes to the 14^{th} segment to form a 7^{th} duplex, and the 13^{th} segment hybridizes to the 16^{th} segment to form an 8^{th} duplex.

8. (canceled)

9. (canceled)

10. The PEL of claim 2, wherein the PEL comprises a PEL motif comprising (from 5′ to 3′) a first pseudoknot motif, a first hairpin motif, a second pseudoknot motif, and a second hairpin motif:
a. the first pseudoknot motif comprising (from 5′ to 3′) a Pt segment, a 2^{nd} segment, a 3^{rd} segment, a 4^{th} segment, a 5^{th} segment, a 6^{th} segment, a 7^{th} segment, and an 8^{th} segment, wherein the 1^{st} segment hybridizes to the 7^{th} segment to form a 1^{st} duplex, the 2^{nd} segment hybridizes to the 3^{rd} segment to form a 2^{nd} duplex, the 4^{th} segment hybridizes to the 6^{th} segment to form a 3^{rd} duplex, and the 5^{th} segment hybridizes to the 8^{th} segment to form a 4^{th} duplex;
b. the first hairpin motif comprising (from 5′ to 3′) a 9^{th} segment and a 10^{th} segment, wherein the 9^{th} segment hybridizes to the 10^{th} segment to form a 5^{th} duplex;
c. the second pseudoknot motif comprising (from 5′ to 3′) an 11^{th} segment, a 12^{th} segment, a 13^{th} segment, a 14^{th} segment, a 15^{th} segment, a 16^{th} segment, a 17^{th} segment, and an 18^{th} segment, wherein the 11^{th} segment hybridizes to the 17^{th} segment to form a 6^{th} duplex, the 12^{th} segment hybridizes to the 13^{th} segment to form a 7^{th} duplex, the 14^{th} segment hybridizes to the 16^{th} segment to form an 8^{th} duplex, and the 15^{th} segment hybridizes to the 18^{th} segment to form a 9^{th} duplex; and
d. the second hairpin motif comprising (from 5′ to 3′) a 19^{th} segment and a 20^{th} segment, wherein the 19^{th} segment hybridizes to the 20^{th} segment to form a 10^{th} duplex.

11. (canceled)

12. (canceled)

13. The PEL of claim 2, wherein the PEL comprises a PEL motif comprising a pseudoknot motif: the pseudoknot motif comprising (from 5′ to 3′) a 1st segment, a 2^{nd} segment, a 3^{rd} segment, a 4^{th} segment, a 5^{th} segment, a 6^{th} segment, a 7^{th} segment, an 8^{th} segment, a 9^{th} segment, and a 10^{th} segment, wherein the 1^{st} segment hybridizes to the 9^{th} segment to form a 1^{st} duplex, the 2^{nd} segment hybridizes to the 8^{th} segment to form a 2^{nd} duplex, the 3^{rd} segment hybridizes to the 4^{th} segment to form a 3^{rd} duplex, the 5^{th} segment hybridizes to the 7^{th} segment to form a 4^{th} duplex, and the 6^{th} segment hybridizes to the 10^{th} segment to form a 5^{th} duplex.

14. The PEL of claim 2, wherein the PEL comprises a PEL motif comprising a pseudoknot motif: the pseudoknot motif comprising (from 5′ to 3′) a 1^{st} segment, a 2^{nd} segment, a 3^{rd} segment, a 4^{th} segment, a 5^{th} segment, and a 6^{th} segment, wherein the 1^{st} segment hybridizes to the 5^{th} segment to form a 1^{st} duplex, the 2^{nd} segment hybridizes to the 4^{th} segment to form a 2^{nd} duplex, and the 3^{rd} segment hybridizes to the 6^{th} segment to form a 3rd duplex.

15. The PEL of claim 2, wherein the PEL comprises a PEL motif comprising a pseudoknot motif: the pseudoknot motif comprising (from 5′ to 3′) a 1^{st} segment, a 2^{nd} segment, a 3^{rd} segment, and a 4^{th} segment, wherein the 1^{st} segment hybridizes to the 3^{rd} segment to form a 1^{st} duplex and the 2^{nd} segment hybridizes to the 4^{th} segment to form a 2^{nd} duplex.

16. The PEL of claim 2, wherein the PEL comprises a PEL motif comprising a pseudoknot motif: the pseudoknot motif comprising (from 5′ to 3′) a 1^{st} segment, a 2^{nd} segment, a 3^{rd} segment, and a 4^{th} segment, wherein the 1^{st} segment hybridizes to the 3^{rd} segment to form a structured region comprising a 1^{st} duplex and the 2^{nd} segment hybridizes to the 4^{th} segment to form a 2^{nd} duplex.

17. (canceled)

18. The PEL of claim 2, wherein the PEL comprises a PEL motif comprising a pseudoknot motif: the pseudoknot motif comprising (from 5′ to 3′) a 1^{st} segment, a 2^{nd} segment, a 3^{rd} segment, a 4^{th} segment, a 5^{th} segment, and a 6^{th} segment, wherein the 1^{st} segment hybridizes to the 3^{rd} segment to form a 1^{st} structured region comprising a 1^{st} duplex, the 2^{nd} segment hybridizes to the 5^{th} segment to form a 2^{nd} duplex, and the 4^{th} segment hybridizes to the 6^{th} segment to form a 2^{nd} structured region comprising a 3^{rd} duplex.

19. The PEL of claim 2, wherein the PEL comprises a PEL motif comprising a pseudoknot motif: the pseudoknot motif comprising (from 5′ to 3′) a 1st segment, a 2^{nd} segment, a 3^{rd} segment, a 4^{th} segment, a 5^{th} segment, and a 6^{th} segment, wherein the 1^{st} segment hybridizes to the 3^{rd} segment to form a 1^{st} structured region comprising a 1^{st} duplex, the 2^{nd} segment hybridizes to the 5^{th} segment to form a 2^{nd} duplex, and a 3^{rd} duplex is formed within a 2^{nd} structured region by hybridization between two sub-segments of the 4^{th} segment or between two sub-segments of the 6^{th} segment.

20. (canceled)

21. The PEL of claim 2, wherein the PEL comprises a PEL motif comprising a pseudoknot motif: the pseudoknot motif comprising (from 5′ to 3′) a 1^{st} segment, a 2^{nd} segment, a 3^{rd} segment, and a 4^{th} segment, wherein the 1^{st} segment hybridizes to the 3^{rd} segment to form a 1^{st} duplex and the 2^{nd} segment hybridizes to the 4^{th} segment to form a structured region comprising a 2^{nd} duplex.

22. (canceled)

23. The PEL of claim 2, wherein the PEL comprises a PEL motif comprising a structured region, the structured region comprising a first duplex, wherein the structured region serves as a mechanical block to inhibit nuclease degradation of the protected sequence.

24.-48. (canceled)


SEQUENCE LISTING (221 sequences)

SEQ ID NO: 1 - RNA, 52 nt, Artificial Sequence
  Dengue1-1
agucaggccaggcaaugccugccaccggaaguuggaugacggccugugagcc

SEQ ID NO: 2 - RNA, 52 nt, Artificial Sequence
  Dengue2-1
agucaggucggauuaagccauaguacggaaaaaacuaugcuaccugugagcc

SEQ ID NO: 3 - RNA, 52 nt, Artificial Sequence
  Dengue3-1
ugucaggccaccuuaagccacaguacggaagaagcugugcugccugugagcc

SEQ ID NO: 4 - RNA, 51 nt, Artificial Sequence
  Kokobera-1
agucaggccugaaaagccaccugauccggugaaggugcugccugcauccgg

SEQ ID NO: 5 - RNA, 55 nt, Artificial Sequence
  Zika-1.2
ugucaggccugcuagucagccacaguuuggggaaagcugugcagccuguaacccc

SEQ ID NO: 6 - RNA, 60 nt, Artificial Sequence
  St. Louis encephalitis-1
agucaggccaaucaguuuugccaccggaugucagguaaacggugcugucuguaaccuggc

SEQ ID NO: 7 - RNA, 60 nt, Artificial Sequence
  Japanese encephalitis-1
agucaggccagcaaaagcugccaccggauacuggguagacggugcugucugcgucucagu

SEQ ID NO: 8 - RNA, 60 nt, Artificial Sequence
  Ntaya-1
agucaggccaggcaaugccugccaccggaaguuggaugacggugcugucugcgcuccaac

SEQ ID NO: 9 - RNA, 60 nt, Artificial Sequence
  Usutu-1
agucaggccagggcaaccugccaccggaaguugaguagacggugcugccugcgacucaac

SEQ ID NO: 10 - RNA, 61 nt, Artificial Sequence
  Murray Valley encephalitis-1.1
agucaggccagccgguuaggcugccaccgaagguugguagacggugcugccugcgaccaac

SEQ ID NO: 11 - RNA, 61 nt, Artificial Sequence
  West Nile-1
agucaggccagauuaaugcugccaccggaaguugaguagacggugcugccugcggcucaac

SEQ ID NO: 12 - RNA, 48 nt, Artificial Sequence
  Dengue1-2
agucaggccgaaagccacgguucgagcaagccgugcugccuguagcuc

SEQ ID NO: 13 - RNA, 53 nt, Artificial Sequence
  Dengue2-2
agucaggccaucauaaaugccauagcuugaguaaacuaugcagccuguagcuc

SEQ ID NO: 14 - RNA, 49 nt, Artificial Sequence
  Dengue3-2
agucaggccccaaagccacgguuugagcaaaccgugcugccuguagcuc

SEQ ID NO: 15 - RNA, 43 nt, Artificial Sequence
  Kokobera-2
agucagauccgaaaggccaccaguuuggugcagaacuggugcu

SEQ ID NO: 16 - RNA, 51 nt, Artificial Sequence
  Zika-2
agucaggccgagaacgccauggcacggaagaagccaugcugccugugagcc

SEQ ID NO: 17 - RNA, 54 nt, Artificial Sequence
  St. Louis encephalitis -2
agucagaccagaaaugccaccugaaagcaugcuaaaggugcugucuguacaugc

SEQ ID NO: 18 - RNA, 51 nt, Artificial Sequence
  Japanese encephalitis-2
cgucaggccacaaauuugugccaccccgcuagggggugcggccugcgcagc

SEQ ID NO: 19 - RNA, 57 nt, Artificial Sequence
  Ntaya-2
agucaggccguagguuuuacgccacuagcaugcagugcugccugagacaagacaugc

SEQ ID NO: 20 - RNA, 48 nt, Artificial Sequence
  Usutu-2
ugucaggccgcaaagcgccacuucgccaaggagugcagccuguacggc

SEQ ID NO: 21 - RNA, 49 nt, Artificial Sequence
  Murray Valley encephalitis-2
ugucagaucgcgaaagcgccacuucgccgaggagugcaaucugugaggc

SEQ ID NO: 22 - RNA, 55 nt, Artificial Sequence
  West Nile-2
ugucagaccacacuuuaaugugccacucugcggagagugcagucugcgauagugc

SEQ ID NO: 23 - RNA, 50 nt, Artificial Sequence
  Dengue4-1
agucaggccacuugugccacgguuugagcaaaccgugcugccuguagcuc

SEQ ID NO: 24 - RNA, 59 nt, Artificial Sequence
  Wesselbron-1
agucagcccaucauuugaugccauggcuaagcugugaggcccaugcuggcugggacagc

SEQ ID NO: 25 - RNA, 58 nt, Artificial Sequence
  Ilheus-1
ugucaggccauggaaacaugccacccaaagcuuguagagggugcagccugcgccaagc

SEQ ID NO: 26 - RNA, 59 nt, Artificial Sequence
  Sepik-1
ugucagcccgucauaaugacgccauggcuaagcugugaggccaugcuggcugggauagc

SEQ ID NO: 27 - RNA, 54 nt, Artificial Sequence
  Bussuquara-1
agucaggccagaaaugccaccggauaaagguagacggugcugccugcaaccuuu

SEQ ID NO: 28 - RNA, 60 nt, Artificial Sequence
  Tembusu-1
agucaggccagggaaucccugccaccggauguuggaugacggugcugucugcguuccaac

SEQ ID NO: 29 - RNA, 53 nt, Artificial Sequence
  Chaoyang-1
agucaggccuaaaugccaccggaugauaguagacggugcugccugcagcuauc

SEQ ID NO: 30 - RNA, 57 nt, Artificial Sequence
  Kedougou-1
cgucaggccacucgugagagugccacaguacgguaaagacugugcggccugcgagcc

SEQ ID NO: 31 - RNA, 59 nt, Artificial Sequence
  Yokose-1
ugucaggccaagauugagaaaaucuugccacagcuuggcagacugugcagccugcagcc

SEQ ID NO: 32 - RNA, 60 nt, Artificial Sequence
  Dongang-1
agucaggccucacgaaugugagccaccggaugggacuagacggugcugccugcgcguccc

SEQ ID NO: 33 - RNA, 67 nt, Artificial Sequence
  Yellow Fever-1
ugucagcccagaaccccacacgaguuuugccacugcuaagcugugaggcagugcaggcugggacagc

SEQ ID NO: 34 - RNA, 62 nt, Artificial Sequence
  Murray Valley encephalitis-1.2
uagucaggccagccgguuaggcugccaccgaagguugguagacggugcugccugcgaccaac

SEQ ID NO: 35 - RNA, 64 nt, Artificial Sequence
  Zika-1.2
ugucaggccugcuagucagccacaguuuggggaaagcugugcagccuguaacccccccaggaga

SEQ ID NO: 36 - RNA, 75 nt, Artificial Sequence
  Dengue1-3
agucaggccaggcaaugccugccaccggaaguuggaugacggugcugucugcgcuccaaccccaggcggacuggg

SEQ ID NO: 37 - RNA, 65 nt, Artificial Sequence
  Dengue2-3
agucaggucggauuaagccauaguacggaaaaaacuaugcuaccugugagccccguccaaggacg

SEQ ID NO: 38 - RNA, 65 nt, Artificial Sequence
  Dengue3-3
ugucaggccaccuuaagccacaguacggaagaagcugugcugccugugagccccguccaaggacg

SEQ ID NO: 39 - RNA, 69 nt, Artificial Sequence
  Kokobera-3
agucaggccugaaaagccaccugauccggugaaggugcugccugcauccggccuggagugaugcuccag

SEQ ID NO: 40 - RNA, 71 nt, Artificial Sequence
  Zika-3.1
ugucaggccugcuagucagccacaguuuggggaaagcugugcagccuguaacccccccaggagaagcuggg

SEQ ID NO: 41 - RNA, 74 nt, Artificial Sequence
  St. Louis encephalitis-3
agucaggccaaucaguuuugccaccggaugucagguaaacggugcugucuguaaccuggccccaggcgacuggg

SEQ ID NO: 42 - RNA, 75 nt, Artificial Sequence
  Japanese encephalitis-3
agucaggccagcaaaagcugccaccggauacuggguagacggugcugucugcgucucagucccaggaggacuggg

SEQ ID NO: 43 - RNA, 75 nt, Artificial Sequence; identical to SEQ ID NO: 36
  Ntaya-3

SEQ ID NO: 44 - RNA, 75 nt, Artificial Sequence
  Usutu-3
agucaggccagggcaaccugccaccggaaguugaguagacggugcugccugcgacucaaccccaggcggacuggg

SEQ ID NO: 45 - RNA, 76 nt, Artificial Sequence
  Murray Valley encephalitis-3.1
agucaggccagccgguuaggcugccaccgaagguugguagacggugcugccugcgaccaaccccaggaggacuggg

SEQ ID NO: 46 - RNA, 76 nt, Artificial Sequence
  West Nile-3.1
agucaggccagauuaaugcugccaccggaaguugaguagacggugcugccugcggcucaaccccaggaggacuggg

SEQ ID NO: 47 - RNA, 61 nt, Artificial Sequence
  Dengue1-4
agucaggccgaaagccacgguucgagcaagccgugcugccuguagcuccaucguggggaug

SEQ ID NO: 48 - RNA, 67 nt, Artificial Sequence
  Dengue2-4
agucaggccaucauaaaugccauagcuugaguaaacuaugcagccuguagcuccaccugagaaggug

SEQ ID NO: 49 - RNA, 62 nt, Artificial Sequence
  Dengue3-4
agucaggccccaaagccacgguuugagcaaaccgugcugccuguagcuccgucguggggacg

SEQ ID NO: 50 - RNA, 71 nt, Artificial Sequence
  Kokobera-4
agucagauccgaaaggccaccaguuuggugcagaacuggugcuaucugugaacacucccaggaggacuggg

SEQ ID NO: 51 - RNA, 68 nt, Artificial Sequence
  Zika-4
agucaggccgagaacgccauggcacggaagaagccaugcugccugugagccccucagaggacacugag

SEQ ID NO: 52 - RNA, 69 nt, Artificial Sequence
  St. Louis encephalitis -4
agucagaccagaaaugccaccugaaagcaugcuaaaggugcugucuguacaugccccaggaggacuggg

SEQ ID NO: 53 - RNA, 66 nt, Artificial Sequence
  Japanese encephalitis-4
cgucaggccacaaauuugugccaccccgcuagggggugcggccugcgcagccccaggaggacuggg

SEQ ID NO: 54 - RNA, 72 nt, Artificial Sequence
  Ntaya-4
agucaggccguagguuuuacgccacuagcaugcagugcugccugagacaagacaugccccaggaggacuggg

SEQ ID NO: 55 - RNA, 63 nt, Artificial Sequence
  Usutu-4
ugucaggccgcaaagcgccacuucgccaaggagugcagccuguacggccccaggaggacuggg

SEQ ID NO: 56 - RNA, 64 nt, Artificial Sequence
  Murray Valley encephalitis-4.1
ugucagaucgcgaaagcgccacuucgccgaggagugcaaucugugaggccccaggaggacuggg

SEQ ID NO: 57 - RNA, 70 nt, Artificial Sequence
  West Nile-4
ugucagaccacacuuuaaugugccacucugcggagagugcagucugcgauagugccccagguggacuggg

SEQ ID NO: 58 - RNA, 68 nt, Artificial Sequence
  Dengue4-2.1
agucaggccacuugugccacgguuugagcaaaccgugcugccuguagcuccgccaauaaugggaggcg

SEQ ID NO: 59 - RNA, 72 nt, Artificial Sequence
  Wesselbron-2
agucagcccaucauuugaugccauggcuaagcugugaggccaugcuggcugggacagccgcgaccacccgcg

SEQ ID NO: 60 - RNA, 73 nt, Artificial Sequence
  Ilheus-2
ugucaggccauggaaacaugccacccaaagcuuguagagggugcagccugcgccaagccccaggaggacuggg

SEQ ID NO: 61 - RNA, 73 nt, Artificial Sequence
  Sepik-2
ugucagcccgucauaaugacgccauggcuaagcugugaggccaugcuggcugggauagccgcgaccacccgcg

SEQ ID NO: 62 - RNA, 75 nt, Artificial Sequence
  Bussuquara-2
agucaggccagaaaugccaccggauaaagguagacggugcugccugcaaccuuucugcggaaggaauaaccgcag

SEQ ID NO: 63 - RNA, 75 nt, Artificial Sequence
  Tembusu-2
agucaggccagggaaucccugccaccggauguuggaugacggugcugucugcguuccaaccccaggaggacuggg

SEQ ID NO: 64 - RNA, 77 nt, Artificial Sequence
  Chaoyang-2
agucaggccuaaaugccaccggaugauaguagacggugcugccugcagcuaucacauauaaacuggcgccuuauaug

SEQ ID NO: 65 - RNA, 80 nt, Artificial Sequence
  Kedougou-2
cgucaggccacucgugagagugccacaguacgguaaagacugugcggccugcgagcccccugggacuaucgguauccagg

SEQ ID NO: 66 - RNA, 83 nt, Artificial Sequence
  Yokose-2
ugucaggccaagauugagaaaaucuugccacagcuuggcagacugugcagccugcagcccuagagggagacugaccaacuccc

SEQ ID NO: 67 - RNA, 85 nt, Artificial Sequence
  Dongang-2
agucaggccucacgaaugugagccaccggaugggacuagacggugcugccugcgcguccccccacgcaaucaagcuagaugguug

SEQ ID NO: 68 - RNA, 93 nt, Artificial Sequence
  Yellow Fever-2
ugucagcccagaaccccacacgaguuuugccacugcuaagcugugaggcagugcaggcugggacagccgaccuccagguugcgaaaaaccugg

SEQ ID NO: 69 - RNA, 79 nt, Artificial Sequence
  Tamana bat virus-1
gaaaggcaagguacggauuagccguaggggcuugagaacccccccuccccacucauuuuauuuccucuaugaggaaggu

SEQ ID NO: 70 - RNA, 76 nt, Artificial Sequence
  Tamana bat virus-2
uuugggcaaggugcagguuagcugcaggggcuugaaaaacccccccccccauucaagacuuuuagugcauuaguuu

SEQ ID NO: 71 - RNA, 81 nt, Artificial Sequence
  Culex flavivirus-1
accccguaaggaaggacaaggcuguccuugaguacuaacgacacuccggccccaguucccagagccaggguuuuagcucca

SEQ ID NO: 72 - RNA, 81 nt, Artificial Sequence
  Culex flavivirus-2
cgcgcgcaaggaaggacauggcuguccuuggguacgaacgacaccccgcccccaguucucaagguuagaguuauaaccuca

SEQ ID NO: 73 - RNA, 75 nt, Artificial Sequence
  Culex flavivirus-3
ccaucgcaagggaggauuuuccucggguacugaccauaccccgaccccaguccgauaggucauggaaugacccca

SEQ ID NO: 74 - RNA, 79 nt, Artificial Sequence
  Culex flavivirus-4
cucccguaaggaaagcgcaagcuuugagcauugacaacgcuccggccccagucccccagguuaugggagaauaacccca

SEQ ID NO: 75 - RNA, 104 nt, Artificial Sequence
  Rodent pestivirus
acacggcaaggugcuagaagcugaaaccgacucggagcucuagcagggggacuggcgacccucccgccccagcuggccucuggcagaaacgacucgugccauug

SEQ ID NO: 76 - RNA, 65 nt, Artificial Sequence
  Simian pegivirus
cuccaggcagcagcagacgcaagucuggggggugcugucugcgucucagucccaggaggacuggg

SEQ ID NO: 77 - RNA, 63 nt, Artificial Sequence
  GB virus B
cagcggcaacaggggagaccccgggcuuagugcugucugcgcuccaaccccaggcggacuggg

SEQ ID NO: 78 - RNA, 78 nt, Artificial Sequence
  Murray Valley encephalitis -3.2
uagucaggccagccgguuaggcugccaccgaagguugguagacggugcugccugcgaccaaccccaggaggacugggu

SEQ ID NO: 79 - RNA, 77 nt, Artificial Sequence
  West Nile-3.2
agucaggccagauuaaugcugccaccggaaguugaguagacggugcugccugcggcucaaccccaggaggacugggu

SEQ ID NO: 80 - RNA, 81 nt, Artificial Sequence
  Zika-3.2
ugucaggccugcuagucagccacaguuuggggaaagcugugcagccuguaacccccccaggagaagcugggaaaccaagcu

SEQ ID NO: 81 - RNA, 69 nt, Artificial Sequence
  Dengue4-2.2
agucaggccacuugugccacgguuugagcaaaccgugcugccuguagcuccgccaauaaugggaggcgu

SEQ ID NO: 82 - RNA, 108 nt, Artificial Sequence
  Dengue1-5
agucaggccggauuaagccauagcacgguaagagcuaugcugccugugagccuaaaaugaagucaggccgaaagccacgguucgagcaagccgugcugccuguagcuc

SEQ ID NO: 83 - RNA, 105 nt, Artificial Sequence
  Dengue2-5
agucaggucggauuaagccauaguacggaaaaaacuaugcuaccugugagccagucaggccaucauaaaugccauagcuugaguaaacuaugcagccuguagcuc

SEQ ID NO: 84 - RNA, 112 nt, Artificial Sequence
  Dengue3-5
ugucaggccaccuuaagccacaguacggaagaagcugugcugccugugagccuuaaaagaagaagucaggccccaaagccacgguuugagcaaaccgugcugccuguagcuc

SEQ ID NO: 85 - RNA, 99 nt, Artificial Sequence
  Kokobera-5
agucaggccugaaaagccaccugauccggugaaggugcugccugcauccgguagcaagucagauccgaaaggccaccaguuuggugcagaacuggugcu

SEQ ID NO: 86 - RNA, 119 nt, Artificial Sequence
  Zika-5
ugucaggccugcuagucagccacaguuuggggaaagcugugcagccuguaaccccaaaccaagcucauagucaggccgagaacgccauggcacggaagaagccaugcugccugugagcc

SEQ ID NO: 87 - RNA, 157 nt, Artificial Sequence
  St. Louis encephalitis-5
agucaggccaaucaguuuugccaccggaugucagguaaacggugcugucuguaaccuggcuuaucaaagccaacccggcugggugcaaagccccucauuccgaagucagaccagaaaugccaccugaaagcaugcuaaaggugcugucuguacaugc

SEQ ID NO: 88 - RNA, 134 nt, Artificial Sequence
  Dengue1-6
agucaggccggauuaagccauagcacgguaagagcuaugcugccugugagccccguccaaggacguaaaaugaagucaggccgaaagccacgguucgagcaagccgugcugccuguagcuccaucguggggaug

SEQ ID NO: 89 - RNA, 140 nt, Artificial Sequence
  Dengue2-6
agucaggucggauuaagccauaguacggaaaaaacuaugcuaccugugagccccguccaaggacguuaaaagaagucaggccaucauaaaugccauagcuugaguaaacuaugcagccuguagcuccaccugagaaggug

SEQ ID NO: 90 - RNA, 138 nt, Artificial Sequence
  Dengue3-6
ugucaggccaccuuaagccacaguacggaagaagcugugcugccugugagccccguccaaggacguuaaaagaagaagucaggccccaaagccacgguuugagcaaaccgugcugccuguagcuccgucguggggacg

SEQ ID NO: 91 - RNA, 233 nt, Artificial Sequence
  Kokobera-6
agucaggccugaaaagccaccugauccggugaaggugcugccugcauccggccuggagugaugcuccagugucguggaacaacaaccgauggagccaagcccggaggggauccggcccccgacuuccggagguugccacaccuuguaaauauguacauacagagucagauccgaaaggccaccaguuuggugcagaacuggugcuaucugugaacacucccaggaggacuggg

SEQ ID NO: 92 - RNA, 152 nt, Artificial Sequence
  Zika-6.1
ugucaggccugcuagucagccacaguuuggggaaagcugugcagccuguaacccccccaggagaagcugggaaaccaagcucauagucaggccgagaacgccauggcacggaagaagccaugcugccugugagccccucagaggacacugag

SEQ ID NO: 93 - RNA, 227 nt, Artificial Sequence
  St. Louis encephalitis-6
agucaggccaaucaguuuugccaccggaugucagguaaacggugcugucuguaaccuggccccaggcgacuggguuaucaaagccaacccggcugggugcaaagccccucauuccgacucgggagggucccuggcacguaggcuggagaggacgcacaagucagaccagaaaugccaccugaaagcaugcuaaaggugcugucuguacaugccccaggaggacuggg

SEQ ID NO: 94 - RNA, 226 nt, Artificial Sequence
  Japanese encephalitis-5
agucaggccagcaaaagcugccaccggauacuggguagacggugcugucugcgucucagucccaggaggacuggguuaacaaaucugacaacagaaagugagaaagcccucagaaccgucucggaaguaggucccugcucacuggaaguugaaagaccaacgucaggccacaaauuugugccaccccgcuagggggugcggccugcgcagccccaggaggacuggg

SEQ ID NO: 95 - RNA, 231 nt, Artificial Sequence
  Ntaya-5
agucaggccaggcaaugccugccaccggaaguuggaugacggugcugucugcgcuccaaccccaggcggacuggguuaacaaaguggugauccaggagacgcaaagcguucacuaccguuucggagaacucccuggcguucgaauggaacacugcccaaagucaggccguagguuuuacgccacuagcaugcagugcugccugagacaagacaugccccaggaggacuggg

SEQ ID NO: 96 - RNA, 222 nt, Artificial Sequence
  Usutu-5
agucaggccagggcaaccugccaccggaaguugaguagacggugcugccugcgacucaaccccaggcggacuggguuaacaaagcugaccgcugaugaugggaaagccccucagaaccguuucggagagggacccugccuauuggaagcguccagcccgugucaggccgcaaagcgccacuucgccaaggagugcagccuguacggccccaggaggacuggg

SEQ ID NO: 97 - RNA, 226 nt, Artificial Sequence
  Murray Valley encephalitis-5
agucaggccagccgguuaggcugccaccgaagguugguagacggugcugccugcgaccaaccccaggaggacuggguuaccaaagcugauucuccacgguuggaaagccucccagaaccgucucggaagaggagucccugccaacaauggagaugaagcccgugucagaucgcgaaagcgccacuucgccgaggagugcaaucugugaggccccaggaggacuggg

SEQ ID NO: 98 - RNA, 230 nt, Artificial Sequence
  West Nile-5.1
agucaggccagauuaaugcugccaccggaaguugaguagacggugcugccugcggcucaaccccaggaggacugggugaccaaagcugcgaggugauccacguaagcccucagaaccgucucggaaggaggaccccacgugcuuuagccucaaagcccagugucagaccacacuuuaaugugccacucugcggagagugcagucugcgauagugccccagguggacuggg

SEQ ID NO: 99 - RNA, 231 nt, Artificial Sequence
  West Nile-5.2
agucaggccagauuaaugcugccaccggaaguugaguagacggugcugccugcggcucaaccccaggaggacugggugaccaaagcugcgaggugauccacguaagcccucagaaccgucucggaaggaggaccccacgugcuuuagccucaaagcccagugucagaccacacuuuaaugugccacucugcggagagugcagucugcgauagugccccagguggacugggu

SEQ ID NO: 100 - RNA, 129 nt, Artificial Sequence
  Zika-6.2
ugucaggccugcuagucagccacaguuuggggaaagcugugcagccuguaacccccccaggagaagcugggaaaccaagcucauagucaggccgagaacgccauggcacggaagaagccaugcugccug

SEQ ID NO: 101 - RNA, 89 nt, Artificial Sequence
  Tick-borne encephalitis virus-1
agaucauggaaugaugcggcagcgcgcgagagcgacggggaaguggucgcacccgacgcaccauccaugaagcaauacuucgugagacc

SEQ ID NO: 102 - RNA, 94 nt, Artificial Sequence
  Tick-borne encephalitis virus-2
cccccagagugcauuacggcagcacgccagugagaguggcgacgggaaaauggucgaucccgacguagggcacucugaaaaauuuugugagacc

SEQ ID NO: 103 - RNA, 90 nt, Artificial Sequence
  Montana myotis leukoencephalitis virus
ggaaaaaaugcgagugagggcaacucugggauuagcucaaugggugugacgacccuacccuuccgcauuuguaaauaauugagccaguca

SEQ ID NO: 104 - RNA, 89 nt, Artificial Sequence
  Modoc virus
augaaagaguguugagggcaaccagugggcuagccacauggguaugacgcacccacccucugcauucuuguaaauacuuuggccaguca

SEQ ID NO: 105 - RNA, 96 nt, Artificial Sequence
  Apoi virus
auaagcgccuggagagcgaccuuggacuguggggguccaaagggcuuugcgacccuucucucuuugagcgcuuugggacaacuauuuggccagcau

SEQ ID NO: 106 - RNA, 98 nt, Artificial Sequence
  Opium poppy mosaic virus
gaauugccuccaccaguaacuaaacccaaccacagccaagcauuaaguugcaagcguuggaguggcaggcuuaacguccgacaguacgacaacugcgg

SEQ ID NO: 107 - RNA, 97 nt, Artificial Sequence
  Maize chlorotic mottle virus
guuccaggcccagggcuggcaaaucauugagcacaaggugagccggcaugagguugcaagaccggaacaaccaguccuucuggcagaguccugccaa

SEQ ID NO: 108 - RNA, 83 nt, Artificial Sequence
  Potato leafroll virus
gccaccacaaaagaacacugaaggagcucacuaaaacuagccaagcauacacgaguugcaagcauuggaaguucaagccucgu

SEQ ID NO: 109 - RNA, 86 nt, Artificial Sequence
  Maize yellow dwarf virus-RMV
guccagaaacaaaaaguuuaaaacagaagcucucaagucagccaggcaaauucgaguugcaagcacuggaugaccuagucucgaua

SEQ ID NO: 110 - RNA, 85 nt, Artificial Sequence
  Hubei polero-like virus1
gccacaaaacgaauaaaggaagaacgcacgagagucagccaaacaaacacaaguugcaaguguuggagacucauucuagucuugu

SEQ ID NO: 111 - DNA, 44 bp, Artificial Sequence
  Red clover necrotic mosaic virus-1
gcgtagcctccacccgagttgcaagaggggaacgcgcagtctcg

SEQ ID NO: 112 - DNA, 44 bp, Artificial Sequence
  Sweet clover necrotic mosaic virus
gcgtaacctccatccgagttgcaagagagggaaacgcagtctcg

SEQ ID NO: 113 - DNA, 46 bp, Artificial Sequence
  Carnation ringspot virus
ccgtagccgccaacaaagttgcaagagcgggcgttgctagcctttg

SEQ ID NO: 114 - DNA, 130 bp, Artificial Sequence
  Red clover necrotic mosaic virus-2
gcgtagcctccacccgagttgcaagaggggaacgcgcagtctcgccgaccctgttggcaaacagtaaaattgcaaaaaatagagtgctaggagtagttcccgtacccgcgggagcaagaccctactacag

SEQ ID NO: 115 - RNA, 129 nt, Artificial Sequence
  cgRNA Q
gagucgcguguagcgaagcaguuuuagagcuagaaauagcaaguuaaaauaaggcuaguccgaucuuugcgcguuaguuucguucguauuucugucauguuugcgcggcaccgagucggugcuuuuuuu

SEQ ID NO: 116 - RNA, 48 nt, Artificial Sequence
  Trigger XQ
aaacaugacagaaauacgaacgaaacuaacgcgcaaagaucuuuuuuu

SEQ ID NO: 117 - RNA, 126 nt, Artificial Sequence
  MVE
uagucaggccagccgguuaggcugccaccgaagguugguagacggugcugccugcgaccaaccccaggaggacuggguaaacaugacagaaauacgaacgaaacuaacgcgcaaagaucuuuuuuu

SEQ ID NO: 118 - RNA, 125 nt, Artificial Sequence
  WNV
agucaggccagauuaaugcugccaccggaaguugaguagacggugcugccugcggcucaaccccaggaggacuggguaaacaugacagaaauacgaacgaaacuaacgcgcaaagaucuuuuuuu

SEQ ID NO: 119 - DNA, 129 bp, Artificial Sequence
  Zika
ugucaggccugcuagucagccacaguuuggggaaagcugugcagccuguaacccccccaggagaagcugggaaaccaagctaaacaugacagaaauacgaacgaaacuaacgcgcaaagaucuuuuuuu

SEQ ID NO: 120 - RNA, 117 nt, Artificial Sequence
  Dengue
agucaggccacuugugccacgguuugagcaaaccgugcugccuguagcuccgccaauaaugggaggcguaaacaugacagaaauacgaacgaaacuaacgcgcaaagaucuuuuuuu

SEQ ID NO: 121 - RNA, 129 nt, Artificial Sequence; identical to SEQ ID NO: 115
  cgRNA Q

SEQ ID NO: 122 - RNA, 129 nt, Artificial Sequence
  cgRNA R
gagucgcguguagcgaagcaguuuuagagcuagaaauagcaaguuaaaauaaggcuaguccguaucgccggguucaagcagauguggcauuucaguguaguucccgggcaccgagucggugcuuuuuuu

SEQ ID NO: 123 - RNA, 129 nt, Artificial Sequence
  cgRNA S
gagucgcguguagcgaagcaguuuuagagcuagaaauagcaaguuaaaauaaggcuaguccguccauucggguuuacuauuacaaucuuacguguucucauucccgggcaccgagucggugcuuuuuuu

SEQ ID NO: 124 - RNA, 129 nt, Artificial Sequence
  cgRNA T
gagucgcguguagcgaagcaguuuuagagcuagaaauagcaaguuaaaauaaggcuaguccggauaaagggaaagaugaagugaugugaagauagaguuggaucccggcaccgagucggugcuuuuuuu

SEQ ID NO: 125 - RNA, 48 nt, Artificial Sequence; identical to SEQ ID NO: 116
  Trigger XQ

SEQ ID NO: 126 - RNA, 48 nt, Artificial Sequence
  Trigger XR
aacuacacugaaaugccacaucugcuugaacccggcgauacuuuuuuu

SEQ ID NO: 127 - RNA, 48 nt, Artificial Sequence
  Trigger XS
aaugagaacacguaagauuguaauaguaaacccgaauggacuuuuuuu

SEQ ID NO: 128 - RNA, 48 nt, Artificial Sequence
  Trigger XT
uccaacucuaucuucacaucacuucaucuuucccuuuauccuuuuuuu

SEQ ID NO: 129 - RNA, 117 nt, Artificial Sequence; identical to SEQ ID NO: 120
  Trigger XQ

SEQ ID NO: 130 - RNA, 117 nt, Artificial Sequence
  Trigger XR
agucaggccacuugugccacgguuugagcaaaccgugcugccuguagcuccgccaauaaugggaggcguaacuacacugaaaugccacaucugcuugaacccggcgauacuuuuuuu

SEQ ID NO: 131 - RNA, 117 nt, Artificial Sequence
  Trigger XS
agucaggccacuugugccacgguuugagcaaaccgugcugccuguagcuccgccaauaaugggaggcguaaugagaacacguaagauuguaauaguaaacccgaauggacuuuuuuu

SEQ ID NO: 132 - RNA, 117 nt, Artificial Sequence
  Trigger XT
agucaggccacuugugccacgguuugagcaaaccgugcugccuguagcuccgccaauaaugggaggcguuccaacucuaucuucacaucacuucaucuuucccuuuauccuuuuuuu

SEQ ID NO: 133 - RNA, 129 nt, Artificial Sequence; identical to SEQ ID NO: 115
  cgRNA

SEQ ID NO: 134 - RNA, 48 nt, Artificial Sequence; identical to SEQ ID NO: 116
  Trigger

SEQ ID NO: 135 - RNA, 110 nt, Artificial Sequence
  MVE-1 PEL + trigger
uagucaggccagccgguuaggcugccaccgaagguugguagacggugcugccugcgaccaacaaacaugacagaaauacgaacgaaacuaacgcgcaaagaucuuuuuuu

SEQ ID NO: 136 - RNA, 126 nt, Artificial Sequence; identical to SEQ ID NO: 117
  MVE-2 PEL + trigger

SEQ ID NO: 137 - RNA, 109 nt, Artificial Sequence
  WNV-1 PEL + trigger
agucaggccagauuaaugcugccaccggaaguugaguagacggugcugccugcggcucaacaaacaugacagaaauacgaacgaaacuaacgcgcaaagaucuuuuuuu

SEQ ID NO: 138 - RNA, 125 nt, Artificial Sequence; identical to SEQ ID NO: 118
  WNV-2 PEL + trigger

SEQ ID NO: 139 - RNA, 279 nt, Artificial Sequence
  WNV-3 PEL + trigger
agucaggccagauuaaugcugccaccggaaguugaguagacggugcugccugcggcucaaccccaggaggacugggugaccaaagcugcgaggugauccacguaagcccucagaaccgucucggaaggaggaccccacgugcuuuagccucaaagcccagugucagaccacacuuuaaugugccacucugcggagagugcagucugcgauagugccccagguggacuggguaaacaugacagaaauacgaacgaaacuaacgcgcaaagaucuuuuuuu

SEQ ID NO: 140 - RNA, 112 nt, Artificial Sequence
  Zika-1 PEL + trigger
ugucaggccugcuagucagccacaguuuggggaaagcugugcagccuguaacccccccaggagaaaacaugacagaaauacgaacgaaacuaacgcgcaaagaucuuuuuuu

SEQ ID NO: 141 - RNA, 129 nt, Artificial Sequence
  Zika-2 PEL + trigger
ugucaggccugcuagucagccacaguuuggggaaagcugugcagccuguaacccccccaggagaagcugggaaaccaagcuaaacaugacagaaauacgaacgaaacuaacgcgcaaagaucuuuuuuu

SEQ ID NO: 142 - RNA, 177 nt, Artificial Sequence
  Zika-3 PEL + trigger
ugucaggccugcuagucagccacaguuuggggaaagcugugcagccuguaacccccccaggagaagcugggaaaccaagcucauagucaggccgagaacgccauggcacggaagaagccaugcugccugaaacaugacagaaauacgaacgaaacuaacgcgcaaagaucuuuuuuu

SEQ ID NO: 143 - RNA, 117 nt, Artificial Sequence; identical to SEQ ID NO: 120
  Dengue PEL + trigger

SEQ ID NO: 144 - RNA, 48 nt, Artificial Sequence; identical to SEQ ID NO: 116
  RNA XQ

SEQ ID NO: 145 - RNA, 48 nt, Artificial Sequence; identical to SEQ ID NO: 128
  RNA XT

SEQ ID NO: 146 - RNA, 117 nt, Artificial Sequence; identical to SEQ ID NO: 120
  PEL + RNA XQ

SEQ ID NO: 147 - RNA, 117 nt, Artificial Sequence; identical to SEQ ID NO: 132
  PEL + RNA XT

SEQ ID NO: 148 - RNA, 48 nt, Artificial Sequence; identical to SEQ ID NO: 116
  RNA XQ - Without PEL

SEQ ID NO: 149 - RNA, 117 nt, Artificial Sequence; identical to SEQ ID NO: 120
  RNA XQ - With 5′ PEL

SEQ ID NO: 150 - RNA, 188 nt, Artificial Sequence
  5′ RNA spacer + MVE PEL + trigger
cucgaggugagcaagggcgaggagcuguucaccgggguuggugcccauccugguggacuugauagucaggccagccgguuaggcugccaccgaagguugguagacggugcugccugcgaccaaccccaggaggacuggguaaacaugacagaaauacgaacgaaacuaacgcgcaaagaucuuuuuuu

SEQ ID NO: 151 - RNA, 48 nt, Artificial Sequence; identical to SEQ ID NO: 116
  Trigger

SEQ ID NO: 152 - RNA, 110 nt, Artificial Sequence; identical to SEQ ID NO: 135
  MVE-1 PEL + trigger

SEQ ID NO: 153 - RNA, 126 nt, Artificial Sequence; identical to SEQ ID NO: 117
  MVE-2 PEL + trigger

SEQ ID NO: 154 - RNA, 117 nt, Artificial Sequence; identical to SEQ ID NO: 120
  Dengue PEL + trigger

SEQ ID NO: 155 - RNA, 142 nt, Artificial Sequence
  YF  PEL + trigger
ugucagcccagaaccccacacgaguuuugccacugcuaagcugugaggcagugcaggcugggacagccgaccuccagguugcgaaaaaccugguaaacaugacagaaauacgaacgaaacuaacgcgcaaagaucuuuuuuu

SEQ ID NO: 156 - DNA, 38 bp, Artificial Sequence
  Riboswitch-1 (computationally designed)
acgcggttctatctagttacgcgttaaaccaactagaa

SEQ ID NO: 157 - DNA, 37 bp, Artificial Sequence
  Riboswitch-2 (computationally designed)
cgcggttctatctagttacgcgttaaaccaactagaa

SEQ ID NO: 158 - DNA, 45 bp, Artificial Sequence
  Riboswitch-3 (computationally designed)
acgcggttctatctagttacgcgttaaaccaactagaaggcggtt

SEQ ID NO: 159 - DNA, 38 bp, Artificial Sequence
  Riboswitch-4 (computationally designed)
acgcggttctactaggttacgcgttaaacaacctagaa

SEQ ID NO: 160 - RNA, 55 nt, Artificial Sequence
  Reverse Zika-1.2
gggguuucuggcugcucugcuuuccccuuucuguggcugucuugcuggccugucu

SEQ ID NO: 161 - RNA, 50 nt, Artificial Sequence
  Reverse Dengue4-1
gugcuucuggcugcucgguuugcucuuuccguggcucuuguggccugucu

SEQ ID NO: 162 - RNA, 90 nt, Artificial Sequence
  Reverse montana myotis leukoencephalitis virus
ugacuggcucaauuauuuacaaaugcggaaggguagggucgucacacccauugagcuaaucccagaguugcccucacucgcauuuuuucc

SEQ ID NO: 163 - RNA, 89 nt, Artificial Sequence
  Reverse modoc virus
ugacuggccaaaguauuuacaagaaugcagagggugggugcgucauacccauguggcuagcccacugguugcccucaacacucuuucau

SEQ ID NO: 164 - RNA, 98 nt, Artificial Sequence
  Reverse opium poppy mosaic virus
ccgcaguugucguacugucggacguuaagccugccacuccaacgcuugcaacuuaaugcuuggcugugguuggguuuaguuacugguggaggcaauuc

SEQ ID NO: 165 - RNA, 44 nt, Artificial Sequence
  Reverse sweet clover necrotic mosaic virus
cgagacugcguuucccucucuugcaacucggauggagguuacgc

SEQ ID NO: 166 - RNA, 38 nt, Artificial Sequence
  Reverse riboswitch-1
uucuaguugguuuaacgcguaacuagauagaaccgcgu

SEQ ID NO: 167 - RNA, 37 nt, Artificial Sequence
  Reverse riboswitch-2
uucuaguugguuuaacgcguaacuagauagaaccgcg

SEQ ID NO: 168 - RNA, 129 nt, Artificial Sequence; identical to SEQ ID NO: 115
  cgRNA

SEQ ID NO: 169 - RNA, 48 nt, Artificial Sequence; identical to SEQ ID NO: 116
  Trigger

SEQ ID NO: 170 - RNA, 117 nt, Artificial Sequence; identical to SEQ ID NO: 120
  Dengue4-ph PEL + trigger

SEQ ID NO: 171 - RNA, 98 nt, Artificial Sequence
  Dengue4-p PEL + trigger
agucaggccacuugugccacgguuugagcaaaccgugcugccuguagcucaaacaugacagaaauacgaacgaaacuaacgcgcaaagaucuuuuuuu

SEQ ID NO: 172 - RNA, 137 nt, Artificial Sequence
  MODV PEL + trigger
augaaagaguguugagggcaaccagugggcuagccacauggguaugacgcacccacccucugcauucuuguaaauacuuuggccagucaaaacaugacagaaauacgaacgaaacuaacgcgcaaagaucuuuuuuu

SEQ ID NO: 173 - RNA, 119 nt, Artificial Sequence
  Zika-1-ph PEL + trigger
ugucaggccugcuagucagccacaguuuggggaaagcugugcagccuguaacccccccaggagaagcugggaaacaugacagaaauacgaacgaaacuaacgcgcaaagaucuuuuuuu

SEQ ID NO: 174 - RNA, 116 nt, Artificial Sequence
  Zika-2-ph PEL + trigger
agucaggccgagaacgccauggcacggaagaagccaugcugccugugagccccucagaggacacugagaaacaugacagaaauacgaacgaaacuaacgcgcaaagaucuuuuuuu

SEQ ID NO: 175 - RNA, 200 nt, Artificial Sequence
  Zika-ph+ph PEL + trigger
ugucaggccugcuagucagccacaguuuggggaaagcugugcagccuguaacccccccaggagaagcugggaaaccaagcucauagucaggccgagaacgccauggcacggaagaagccaugcugccugugagccccucagaggacacugagaaacaugacagaaauacgaacgaaacuaacgcgcaaagaucuuuuuuu

SEQ ID NO: 176 - RNA, 125 nt, Artificial Sequence; identical to SEQ ID NO: 118
  WNV-ph  PEL + trigger

SEQ ID NO: 177 - RNA, 138 nt, Artificial Sequence
  MMLV PEL + trigger
ggaaaaaaugcgagugagggcaacucugggauuagcucaaugggugugacgacccuacccuuccgcauuuguaaauaauugagccagucaaaacaugacagaaauacgaacgaaacuaacgcgcaaagaucuuuuuuu

SEQ ID NO: 178 - RNA, 120 nt, Artificial Sequence
  Wesselbron PEL + trigger
agucagcccaucauuugaugccauggcuaagcugugaggccaugcuggcugggacagccgcgaccacccgcgaaacaugacagaaauacgaacgaaacuaacgcgcaaagaucuuuuuuu

SEQ ID NO: 179 - RNA, 125 nt, Artificial Sequence
  Chaoyang PEL + trigger
agucaggccuaaaugccaccggaugauaguagacggugcugccugcagcuaucacauauaaacuggcgccuuauaugaaacaugacagaaauacgaacgaaacuaacgcgcaaagaucuuuuuuu

SEQ ID NO: 180 - RNA, 103 nt, Artificial Sequence
  WNV-2  PEL + trigger
ugucagaccacacuuuaaugugccacucugcggagagugcagucugcgauagugcaaacaugacagaaauacgaacgaaacuaacgcgcaaagaucuuuuuuu

SEQ ID NO: 181 - RNA, 118 nt, Artificial Sequence
  WNV-4  PEL + trigger
ugucagaccacacuuuaaugugccacucugcggagagugcagucugcgauagugccccagguggacugggaaacaugacagaaauacgaacgaaacuaacgcgcaaagaucuuuuuuu

SEQ ID NO: 182 - RNA, 110 nt, Artificial Sequence
  CFAV-2-ph PEL + trigger
agcagggcacauuagugucgggcgugacgcacccgcuccccucaguccccugugcaacagggaaacaugacagaaauacgaacgaaacuaacgcgcaaagaucuuuuuuu

SEQ ID NO: 183 - RNA, 112 nt, Artificial Sequence; identical to SEQ ID NO: 140
  Zika-1-p PEL + trigger

SEQ ID NO: 184 - RNA, 92 nt, Artificial Sequence
  RCNMV PEL + trigger
gcguagccuccacccgaguugcaagaggggaacgcgcagucucgaaacaugacagaaauacgaacgaaacuaacgcgcaaagaucuuuuuuu

SEQ ID NO: 185 - RNA, 92 nt, Artificial Sequence
  SCNMV PEL + trigger
gcguaaccuccauccgaguugcaagagagggaaacgcagucucgaaacaugacagaaauacgaacgaaacuaacgcgcaaagaucuuuuuuu

SEQ ID NO: 186 - RNA, 86 nt, Artificial Sequence
  Rbsw-1 PEL + trigger
acgcgguucuaucuaguuacgcguuaaaccaacuagaaaaacaugacagaaauacgaacgaaacuaacgcgcaaagaucuuuuuuu

SEQ ID NO: 187 - RNA, 85 nt, Artificial Sequence
  Rbsw-2 PEL + trigger
cgcgguucuaucuaguuacgcguuaaaccaacuagaaaaacaugacagaaauacgaacgaaacuaacgcgcaaagaucuuuuuuu

SEQ ID NO: 188 - RNA, 93 nt, Artificial Sequence
  Rbsw-3 PEL + trigger
acgcgguucuaucuaguuacgcguuaaaccaacuagaaggcgguuaaacaugacagaaauacgaacgaaacuaacgcgcaaagaucuuuuuuu

SEQ ID NO: 189 - RNA, 86 nt, Artificial Sequence
  Rbsw-4 PEL + trigger
acgcgguucuacuagguuacgcguuaaacaaccuagaaaaacaugacagaaauacgaacgaaacuaacgcgcaaagaucuuuuuuu

SEQ ID NO: 190 - DNA, 106 bp, Artificial Sequence
  RNA XQ
aaacatgacagaaatacgaacgaaactaacgcgcaaagatcgcaattaaacaatcctaacgtagaacaggatcacctggaagtgttaccgtcaatcgcctcaccta

SEQ ID NO: 191 - DNA, 222 bp, Artificial Sequence
  5′ RNA spacer + Y.fever-ph PEL +  RNA XQ
cuuguggaaaggacgaaacaccugucagcccagaaccccacacgaguuuugccacugcuaagcugugaggcagugcaggcugggacagccgaccuccagguugcgaaaaaccugguaaacatgacagaaatacgaacgaaactaacgcgcaaagatcgcaattaaacaatcctaacgtagaacaggatcacctggaagtgttaccgtcaatcgcctcaccta

SEQ ID NO: 192 - DNA, 197 bp, Artificial Sequence
  5′ RNA spacer + Dengue4-ph PEL + RNA XQ
cuuguggaaaggacgaaacaccagucaggccacuugugccacgguuugagcaaaccgugcugccuguagcuccgccaauaaugggaggcguaaacatgacagaaatacgaacgaaactaacgcgcaaagatcgcaattaaacaatcctaacgtagaacaggatcacctggaagtgttaccgtcaatcgcctcaccta

SEQ ID NO: 193 - DNA, 257 bp, Artificial Sequence
  5′ RNA spacer + ZIKA-ph+ph PEL + RNA XQ
cuuguggaaaggacgaaacaccugucaggccugcuagucagccacaguuuggggaaagcugugcagccuguaacccccccaggagaagcugggaaaccaagcucauagucaggccgagaacgccauggcacggaagaagccaugcugccugaaacatgacagaaatacgaacgaaactaacgcgcaaagatcgcaattaaacaatcctaacgtagaacaggatcacctggaagtgttaccgtcaatcgcctcaccta

SEQ ID NO: 194 - DNA, 209 bp, Artificial Sequence
  5′ RNA spacer + ZIKA-ph PEL + RNA XQ
cuuguggaaaggacgaaacaccugucaggccugcuagucagccacaguuuggggaaagcugugcagccuguaacccccccaggagaagcugggaaaccaagcuaaacatgacagaaatacgaacgaaactaacgcgcaaagatcgcaattaaacaatcctaacgtagaacaggatcacctggaagtgttaccgtcaatcgcctcaccta

SEQ ID NO: 195 - DNA, 359 bp, Artificial Sequence
  5′ RNA spacer + WNV-ph+ph PEL + RNA XQ
cuuguggaaaggacgaaacaccagucaggccagauuaaugcugccaccggaaguugaguagacggugcugccugcggcucaaccccaggaggacugggugaccaaagcugcgaggugauccacguaagcccucagaaccgucucggaaggaggaccccacgugcuuuagccucaaagcccagugucagaccacacuuuaaugugccacucugcggagagugcagucugcgauagugccccagguggacuggguaaacatgacagaaatacgaacgaaactaacgcgcaaagatcgcaattaaacaatcctaacgtagaacaggatcacctggaagtgttaccgtcaatcgcctcaccta

SEQ ID NO: 196 - DNA, 189 bp, Artificial Sequence
  5′ RNA spacer + WNV-p PEL + RNA XQ
cuuguggaaaggacgaaacaccagucaggccagauuaaugcugccaccggaaguugaguagacggugcugccugcggcucaacaaacatgacagaaatacgaacgaaactaacgcgcaaagatcgcaattaaacaatcctaacgtagaacaggatcacctggaagtgttaccgtcaatcgcctcaccta

SEQ ID NO: 197 - DNA, 440 bp, Artificial Sequence
  5′ RNA spacer + MVE_element PEL + RNA XQ
cuuguggaaaggacgaaacacccucgaggugagcaagggcgaggagcuguucaccgggguggugcccauccugguggacuugauagucaggccagccgguuaggcugccaccgaagguugguagacggugcugccugcgaccaaccccaggaggacuggguuaccaaagcugauucuccacgguuggaaagccucccagaaccgucucggaagaggagucccugccaacaauggagaugaagcccgugucagaucgcgaaagcgccacuucgccgaggagugcaaucugugaggccccaggaggacuggguaaacagucgacccgggcggccgcaaacatgacagaaatacgaacgaaactaacgcgcaaagatcgcaattaaacaatcctaacgtagaacaggatcacctggaagtgttaccgtcaatcgcctcaccta

SEQ ID NO: 198 - DNA, 267 bp, Artificial Sequence
  5′ RNA spacer + spacer_MVE-ph PEL + RNA XQ
cuuguggaaaggacgaaacacccucgaggugagcaagggcgaggagcuguucaccgggguggugcccauccugguggacuugauagucaggccagccgguuaggcugccaccgaagguugguagacggugcugccugcgaccaaccccaggaggacuggguaaacatgacagaaatacgaacgaaactaacgcgcaaagatcgcaattaaacaatcctaacgtagaacaggatcacctggaagtgttaccgtcaatcgcctcaccta

SEQ ID NO: 199 - DNA, 206 bp, Artificial Sequence
  5′ RNA spacer + MVE-ph PEL + RNA XQ
cuuguggaaaggacgaaacaccuagucaggccagccgguuaggcugccaccgaagguugguagacggugcugccugcgaccaaccccaggaggacuggguaaacatgacagaaatacgaacgaaactaacgcgcaaagatcgcaattaaacaatcctaacgtagaacaggatcacctggaagtgttaccgtcaatcgcctcaccta

SEQ ID NO: 200 - DNA, 190 bp, Artificial Sequence
  5′ RNA spacer + MVE-p PEL + RNA XQ
cuuguggaaaggacgaaacaccuagucaggccagccgguuaggcugccaccgaagguugguagacggugcugccugcgaccaacaaacatgacagaaatacgaacgaaactaacgcgcaaagatcgcaattaaacaatcctaacgtagaacaggatcacctggaagtgttaccgtcaatcgcctcaccta

SEQ ID NO: 201 - RNA, 173 nt, Artificial Sequence
  5′ RNA spacer + Dengue4-p PEL + RNA XQ
gaagacaacgaaacaccagucaggccacuugugccacgguuugagcaaaccgugcugccuguagcucaaacaugacagaaauacgaacgaaacuaacgcgcaaagaucgcaauuaaacaauccuaacguagaacaggaucaccuggaaguguuaccgucaaucgccucaccua

SEQ ID NO: 202 - RNA, 221 nt, Artificial Sequence
  5′ RNA spacer + OPMV PEL + RNA XQ
gaagacaacgaaacaccgaauugccuccaccaguaacuaaacccaaccacagccaagcauuaaguugcaagcguuggaguggcaggcuuaacguccgacaguacgacaacugcggaaacaugacagaaauacgaacgaaacuaacgcgcaaagaucgcaauuaaacaauccuaacguagaacaggaucaccuggaaguguuaccgucaaucgccucaccua

SEQ ID NO: 203 - RNA, 206 nt, Artificial Sequence
  5′ RNA spacer + PLRV PEL + RNA XQ
gaagacaacgaaacaccgccaccacaaaagaacacugaaggagcucacuaaaacuagccaagcauacacgaguugcaagcauuggaaguucaagccucguaaacaugacagaaauacgaacgaaacuaacgcgcaaagaucgcaauuaaacaauccuaacguagaacaggaucaccuggaaguguuaccgucaaucgccucaccua

SEQ ID NO: 204 - RNA, 212 nt, Artificial Sequence
  5′ RNA spacer + MODV PEL + RNA XQ
gaagacaacgaaacaccaugaaagaguguugagggcaaccagugggcuagccacauggguaugacgcacccacccucugcauucuuguaaauacuuuggccagucaaaacaugacagaaauacgaacgaaacuaacgcgcaaagaucgcaauuaaacaauccuaacguagaacaggaucaccuggaaguguuaccgucaaucgccucaccua

SEQ ID NO: 205 - RNA, 185 nt, Artificial Sequence
  TABV-1 PEL + RNA XQ
gaaaggcaagguacggauuagccguaggggcuugagaacccccccuccccacucauuuuauuuccucuaugaggaagguaaacaugacagaaauacgaacgaaacuaacgcgcaaagaucgcaauuaaacaauccuaacguagaacaggaucaccuggaaguguuaccgucaaucgccucaccua

SEQ ID NO: 206 - RNA, 187 nt, Artificial Sequence
  CXFV-1 PEL + RNA XQ
accccguaaggaaggacaaggcuguccuugaguacuaacgacacuccggccccaguucccagagccaggguuuuagcuccaaaacaugacagaaauacgaacgaaacuaacgcgcaaagaucgcaauuaaacaauccuaacguagaacaggaucaccuggaaguguuaccgucaaucgccucaccua

SEQ ID NO: 207 - RNA, 213 nt, Artificial Sequence
  5′ RNA spacer + MMLV PEL + RNA XQ
gaagacaacgaaacaccggaaaaaaugcgagugagggcaacucugggauuagcucaaugggugugacgacccuacccuuccgcauuuguaaauaauugagccagucaaaacaugacagaaauacgaacgaaacuaacgcgcaaagaucgcaauuaaacaauccuaacguagaacaggaucaccuggaaguguuaccgucaaucgccucaccua

SEQ ID NO: 208 - RNA, 219 nt, Artificial Sequence
  5′ RNA spacer + APOIV PEL + RNA XQ
gaagacaacgaaacaccauaagcgccuggagagcgaccuuggacuguggggguccaaagggcuuugcgacccuucucucuuugagcgcuuugggacaacuauuuggccagcauaaacaugacagaaauacgaacgaaacuaacgcgcaaagaucgcaauuaaacaauccuaacguagaacaggaucaccuggaaguguuaccgucaaucgccucaccua

SEQ ID NO: 209 - RNA, 195 nt, Artificial Sequence
  5′ RNA spacer + Wesselbron PEL + RNA XQ
gaagacaacgaaacaccagucagcccaucauuugaugccauggcuaagcugugaggccaugcuggcugggacagccgcgaccacccgcgaaacaugacagaaauacgaacgaaacuaacgcgcaaagaucgcaauuaaacaauccuaacguagaacaggaucaccuggaaguguuaccgucaaucgccucaccua

SEQ ID NO: 210 - RNA, 200 nt, Artificial Sequence
  5′ RNA spacer + Chaoyang PEL + RNA XQ
gaagacaacgaaacaccagucaggccuaaaugccaccggaugauaguagacggugcugccugcagcuaucacauauaaacuggcgccuuauaugaaacaugacagaaauacgaacgaaacuaacgcgcaaagaucgcaauuaaacaauccuaacguagaacaggaucaccuggaaguguuaccgucaaucgccucaccua

SEQ ID NO: 211 - RNA, 178 nt, Artificial Sequence
  5′ RNA spacer + WNV-2 PEL + RNA XQ
gaagacaacgaaacaccugucagaccacacuuuaaugugccacucugcggagagugcagucugcgauagugcaaacaugacagaaauacgaacgaaacuaacgcgcaaagaucgcaauuaaacaauccuaacguagaacaggaucaccuggaaguguuaccgucaaucgccucaccua

SEQ ID NO: 212 - RNA, 193 nt, Artificial Sequence
  5′ RNA spacer + WNV-4  PEL + RNA XQ
gaagacaacgaaacaccugucagaccacacuuuaaugugccacucugcggagagugcagucugcgauagugccccagguggacugggaaacaugacagaaauacgaacgaaacuaacgcgcaaagaucgcaauuaaacaauccuaacguagaacaggaucaccuggaaguguuaccgucaaucgccucaccua

SEQ ID NO: 213 - RNA, 185 nt, Artificial Sequence
  5′ RNA spacer + CFAV-2-ph PEL + RNA XQ
gaagacaacgaaacaccagcagggcacauuagugucgggcgugacgcacccgcuccccucaguccccugugcaacagggaaacaugacagaaauacgaacgaaacuaacgcgcaaagaucgcaauuaaacaauccuaacguagaacaggaucaccuggaaguguuaccgucaaucgccucaccua

SEQ ID NO: 214 - RNA, 198 nt, Artificial Sequence
  5′ RNA spacer + CXFV3-ph PEL + RNA XQ
gaagacaacgaaacaccccaucgcaagggaggauuuuccucggguacugaccauaccccgaccccaguccgauaggucauggaaugaccccaaaacaugacagaaauacgaacgaaacuaacgcgcaaagaucgcaauuaaacaauccuaacguagaacaggaucaccuggaaguguuaccgucaaucgccucaccua

SEQ ID NO: 215 - RNA, 167 nt, Artificial Sequence
  5′ RNA spacer + RCNMV-1 PEL + RNA XQ
gaagacaacgaaacaccgcguagccuccacccgaguugcaagaggggaacgcgcagucucgaaacaugacagaaauacgaacgaaacuaacgcgcaaagaucgcaauuaaacaauccuaacguagaacaggaucaccuggaaguguuaccgucaaucgccucaccua

SEQ ID NO: 216 - RNA, 167 nt, Artificial Sequence
  5′ RNA spacer + SCNMV PEL + RNA XQ
gaagacaacgaaacaccgcguaaccuccauccgaguugcaagagagggaaacgcagucucgaaacaugacagaaauacgaacgaaacuaacgcgcaaagaucgcaauuaaacaauccuaacguagaacaggaucaccuggaaguguuaccgucaaucgccucaccua

SEQ ID NO: 217 - RNA, 169 nt, Artificial Sequence
  5′ RNA spacer + CRSV PEL + RNA XQ
gaagacaacgaaacaccccguagccgccaacaaaguugcaagagcgggcguugcuagccuuugaaacaugacagaaauacgaacgaaacuaacgcgcaaagaucgcaauuaaacaauccuaacguagaacaggaucaccuggaaguguuaccgucaaucgccucaccua

SEQ ID NO: 218 - RNA, 253 nt, Artificial Sequence
  5′ RNA spacer + RCNMV-2 PEL + RNA XQ
gaagacaacgaaacaccgcguagccuccacccgaguugcaagaggggaacgcgcagucucgccgacccuguuggcaaacaguaaaauugcaaaaaauagagugcuaggaguaguucccguacccgcgggagcaagacccuacuacagaaacaugacagaaauacgaacgaaacuaacgcgcaaagaucgcaauuaaacaauccuaacguagaacaggaucaccuggaaguguuaccgucaaucgccucaccua

SEQ ID NO: 219 - RNA, 161 nt, Artificial Sequence
  5′ RNA spacer + Rbsw-1 PEL + RNA XQ
gaagacaacgaaacaccacgcgguucuaucuaguuacgcguuaaaccaacuagaaaaacaugacagaaauacgaacgaaacuaacgcgcaaagaucgcaauuaaacaauccuaacguagaacaggaucaccuggaaguguuaccgucaaucgccucaccua

SEQ ID NO: 220 - RNA, 160 nt, Artificial Sequence
  5′ RNA spacer + Rbsw-2 PEL + RNA XQ
gaagacaacgaaacacccgcgguucuaucuaguuacgcguuaaaccaacuagaaaaacaugacagaaauacgaacgaaacuaacgcgcaaagaucgcaauuaaacaauccuaacguagaacaggaucaccuggaaguguuaccgucaaucgccucaccua

SEQ ID NO: 221 - RNA, 168 nt, Artificial Sequence
  5′ RNA spacer + Rbsw-3 PEL + RNA XQ
gaagacaacgaaacaccacgcgguucuaucuaguuacgcguuaaaccaacuagaaggcgguuaaacaugacagaaauacgaacgaaacuaacgcgcaaagaucgcaauuaaacaauccuaacguagaacaggaucaccuggaaguguuaccgucaaucgccucaccua